(19) United States
(12) Patent Application Publication
(10) Pub. No.: US 2026/0356849 A1
(43) Pub. Date: Nov. 12, 2026

(54) COMPOUNDS AND METHODS FOR TREATING CANCERS

(71) Applicant: CEDARS-SINAI MEDICAL CENTER, Los Angeles, CA (US)

(72) Inventors: Shelly LU, Los Angeles, CA (US); Ramachandran MURALI, Beverly Hills, CA (US); James TURKSON, Pacific Palisades, CA (US); Francisco LOPEZ TAPIA, Santa Clarita, CA (US)

(73) Assignee: CEDARS-SINAI MEDICAL CENTER, Los Angeles, CA (US)

(21) Appl. No.: 19/123,196

(22) PCT Filed: Nov. 3, 2023
(86) PCT No.: PCT/US2023/078657

§ 371 Date: Apr. 22, 2025

Related U.S. Application Data
(60) Provisional application No. 63/422,672, filed on Nov. 4, 2022.

(51) Int. Cl. C07D 495/22; A61K 31/519; A61K 31/5377; A61P 35/00

(52) U.S. Cl. CPC C07D 495/22; A61K 31/519; A61K 31/5377; A61P 35/00

(57) ABSTRACT

The present invention describes compounds that inhbits GIT1. The invention further describes compositions containing these GIT1 inhibitors, as well as methods and kits using these GIT1 inhibitors to treat various medical conditions. Medical conditions treatable with various embodiments of the invention include but are not limited to cancers and tumors.

### CROSS-REFERENCE TO RELATED APPLICATIONS

This application claims the benefit of priority under 35 U.S.C. § 119(e) to U.S. Provisional Patent Application No. 63/422,672 filed Nov. 4, 2022, the contents of which is incorporated herein by reference in its entirety.

### REFERENCE TO SEQUENCE LISTING

This application contains a sequence listing submitted as an electronic xml file named, “065472-000924WOPT” created on Oct. 30, 2023 and having a size in bytes of 3,483 bytes. The information contained in this electronic file is hereby incorporated by reference in its entirety.

### FIELD OF INVENTION

This invention relates to compounds, compositions, and methods for treating a medical condition. The condition includes but is not limited to a cancer or tumor.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Liver cancer is the fourth most common cause of cancer related death with a 5-year survival of 18%. Colorectal cancer (CRC) is the second and third most common cancer diagnosed in women and men, respectively. Approximately 35% of CRC patients present with metastatic colorectal cancer (mCRC) at time of diagnosis and as many as 50% of patients with initially localized CRC develop mCRC later. Majority of hepatocellular carcinoma (HCC, the most common form of primary liver cancer) patients present late and only 30% are candidates for surgical resection but, unfortunately, still face a recurrence rate of about 50% at 3 years. Much progress has been made in treating mCRC and advanced HCC, but the overall prognosis remains poor. Although traditional chemo/immunotherapies have improved survival, they often have significant adverse side effects, and many cancers develop resistance. Therefore, there is a need for targeted treatment strategies using small molecule inhibitors. The embodiments of the present invention address that need.

### SUMMARY OF THE INVENTION

In various embodiments, the present invention provides a compound of Formula (A):

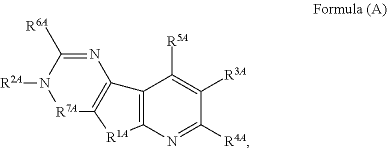

wherein:
R^{1A} is S, O, CH₂, C(CH₃)₂, or NR^{8A}, where R^{8A} is H or optionally substituted alkyl; R^{2A} is H, optionally substituted alkyl, optionally substituted carbonyl, optionally substituted aryl, optionally substituted heteroaryl, optionally substituted cyclyl, or optionally substituted heterocyclyl; R^{3A} is H, halo, optionally substituted alkyl, or OR^{9A}, where R^{9A} is H or optionally substituted alkyl; R^{4A} is H, halo, optionally substituted alkyl, or OR^{10A}, where R^{10A} is H or optionally substituted alkyl; R^{5A} is H, halo, or optionally substituted alkyl; R^{6A} is H, halo, or optionally substituted alkyl; and R^{7A} is C═O or CH₂; or R^{3A} and R^{4A} can be linked together to form a ring, wherein the ring is optionally substituted; or R^{3A} and R^{5A} can be linked together to form a ring, wherein the ring is optionally substituted. In some embodiments, the compound of Formula (A) is not

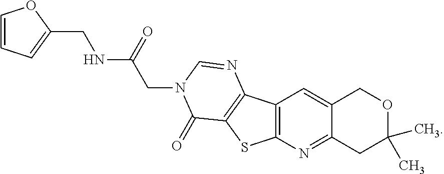 [SMILES: CC1(C)Cc2nc3sc4c(=O)n(CC(=O)NCc5ccco5)cnc4c3cc2CO1]

In some embodiments, the compound of Formula (A) is

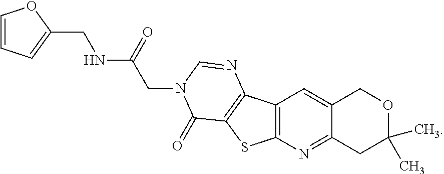 [SMILES: CC1(C)Cc2nc3sc4c(=O)n(CC(=O)NCc5ccco5)cnc4c3cc2CO1]

In some embodiments, the compound of Formula (A) is:

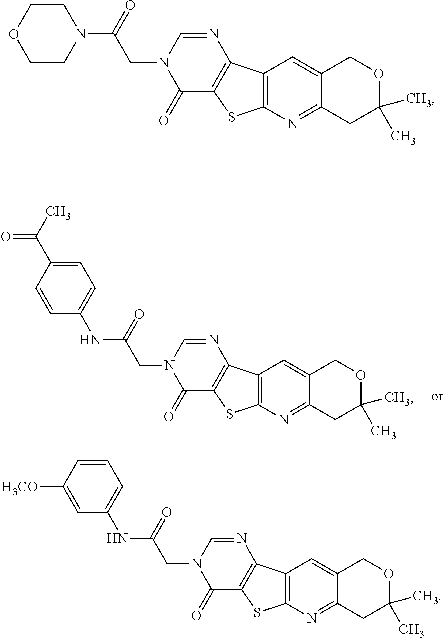 [SMILES: CC1(C)Cc2nc3sc4c(=O)n(CC(=O)N5CCOCC5)cnc4c3cc2CO1 ; CC(=O)c1ccc(NC(=O)Cn2cnc3c(sc4nc5c(cc43)COC(C)(C)C5)c2=O)cc1 ; "or" ; COc1cccc(NC(=O)Cn2cnc3c(sc4nc5c(cc43)COC(C)(C)C5)c2=O)c1]

In various embodiments, the present invention provides a pharmaceutical composition comprising an inhibitor of GIT1's normal interactome. In some embodiments, the GIT1 inhibitor is a compound of the present invention.

In various embodiments, the present invention provides a method of treating, preventing, reducing the likelihood of having, reducing the severity of and/or slowing the progression of a condition in a subject comprising: administering a therapeutically effective amount of an inhibitor of GIT1's normal interactome to the subject, thereby treating, preventing, reducing the likelihood of having, reducing the severity of and/or slowing the progression of the condition in the subject, wherein the condition is cancer or a condition associated with an increased expression of GIT1 and MAT2B. In some embodiments, the GIT1 inhibitor is a compound of the present invention.

In various embodiments, the present invention provides kit for treating, preventing, or reducing the likelihood of having, reducing the severity of and/or slowing the progression of a condition in a subject, comprising: an inhibitor of GIT1's normal interactome; and instructions for using the GIT1 inhibitor to treat, prevent, reduce the likelihood of having, reduce the severity of and/or slow the progression of the condition in the subject. In some embodiments, the GIT1 inhibitor is a compound of the present invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with various embodiments of the invention, the structures of non-limiting examples of the inventors' compounds that inhibit GIT1-MAT2B interaction.

FIG. 2 depicts, in accordance with various embodiments of the invention, the inventors' novel strategy of targeting the ANK domain of GIT1 with a molecule.

FIG. 3 depicts, in accordance with various embodiments of the invention, the interaction between FLAG-tagged GIT1 constructs of variable lengths and endogenous MAT2B show interaction lies between 250-420 amino acids of GIT1, corresponding to the SHD domain.

FIG. 4 depicts, in accordance with various embodiments of the invention, the growth inhibitory effect of compound C3.

FIG. 5 depicts, in accordance with various embodiments of the invention, the growth inhibitory effect of compound C3.

FIG. 6 depicts, in accordance with various embodiments of the invention, that compound C3 induces cell cycle arrest in liver cancer cells at G2/M, but not non-malignant cells.

FIG. 7 depicts, in accordance with various embodiments of the invention, that compound C3 induces cell cycle arrest in colon cancer cells at G2/M, but not non-malignant cells.

FIG. 8 depicts, in accordance with various embodiments of the invention, results of an annexin V-FITC/propidium iodide binding assay performed in human and mouse hepatocytes, AML12, HEK293, HepG2, and RKO cells after treatment with 5 μM of a GIT1 inhibitor or DMSO for 24-48 hours (results are shown as mean±SE from at least 3 experiments. *p<0.05 vs. DMSO).

FIG. 9 depicts, in accordance with various embodiments of the invention, compound C3 inhibits both clonogenic potential and migration in cancer cells.

FIG. 10 depicts, in accordance with various embodiments of the invention, compound C3 inhibits both clonogenic potential and migration in cancer cells.

FIG. 11 depicts, in accordance with various embodiments of the invention, compound C3 disrupts MAT2B-GIT1 interaction.

FIG. 12 depicts, in accordance with various embodiments of the invention, compound C3 disrupts interaction of MEK1/2 with GIT1, Raf, and ERK.

FIG. 13 depicts, in accordance with various embodiments of the invention, compound C3 inhibits ERK activity and cyclin D1 expression in cancer cells but not in non-malignant cells.

FIG. 14 depicts, in accordance with various embodiments of the invention, compound C3 inhibits MEK activity in cancer cells but not in non-malignant cells.

FIG. 15 depicts, in accordance with various embodiments of the invention, results of treating HepG2 and RKO cells with DMSO or compound C3 for 24 hours and blotting for phospho-γH2AX (S139) and cyclin B1.

FIG. 16A-FIG. 16C depicts, in accordance with various embodiments of the invention, compound C3 increases CyclinB1 mRNA level and inhibits its protein degradation.

FIG. 17A-FIG. 17C depicts, in accordance with various embodiments of the invention, higher CyclinB1 enhances CDK1/CyclinB1 binding and raises CDK1 activity to promote CDK1 nuclear entry.

FIG. 18A-FIG. 18C depicts, in accordance with various embodiments of the invention, CyclinB1 directly interacts with GIT1 and compound C3 promotes their interaction.

FIG. 19A-FIG. 19E shows that compound C3 inhibits colon cancer growth in a syngeneic (C57/BL6) subcutaneous mouse model.

FIG. 20A-FIG. 20C shows that compound C3 inhibits human colon cancer cells growth in liver of nude mice.

FIG. 21A-FIG. 21E shows that compound C3 inhibits colon cancer liver metastasis in immune competent mice model.

FIG. 22A-FIG. 22B shows that treatment of compound C3 reduces M2 macrophages in the tumor microenvironment.

FIG. 23 show that compound C3 binds to GIT1-GFP and not GIT2-GFP using MST.

FIG. 24 depicts, in accordance with various embodiments of the invention, the predicted interactions of GIT1-Cyclin BI using PRISM software. GIT1's hydrophobic aliphatic residues at 730, 731, and 736 are visualized in the zoomed image on the right panel.

FIG. 25 shows that GIT1 mutation lowers interaction with cyclin B1.

FIG. 26 shows that compound C3 inhibits growth of CRC organoid.

FIG. 27 depicts infographics about liver and colon cancers.

FIG. 28 depicts, in accordance with various embodiments of the invention, G protein-coupled receptor kinase-interacting protein 1 (GIT1).

FIG. 29 depicts, in accordance with various embodiments of the invention, methionine Adenosyltransferase 2B (MAT2B).

FIG. 30 depicts, in accordance with various embodiments of the invention, GIT1-MAT2B as a scaffold complex that interacts with MEK and ERK to regulate MAPK activity in liver and colon cancers.

FIG. 31 depicts, in accordance with various embodiments of the invention, that MAT2B and GIT1 interact and are overexpressed in human HCC.

FIG. 32 depicts, in accordance with various embodiments of the invention, the role of GIT1-MAT2B in Ras signaling.

FIG. 33 depicts, in accordance with various embodiments of the invention, compound C3 induces a slight increase in apoptosis in cancer cells.

FIG. 34 depicts, in accordance with various embodiments of the invention, compound C3 increases CyclinB1 content and promotes CyclinB1 phosphorylation that is associated with nuclear accumulation.

FIG. 35 depicts, in accordance with various embodiments of the invention, compound C3-mediated stabilization of CyclinB1 is GIT1 dependent.

FIG. 36 depicts, in accordance with various embodiments of the invention, no toxicity of compound C3 in C57/BL6 mice.

FIG. 37 depicts, in accordance with various embodiments of the invention, a summary of the scheme of inhibition of GIT1.

FIG. 38A-FIG. 38D depicts, in accordance with various embodiments of the invention, compound C3 enhances GIT1-cyclin BI interaction and cyclin B1 expression, lowers growth via GIT1, In vitro translated GIT1, rMAT2B and cyclin B1 (both 0.5uG) were used to evaluate direct binding (FIG. 38A). HepG2 cells were treated with compound C3 at 10 μM for 24 h and subjected to co-IP with anti-GIT1 or anti-cyclin B1 and blotted for cyclin B1 and GIT1, ratios of cyclin B1 bound to GIT1 vs. DMSO control are shown, *p<0.05 vs. DMSO from n=3 (FIG. 38B). Compound C3-mediated increase in cyclin BI level (FIG. 38C) and lower EdU requires GIT1 (FIG. 38D).

FIG. 39A-FIG. 39D depicts, in accordance with various embodiments of the invention, compound C3 inhibits human CRC growth in liver. RKO cells (2×10⁶) stably expressing luciferase were injected into the liver of nude mice. Twenty days later (day 0) DMSO and compound C3 were injected intraperitoneally (ip) in FIG. 39A pilot experiment using 10 mg/kd/d×5 days, rest 2 days and repeat. FIG. 39B 25 mg/kg daily for 5 days and imaged on day 7 using IVIS Spectrum photon-counting device optical imaging system (Xenogen, Alameda, CA.). FIG. 39C graph summarizes luciferase intensity from FIG. 39B. FIG. 39D gross pictures of the livers at day 7 from FIG. 39B, point to tumors *p<0.05.

FIG. 40A-FIG. 40E depicts, in accordance with various embodiments of the invention, compound C3 inhibits CRC liver metastasis in immune competent mice. MC38 cells (2.5×10⁵) stably expressing luciferase where injected intrasplenically followed by splenectomy. 7 days later compound C3 (20 mg/kg on day 1 sinc MC38 grows rapidly, then 15 mg/kg daily×3 days, rest for 2 days, then 15 mg/kg×2 days) or DMSO were given ip. Small animal imaging (FIG. 40A). H&E of tumors (FIG. 40B). Luciferase intensity at various time points (FIG. 40C). Gross pictures of the livers at day 9 (FIG. 40D), ALT and AST levels after above, or compound C3 (15 mg/kg/d for 21 days), *p<0.05 vs. DMSO (FIG. 40E).

FIG. 41 depicts, in accordance with various embodiments of the invention, medicial chemistry strategy for C3 analog compound affinity and selectivity.

FIG. 42 depicts, in accordance with various embodiments of the invention, effects of Compound C33 on MTT in RKO and HepG2 cells.

FIG. 43 depicts, in accordance with various embodiments of the invention, general synthetic preparation of Compound C3 and some analogs.

FIG. 44 depicts, in accordance with various embodiments of the invention, modeling of compound C3 bound to its predicted GIT1 active site.

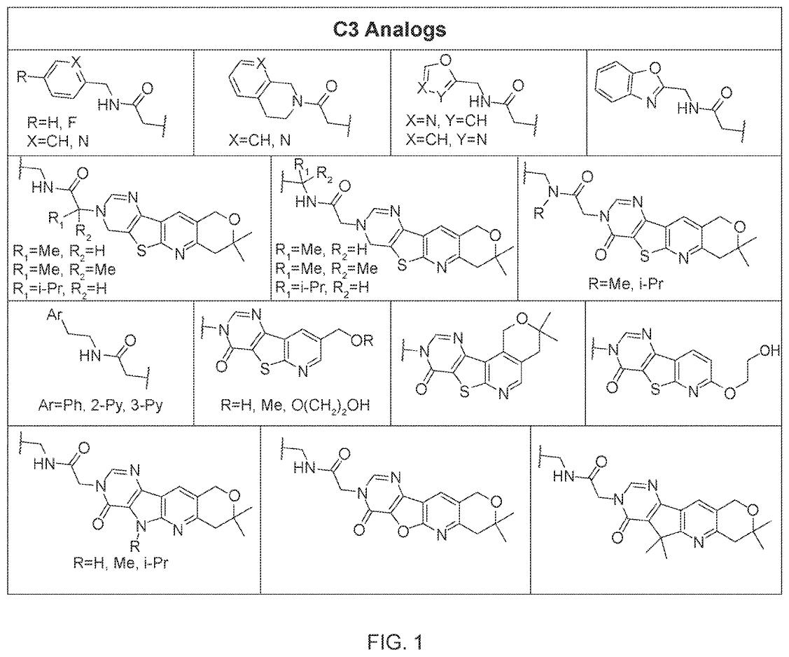

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., Revised, J. Wiley & Sons (New York, NY 2006); March, Advanced Organic Chemistry Reactions, Mechanisms and Structure 7th ed., J. Wiley & Sons (New York, NY 2013); and Sambrook and Russel, Molecular Cloning: A Laboratory Manual 4th ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of cancer or tumor, delay or slowing of cancer or tumor, and amelioration or palliation of symptoms associated with cancer or tumor.

“Conditions” and “disease conditions,” as used herein may include, but are in no way limited to any form of malignant neoplastic cell proliferative disorders or diseases. Examples of such disorders include but are not limited to cancer and tumor.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant cancers, as well as dormant tumors or micrometastasis. Cancers which migrate from their original location and seed vital organs can eventually lead to the death of the subject through the functional deterioration of the affected organs. As used herein, the term “invasive” refers to the ability to infiltrate and destroy surrounding tissue.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

The term “sample” or “biological sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy.

As used herein, a “subject” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., cancer or tumor) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

As used herein the term “electron donating group” is well-known in the art and generally refers to a functional group or atom that pushes electron density away from itself, towards other portions of the molecule, e.g., through resonance and/or inductive effects. Non-limiting examples of electron-donating groups include OR^{c}, NR^{c}R^{d}, alkyl groups, wherein R^{c} and R^{d} are each independently H, optionally substituted alkyl, optionally substituted heteroalkyl, optionally substituted aryl, optionally substituted heteroaryl, optionally substituted cyclyl, or optionally substituted heterocyclyl.

As used herein the term “electron withdrawing group” is well-known in the art and generally refers to a functional group or atom that pulls electron density towards itself, away from other portions of the molecule, e.g., through resonance and/or inductive effects. Non-limiting examples of electron withdrawing groups include NO₂, F, Cl, Br, I, CF₃, CN, CO₂R^{a}, C(═O) NR^{a}R^{b}, C(═O)R^{a}, SO₂R^{a}, SO₂OR^{a}, SO₂NR^{a}R^{b}, PO₃R^{a}R^{b}, or NO, wherein R^{a} and R^{b} are each independently H, optionally substituted alkyl, optionally substituted heteroalkyl, optionally substituted aryl, optionally substituted heteroaryl, optionally substituted cyclyl, or optionally substituted heterocyclyl.

As used herein, the term “aliphatic” means a moiety characterized by a straight or branched chain arrangement of constituent carbon atoms and can be saturated or partially unsaturated with one or more (e.g., one, two, three, four, five or more) double or triple bonds.

As used herein, the term “alicyclic” means a moiety comprising a nonaromatic ring structure. Alicyclic moieties can be saturated or partially unsaturated with one or more double or triple bonds. Alicyclic moieties can also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C3-C8 rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀) aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S. The term “alkyl” includes heteroalkyl.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Non-limiting examples of substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 2 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃) CH₂CH—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Non-limiting examples of Rₐ and R_{b} are each independently hydrogen, alkyl, substituted alkyl, alkenyl, or substituted alkenyl. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine (F), chlorine (Cl), bromine (Br) and iodine (I). The term “halogen radioisotope” or “halo radioisotope” refers to a radionuclide of an atom selected from fluorine (F), chlorine (Cl), bromine (Br) and iodine (I).

In some embodiments, “iodo” refers to the iodine atom (I) when it is used in the context of a halo functional group or halogen functional group or as a halo substituent or halogen substituent.

In some embodiments, “bromo” refers to the bromine atom (Br) when it is used in the context of a halo functional group or halogen functional group or as a halo substituent or halogen substituent.

In some embodiments, “chloro” refers to the chlorine atom (Cl) when it is used in the context of a halo functional group or halogen functional group or as a halo substituent or halogen substituent.

In some embodiments, “fluoro” refers to the fluorine atom (F) when it is used in the context of a halo functional group or halogen functional group or as a halo substituent or halogen substituent.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. For example, C₆-C₁₂ aryl includes aryls that have 6 to 12 carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-l-yl, decahydronaphthyl, oxocyclohexyl, dioxocyclohexyl, thiocyclohexyl, 2-oxobicyclo[2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heterocyclyl” refers to a nonaromatic 5-8 membered monocyclic, 4-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp2 hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

Substituents may be protected as necessary and any of the protecting groups commonly used in the art may be employed. Non-limiting examples of protecting groups may be found, for example, in Greene et al., Protective Groups in Organic Synthesis, 3rd Ed. (New York: Wiley, 1999).

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of—O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl) z, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C2-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O) OCH₃, —C(═O) OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH (alkyl) or —N(alkyl) (alkyl), respectively, such as —NHCH₃, —N(CH₃)₂, and the like.

The term “mono- or di-alkylaminoalkoxy” means —O-(alkyl)-NH (alkyl) or —O-(alkyl)-N(alkyl)(alkyl), respectively, such as —OCH₂NHCH₃, —OCH₂CH₂N(CH₃)₂, and the like.

The term “arylamino” means —NH (aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH (alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino”—NH-(alkyl)-(cycloalkyl), such as —NHCH₂-cyclohexyl, and the like.

The terms “cyclic” and “ring” refer to alicyclic or aromatic groups that may or may not be substituted and/or that may or may not be heteroatom containing, and that may be monocyclic, bicyclic, or polycyclic. In some embodiments, the ring is optionally substituted.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

The term “derivative” as used herein refers to a chemical substance related structurally to another, i.e., an “original” substance, which can be referred to as a “parent” compound. A “derivative” can be made from the structurally-related parent compound in one or more steps. In some embodiments, the general physical and chemical properties of a derivative can be similar to or different from the parent compound.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

In various embodiments, compounds of the present invention as disclosed herein may be synthesized using any synthetic method available to one of skill in the art. Non-limiting examples of synthetic methods used to prepare various embodiments of compounds of the present invention are disclosed in the Examples section herein.

A “pharmaceutically acceptable salt”, as used herein, is intended to encompass any compound described herein that is utilized in the form of a salt thereof, especially where the salt confers on the compound improved pharmacokinetic properties as compared to the free form of compound or a different salt form of the compound. The pharmaceutically acceptable salt form can also initially confer desirable pharmacokinetic properties on the compound that it did not previously possess, and may even positively affect the pharmacodynamics of the compound with respect to its therapeutic activity in the body. An example of a pharmacokinetic property that can be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Pharmaceutically acceptable salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), the content of which is herein incorporated by reference in its entirety. Exemplary salts also include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. Suitable acids which are capable of forming salts with the compounds of the disclosure include inorganic acids such as hydrochloric acid, hydrobromic acid, perchloric acid, nitric acid, thiocyanic acid, sulfuric acid, phosphoric acid, and the like; and organic acids such as 1,2-ethanedisulfonic acid, 2-hydroxyethanesulfonic acid, 2-naphthalenesulfonic acid, 3-phenylpropionic acid, 4-methylbicyclo[2.2.2]oct-2-ene-1-carboxylic acid, 4,4′-methylenebis(3-hydroxy-2-ene-1-carboxylic acid), acetic acid, anthranilic acid, benzenesulfonic acid, benzoic acid, camphorsulfonic acid, cinnamic acid, citric acid, cyclopentanepropionic acid, ethanesulfonic acid, formic acid, fumaric acid, glucoheptonic acid, gluconic acid, glutamic acid, glycolic acid, heptanoic acid, hydroxynaphthoic acid, lactic acid, lauryl sulfuric acid, maleic acid, malic acid, malonic acid, mandelic acid, methanesulfonic acid, muconic acid, naphthalene sulfonic acid, o-(4-hydroxybenzoyl)benzoic acid, oxalic acid, p-chlorobenzenesulfonic acid, propionic acid, p-toluenesulfonic acid, pyruvic acid, salicylic acid, stearic acid, succinic acid, sulfanilic acid, tartaric acid, tertiary butylacetic acid, trifluoroacetic acid, trimethylacetic acid, and the like. Suitable bases capable of forming salts with the compounds of the disclosure include inorganic bases such as sodium hydroxide, ammonium hydroxide, sodium carbonate, calcium hydroxide, potassium hydroxide and the like; and organic bases such as mono-, di- and tri-alkyl and aryl amines (e.g., triethylamine, diisopropyl amine, methyl amine, dimethyl amine, N-methylglucamine, pyridine, picoline, dicyclohexylamine, N,N′-dibezylethylenediamine, and the like), and optionally substituted ethanol-amines (e.g., ethanolamine, diethanolamine, trierhanolamine and the like).

The term “protected derivatives” means derivatives of compounds described herein in which a reactive site or sites are blocked with protecting groups. Protected derivatives are useful in the preparation of compounds or in themselves can be active. A comprehensive list of suitable protecting groups can be found in T. W. Greene, Protecting Groups in Organic Synthesis, 3rd edition, John Wiley & Sons, Inc. 1999.

“Isomers” mean any compound having identical molecular formulae but differing in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality. A mixture of the two enantiomeric forms is termed a “racemic mixture”. A compound that has more than one chiral center has 2n−1 enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate planepolarized light in different directions). Enantiomers generally have identical physical properties, such as melting points and boiling points, and also have identical spectroscopic properties. Enantiomers can differ from each other with respect to their interaction with plane-polarized light and with respect to biological activity.

The designations “R” and “S” are used to denote the absolute configuration of the molecule about its chiral center(s). The designations may appear as a prefix or as a suffix, they may or may not be separated from the isomer by a hyphen; they may or may not be hyphenated; and they may or may not be surrounded by parentheses.

The designations or prefixes “(+)” and “(−)” are employed to designate the sign of rotation of plane-polarized light by the compound, with (−) meaning that the compound is levorotatory (rotates to the left). A compound prefixed with (+) is dextrorotatory (rotates to the right).

The term “racemic mixture,” “racemic compound” or “racemate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “resolving” or “resolution” when used in reference to a racemic mixture refers to the separation of a racemate into its two enantiomorphic forms (i.e., (+) and (−); 65 (R) and(S) forms). The terms can also refer to enantioselective conversion of one isomer of a racemate to a product.

“Optional” or “optionally” means that the subsequently described circumstance may or may not occur, so that the description includes instances where the circumstance occurs and instances where it does not.

In some embodiments, the numbers expressing quantities of reagents, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Correlation exists between over expression of ARF GTPase protein 1 (GIT1) and cancer progression in humans, including hepatocellular carcinoma (HCC) and colorectal cancer (CRC). GIT1 is able to form a complex with methionine adenosyltransferase 2B (MAT2B), which activates RAS-RAF-MEK-ERK signaling in HCC and CRC to enhance tumor growth and invasion. These observations indicate that inhibition of GIT1 is a potential treatment strategy for many cancers.

This invention demonstrates that inhibitors of GIT1, particularly those that target the GIT1 akyrin domain, lead to impaired RAF-MEK-ERK signaling, apoptosis, and cell cycle arrest, and provides compounds, compositions, and methods for treating various conditions including but not limited to cancers and tumors.

One problem addressed by the invention is related to spread of cancers (for example, colorectal cancer) by metastasis. compounds, compositions, methods, and kits that inhibit cancer spread by metastasis.

### GIT1 Inhibitor Compounds

In various embodiments, the present invention provides a compound that inhibits interaction between GIT1 and MAT2B.

In embodiments of the various aspects disclosed herein, the inhibitor compound is of Formula (I):

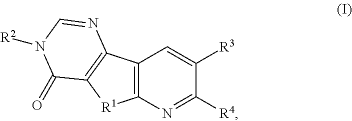

wherein:
R₁ is an amine, sulfur, oxygen, C(CH₃)₂, or methylene, each of which can be optionally substituted;
R₂ is a hydrogen, carbonyl, aromatic moiety, alkyl, acyl, cyclyl or heterocyclyl, each of which can be optionally substituted;
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

In some embodiments, the compound is not compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide). In some embodiments, the compound is compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

In some other embodiments of the various aspects disclosed herein, the compound is of Formula (II):

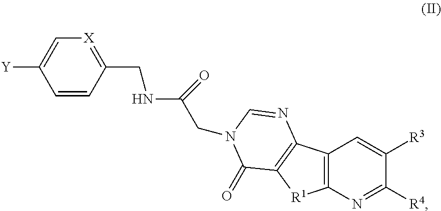

wherein:
X is a nitrogen or CH, which can be optionally substituted;
Y is a hydrogen, halide, nitrile, alkyl, acyl, or hydroxyl, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

In some other embodiments of the various aspects disclosed herein, the compound is of Formula (III):

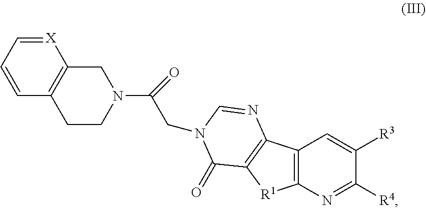

wherein:
X is methine or nitrogen;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

In some other embodiments of the various aspects disclosed herein, the compound is of Formula (IV):

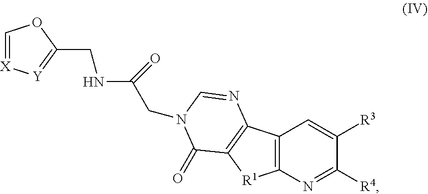

X is methine or nitrogen;
Y is methine or nitrogen;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

In some other embodiments of the various aspects disclosed herein, the compound is of Formula (V):

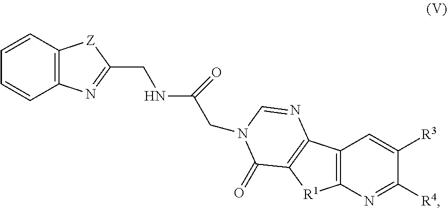

wherein:
Z is an oxygen or sulfur, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen of nitrogen atom.

In some other embodiments of the various aspects disclosed herein, the compound is of Formula (VI):

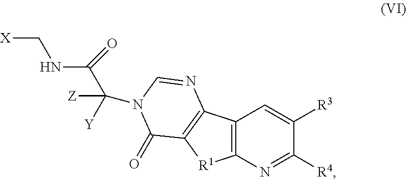

wherein:
X is an aromatic moiety, alkyl, acyl, heteroaryl, cyclyl or heterocyclyl, each of which can be optionally substituted;
Y and/or Z is a hydrogen, aromatic moiety, alkyl, acyl, cyclyl or heterocyclyl, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

In some other embodiments of the various aspects disclosed herein, the compound is of Formula (VII):

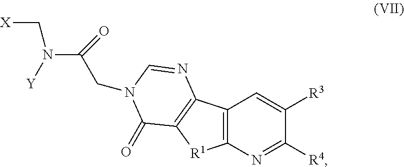

wherein:
X is an aromatic moiety, alkyl, acyl, cyclyl or heterocyclyl, each of which can be optionally substituted;
Y is an aromatic moiety, alkyl, acyl, cyclyl or heterocyclyl, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

In some other embodiments of the various aspects disclosed herein, the compound is of Formula (VIII):

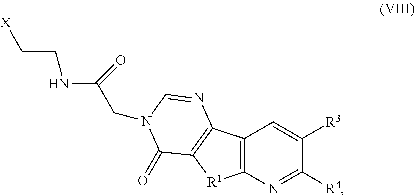

wherein:
X is an aromatic moiety, alkyl, acyl, cyclyl or heterocyclyl, aryl, or heteroaryl, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

In some other embodiments of the various aspects disclosed herein, the compound is of Formula (IX):

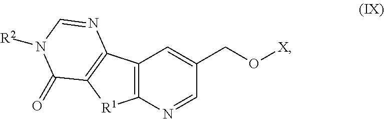

X is a hydrogen, lower alkyl, O(CH₂)₂OH, or alcohol, each of which can be optionally substituted;
R₁ is an amine, sulfur, oxygen, C(CH₃)₂, or methylene, each of which can be optionally substituted; and
R₂ is a hydrogen, carbonyl, aromatic moiety, alkyl, acyl, cyclyl or heterocyclyl, each of which can be optionally substituted.

In some other embodiments of the various aspects disclosed herein, the compound is of Formula (X):

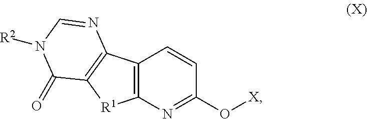

wherein:
X is a hydrogen, lower alkyl, O(CH₂)₂OH, or alcohol, each of which can be optionally substituted;
R₁ is an amine, sulfur, oxygen, C(CH₃)₂, or methylene, each of which can be optionally substituted; and
R₂ is a hydrogen, carbonyl, aromatic moiety, alkyl, acyl, cyclyl or heterocyclyl, each of which can be optionally substituted.

In some other embodiments of the various aspects disclosed herein, the compound is of Formula (XI):

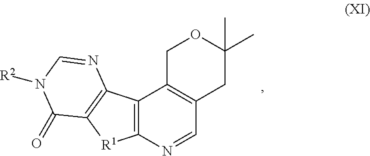

wherein:
R₁ is an amine, sulfur, oxygen, C(CH₃)₂, or methylene, each of which can be optionally substituted; and
R₂ is a hydrogen, carbonyl, aromatic moiety, alkyl, acyl, cyclyl or heterocyclyl, each of which can be optionally substituted.

As discussed herein, the GIT1 inhibitor can be a pharmaceutically acceptable salt, solvent, hydrate, polymorph, co-crystal, tautomer, stereoisomer, or isotopically labeled derivative of one or more of the above compounds.

In various embodiments, the IC50 of the GIT1 inhibitor is about 20 μM. In certain embodiments, the IC50 of the GIT1 inhibitor is less than 30 μM, less than 25 μM, less than 20 μM, less than 15 μM, less than 10 μM, less than 5 μM, or some combination thereof.

### GIT1 Inhibitor Compounds

In various embodiments, the present invention provides a compound that inhibits interaction between GIT1 and MAT2B.

In various embodiments of the present invention, the inhibitor compound is of Formula (A), Formula (A-1), Formula (A-2), Formula (A-3), or Formula (A-4).

In some embodiments, the compound is not compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

In some embodiments, the compound is compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

### Pharmaceutical Compositions

In various embodiments, the present invention provides a composition that consists of, consists essentially of, or comprises an inhibitor of GIT1 expression and/or activity. In accordance with the present invention, the composition can be used for treating, preventing, reducing the likelihood of having, reducing the severity of and/or slowing the progression of a condition in a subject.

In various embodiments, the inhibitor is a compound of Formula (I), Formula (II), Formula (III), Formula (IV), Formula (V), Formula (VI), Formula (VII), Formula (VIII), Formula (IX), Formula (X), Formula (XI), or a combination thereof. In some embodiments, the compound is not compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

In some embodiments, the compound is compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

In various embodiments, the inhibitor is a compound of Formula A, Formula A-1, Formula A-2, Formula A-3, Formula A-4, or a combination thereof. In some embodiments, the compound is not compound C3 (2-(8,8-Dimethyl-4-oxo-7,10-dihydro-8H-pyrano[3″,4″: 5′,6′]pyrido[3,2′: 4,5]thieno[3,2-d]pyrimidin-3 (4H)-yl)-N-(2-furylmethyl) acetamide). In some embodiments, the compound is compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

In some embodiments, the inhibitor further comprises a pharmaceutically acceptable carrier or excipient.

In various embodiments, the GIT1 inhibitor inhibits contacting between GIT1 and its normal interacting proteins including MAT2B. In some embodiments, the inhibitor of GIT1 inhibits contacting between GIT1 and MEK1, MEK2, and recruitment of cRAF, BRAF, and ERK to MEK1/2. In various embodiments, the inhibitor of GIT1 promotes contacting between GIT1 and cyclin B1. In various embodiments, the inhibitor of GIT1 promotes contacting between cyclin B1 and CDK1.

In various embodiments, the inhibitor may be administered 1-3 times per day, 1-7 times per week, 1-9 times per month, or more, so as to administer an effective amount of the inhibitor to the subject, where the effective amount is any one or more of the doses described herein. In some embodiments, the inhibitor is administered at about 0.1-0.5, 0.5-5, 5-10, 10-20, 20-50, 50-100, 100-200, 200-300, 300-400, 400-500, 500-600, 600-700, 700-800, 800-900, or 900-1000 μM, or a combination thereof. In various embodiments, the inhibitor of GIT1 is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the inhibitor is administered once, twice, three, or more times.

In various embodiments, the pharmaceutical compositions according to the invention may be formulated for delivery via any route of administration. “Route of administration” may refer to any administration pathway known in the art, including but not limited to intratumoral, aerosol, nasal, oral, transmucosal, transdermal or parenteral.

“Transdermal” administration may be accomplished using a topical cream or ointment or by means of a transdermal patch.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection.

Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### Treatment Methods

In various embodiments, the present invention provides a method of treating a condition in a subject. In various embodiments, method consists of or consists essentially of or comprises: administering a therapeutically effective amount of an inhibitor of GIT1 expression and/or activity to the subject, thereby treating condition in the subject.

In various embodiments, the present invention provides a method of reducing the likelihood of having a condition in a subject. In various embodiments, method consists of or consists essentially of or comprises: administering a therapeutically effective amount of an inhibitor of GIT1 expression and/or activity to the subject, thereby reducing the likelihood of having the condition in the subject.

In various embodiments, the present invention provides a method of reducing the severity of a condition in a subject. In various embodiments, method consists of or consists essentially of or comprises: administering a therapeutically effective amount of an inhibitor of GIT1 expression and/or activity to the subject, thereby reducing the severity of the condition in the subject.

In various embodiments, the present invention provides a method of slowing the progression of a condition in a subject. In various embodiments, method consists of or consists essentially of or comprises: administering a therapeutically effective amount of an inhibitor of GIT1 expression and/or activity to the subject, thereby slowing the progression of the condition in the subject.

In various embodiments, the condition is cancer having an increased expression of GIT1 and MAT2B. In various embodiments, the condition is associated with an increased expression of GIT1 and MAT2B.

In various embodiments, the present invention provides a method of inhibiting GIT1 and/or MAT2B expression in a subject in need thereof, comprising administering a therapeutically effective amount of an inhibitor of GIT1 expression and/or activity to the subject, thereby inhibiting GIT1 and/or MAT2B.

In various embodiments, the subject is suspected of having or has increased expression of GIT1 and/or MAT2B.

In various embodiments, the subject is suspected of having or has a condition caused by the increased expression of GIT1 and/or MAT2B.

In some embodiments, the condition is hepatocellular carcinoma, colorectal cancer, or cholangiocarcinoma. In some embodiments, the condition is a cancer and the cancer comprises a hepatoblastoma, a cholangiocarcinoma, or a colorectal cancer. In various embodiments, the cancer is a cancer having increased expression of GIT1 and/or MAT2B. In various embodiments, the condition is a gastrointestinal disease that comprises of a disease in the liver or the colon. In some embodiments, the condition is a gastrointestinal disease that comprises of cirrhosis, hepatitis, primary biliary cirrhosis, jaundice.

In some embodiments, the condition is fibrosis.

In various embodiments, the method further comprises administering at least one inhibitor that modulates the phosphorylation of GIT1. The inhibitor can be provided in a separate composition or in the same composition as the inhibitor of GIT1 expression and/or activity.

In some embodiments, the method further comprises administering an additional anti-cancer therapy.

The term “anti-cancer therapy” refers to a therapy useful in treating cancer. Examples of anti-cancer therapeutic agents include, but are limited to, e.g., surgery, radiation therapy, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., Herceptin™), anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the embodiments described herein.

The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32 and radioactive isotopes of Lu), chemotherapeutic agents, and toxins such as small molecule toxins or enzymatically active toxins of bacterial, fungal, plant or animal origin, including fragments and/or variants thereof.

As used herein, a “chemotherapeutic agent” is a chemical compound useful in the treatment of cancer. Examples of chemotherapeutic agents include, but are not limited to, alkylating agents such as thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone), a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33:183-186 (1994)); dynemicin, including dynemicin A; bisphosphonates, such as clodronate; an esperamicin, as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™, polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™, paclitaxel (Bristol-Myers Squibb Oncology, Princeton, N.J.), ABRAXANE™, Cremophor-free, albumin-engineered nanoparticle formulation of paclitaxel (American Pharmaceutical Partners, Schaumberg, Ill.), and TAXOTERE™, doxetaxel (Rhone-Poulenc Rorer, Antony, France); chloranbucil; GEMZAR™ gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda, ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb™), inhibitors of PKC-alpha, Raf, H-Ras, EGFR (e.g., erlotinib (Tarceva™)) and VEGF-A that reduce cell proliferation and pharmaceutically acceptable salts, acids or derivatives of any of the above.

In some embodiments, the chemotherapeutic agent is selected from the group consisting of Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); epidermal growth factor; hepatic growth factor; fibroblast growth factor, prolactin; placental lactogen; tumor necrosis factor-alpha and -beta; mullerian-inhibiting substance; mouse gonadotropin-associated peptide; inhibin; activin; vascular endothelial growth factor, integrin; thrombopoietin (TPO); nerve growth factors such as NGF-alpha; platelet-growth factor; transforming growth factors (TGFs) such as TGF-alpha and TGF-beta, insulin-like growth factor-I and -II, erythropoietin (EPO); osteoinductive factors; interferons such as interferon-alpha, -beta and -gamma colony stimulating factors (CSFs) such as macrophage-CSF (M-CSF); granulocyte-macrophage-CSF (GM-CSF); and granulocyte-CSF (G-CSF), interleukins (ILs) such as IL-1, IL-1alpha, IL-2, IL-3, IL-4, IL-5, IL-6, IL-7, IL-8, IL-9, IL-10, IL-11, IL-12; a tumor necrosis factor such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

A “growth inhibitory agent” when used herein refers to a compound or composition which inhibits growth of a cell in vitro and/or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, S-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

In accordance with the invention, the inhibitor can be administered using the appropriate modes of administration, for instance, the modes of administration recommended by the manufacturer or as discussed herein. In accordance with the invention, various routes can be utilized to administer the dual inhibitor of the claimed methods, including but not limited to administering topically, intravascularly, intravenously, intraarterially, intratumorally, intramuscularly, subcutaneously, intraperitoneally, intranasally, or orally.

In some embodiments, the inhibitor is a compound of Formula (I), Formula (II), Formula (III), Formula (IV), Formula (V), Formula (VI), Formula (VII), Formula (VIII), Formula (IX), Formula (X), Formula (XI), or a combination thereof.

In various embodiments, the subject is a human. In some embodiments, the subject in need thereof has or is at risk of having cancer. In some embodiments, the subject in need thereof has or is undergoing chemotherapy and/or irradiation. In other embodiments, the subject in need thereof has or is at risk of having a gastrointestinal disease.

Typical dosages of an effective amount of the GIT1 inhibitor can be in the ranges recommended by the manufacturer where known therapeutic compounds are used, and also as indicated to the skilled artisan by the in vitro responses or in vivo responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models.

In various embodiments, the inhibitor may be administered 1-3 times per day, 1-7 times per week, 1-9 times per month, or more, so as to administer an effective amount of the inhibitor to the subject, where the effective amount is any one or more of the doses described herein. In some embodiments, the inhibitor is administered at about 0.1-0.5, 0.5-5, 5-10, 10-20, 20-50, 50-100, 100-200, 200-300, 300-400, 400-500, 500-600, 600-700, 700-800, 800-900, or 900-1000 μM, or a combination thereof. In various embodiments, the inhibitor of GIT1 is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the inhibitor is administered once, twice, three, or more times.

In various embodiments, a method for inhibiting cancer cell growth consists of, consists essentially of, or comprises contacting a cancer cell with an inhibitor of GIT1 expression and/or activity and culturing under conditions and for a sufficient time to promote restriction of cancer cell growth. In some embodiments, the contacting a cancer cell can be in vitro. In other embodiments, the contacting a cancer cell can be in vivo. In various embodiments, the cancer cell originates from the liver or the colon. In some embodiments, the cancer cell is a hepatoblastoma, cholangiocarcinoma, a colorectal carcinoma, carcinomas of the breast, lung cancer, oral squamous carcinoma, stomach cancer, or a cancer having an increased expression of GIT1 and MAT2B.

In various embodiments, the inhibitor of GIT1 expression and/or activity comprises a small molecule. In various embodiments, the inhibitor of GIT1 expression and/or activity is a compound of Formula (I), Formula (II), Formula (III), Formula (IV), Formula (V), Formula (VI), Formula (VII), Formula (VIII), Formula (IX), Formula (X), Formula (XI), or a combination thereof.

In various embodiments, the inhibitor of GIT1 expression and/or activity is a compound of Formula (A), Formula (A-1), Formula (A-2), Formula (A-3), Formula (A-4), or a combination thereof.

In some embodiments, the inhibitor of GIT1 expression and/or activity is not compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

In some embodiments, the inhibitor of GIT1 expression and/or activity is compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

### Kits of the Invention

In various embodiments, the present invention provides a kit for treating, preventing, or reducing the likelihood of having, reducing the severity of and/or slowing the progression of a condition in a subject. The kit consists of, consists essentially of, or comprises: a GIT1 inhibitor of expression and/or activity; and instructions for using the GIT1 inhibitor to treat, prevent, reduce the likelihood of having, reduce the severity of and/or slow the progression of the condition in the subject.

In some embodiments, the inhibitor is a compound of Formula (I), Formula (II), Formula (III), Formula (IV), Formula (V), Formula (VI), Formula (VII), Formula (VIII), Formula (IX), Formula (X), Formula (XI), or a combination thereof.

In some embodiments, the inhibitor is a compound of Formula (A), Formula (A-1), Formula (A-2), Formula (A-3), Formula (A-4), or a combination thereof.

In some embodiments, the inhibitor is not compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

In some embodiments, the inhibitor is compound C3 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

In some embodiments, the kit further comprises a device to administer the GIT1 inhibitor to a subject in need thereof.

### VARIOUS NON-LIMITING EMBODIMENTS OF THE INVENTION

Embodiments include those listed below.

Embodiment 1. A compound of Formula (I):

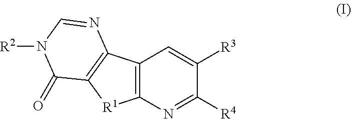

wherein:
R₁ is an amine, sulfur, oxygen, C(CH₃)₂, or methylene, each of which can be optionally substituted;
R₂ is a hydrogen, carbonyl, aromatic moiety, alkyl, acyl, cyclyl or heterocyclyl, each of which can be optionally substituted;
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

Embodiment 2. The compound of embodiment 1, having the structure of Formula (II):

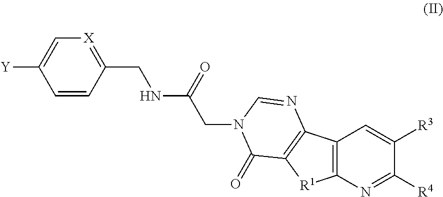

wherein:
X is a nitrogen or CH, which can be optionally substituted;
Y is a hydrogen, halide, nitrile, alkyl, acyl, or hydroxyl, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and

R₃ and R₁ can be joined together by either three or four methylene groups, each of which can be optionally substituted, or replaced by an oxygen or nitrogen atom.

Embodiment 3. The compound of embodiment 1, having the structure of Formula (III):

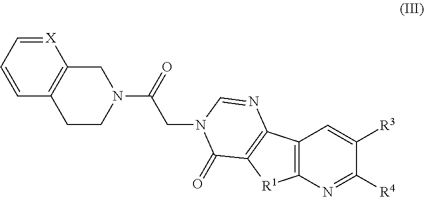

wherein:
X is methine or nitrogen;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

Embodiment 4. The compound of embodiment 1, having the structure of Formula (IV):

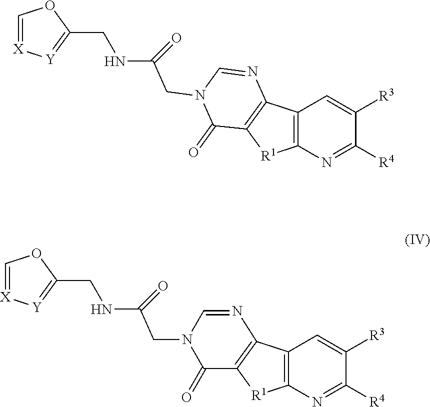

wherein:
X is methine or nitrogen;
Y is methine or nitrogen;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

Embodiment 5. The compound of embodiment 1, having the structure of Formula (V):

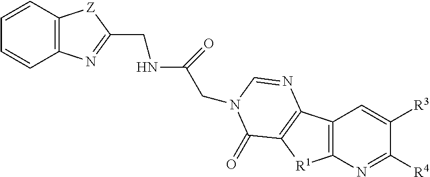

wherein:
Z is an oxygen or sulfur, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

Embodiment 6. The compound of embodiment 1, having the structure of Formula (VI):

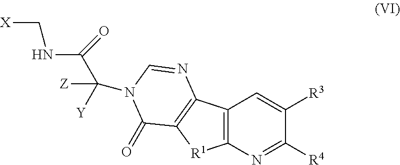

X is an aromatic moiety, alkyl, acyl, heteroaryl, cyclyl or heterocyclyl, each of which can be optionally substituted;
Y and/or Z is a hydrogen, aromatic moiety, alkyl, acyl, cyclyl or heterocyclyl, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

Embodiment 7. The compound of embodiment 1, having the structure of Formula (VII):

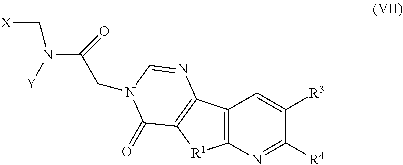

wherein:
X is an aromatic moiety, alkyl, acyl, cyclyl or heterocyclyl, each of which can be optionally substituted;
Y is an aromatic moiety, alkyl, acyl, cyclyl or heterocyclyl, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

Embodiment 8. The compound of embodiment 1, having the structure of Formula (VIII):

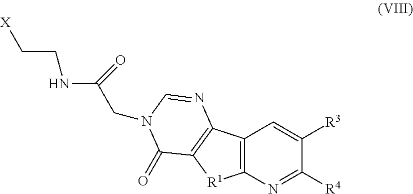

wherein;
X is an aromatic moiety, alkyl, acyl, cyclyl or heterocyclyl, aryl, or heteroaryl, each of which can be optionally substituted;
R₁ is an amine, sulfur, oxygen, C(CH₃)₂, or methylene, each of which can be optionally substituted; and
R₃ is a hydrogen, alcohol, aromatic moiety, alkyl, acyl, cyclyl or heterocyclyl, each of which can be optionally substituted;
R₄ is a hydrogen, alcohol, aromatic moiety, alkyl, acyl, cyclyl or heterocyclyl, each of which can be optionally substituted; and
R₃ and R₄ can be joined together by either three or four methylene groups, each of which can be optionally substituted, or replaced by an oxygen or nitrogen atom.

Embodiment 9. The compound of embodiment 1, having the structure of Formula (IX):

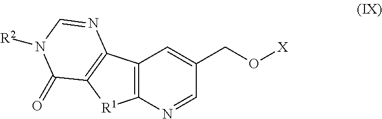

wherein:
X is a hydrogen, lower alkyl, O(CH₂)₂OH, or alcohol, each of which can be optionally substituted;
R₁ is an amine, sulfur, oxygen, C(CH₃)₂, or methylene, each of which can be optionally substituted; and
R₂ is a hydrogen, carbonyl, aromatic moiety, alkyl, acyl, cyclyl or heterocyclyl, each of which can be optionally substituted.

Embodiment 10. The compound of embodiment 1, having the structure of Formula (X):

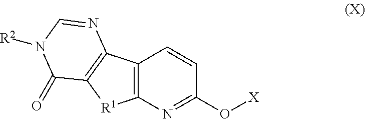

wherein:
X is a hydrogen, lower alkyl, O(CH₂) OH, or alcohol, each of which can be optionally substituted;
R₁ is an amine, sulfur, oxygen, C(CH₃)₂, or methylene, each of which can be optionally substituted; and
R₂ is a hydrogen, carbonyl, aromatic moiety, alkyl, acyl, cyclyl or heterocyclyl, each of which can be optionally substituted.

Embodiment 11. The compound of Formula (XI):

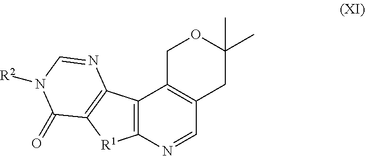

wherein:
R₁ is an amine, sulfur, oxygen, C(CH₃)₂, or methylene, each of which can be optionally substituted; and
R₂ is a hydrogen, carbonyl, aromatic moiety, alkyl, acyl, cyclyl or heterocyclyl, each of which can be optionally substituted.

Embodiment 12. The compound of embodiment 1, wherein the compound is a pharmaceutically acceptable salt, solvate, hydrate, polymorph, co-crystal, tautomer, stereoisomer, or isotopically labeled derivative thereof.

Embodiment 13. A pharmaceutical composition comprising an inhibitor of GIT1 expression and/or activity.

Embodiment 14. The pharmaceutical composition of embodiment 13, wherein the inhibitor is any one of embodiments 1-11.

Embodiment 15. The pharmaceutical composition of embodiment 13, further comprising a pharmaceutically acceptable carrier or excipient.

Embodiment 16. The pharmaceutical composition of embodiment 13, wherein the inhibitor of GIT1 inhibits contacting between GIT1 and its normal interacting proteins.

Embodiment 17. The method of embodiment 16, wherein GIT1's normal interacting protein is MAT2B.

Embodiment 18. The pharmaceutical composition of embodiment 13, wherein the inhibitor of GIT1 inhibits contacting between GIT1 and MEK1, MEK2, and recruitment of cRAF, BRAF, and ERK to MEK1/2.

Embodiment 19. The pharmaceutical composition of embodiment 13, wherein the inhibitor of GIT1 promotes contacting between GIT1 and cyclin B1.

Embodiment 20. The pharmaceutical composition of embodiment 13, wherein the inhibitor of GIT1 promotes contacting between cyclin B1 and CDK1.

Embodiment 21. The pharmaceutical composition of embodiment 13, wherein the composition is formulated for topical, intravascular, intravenous, intraarterial, intratumoral, intramuscular, subcutaneous, intraperitoneal, intranasal, or oral administration.

Embodiment 22. A method of treating, preventing, reducing the likelihood of having, reducing the severity of and/or slowing the progression of a condition in a subject comprising:
administering a therapeutically effective amount of an inhibitor of GIT1 expression and/or activity to the subject, thereby treating, preventing, reducing the likelihood of having, reducing the severity of and/or slowing the progression of the condition in the subject,
wherein the condition is cancer or a condition associated with an increased expression of GIT1 and MAT2B.

Embodiment 23. The method of embodiment 22, wherein the condition is hepatocellular carcinoma, colorectal cancer, or cholangiocarcinoma.

Embodiment 24. The method of embodiment 22, wherein the subject is a human.

Embodiment 25. The method of embodiment 22, wherein the inhibitor of GIT1 is administered topically, intravascularly, intravenously, intraarterially, intratumorally, intramuscularly, subcutaneously, intraperitoneally, intranasally, or orally.

Embodiment 26. The method of embodiment 22, wherein the inhibitor of GIT1 is a compound of any one of embodiments 1-11.

Embodiment 27. The method of embodiment 22, further comprising a step of administering or contacting with at least one inhibitor that modulates the phosphorylation of GIT1.

Embodiment 28. The method of embodiment 22, further comprising administering an additional anti-cancer therapy.

Embodiment 29. The method of embodiment 22, wherein the subject in need thereof has or is at risk of having cancer.

Embodiment 30. The method of embodiment 22, wherein the subject in need thereof has or is undergoing chemotherapy and/or irradiation.

Embodiment 31. The method of embodiment 22, wherein the cancer comprises a hepatoblastoma, a cholangiocarcinoma, a colorectal cancer, carcinomas of the breast, lung cancer, oral squamous carcinoma, stomach cancer, or a cancer having an increased expression of GIT1 and MAT2B.

Embodiment 32. The method of embodiment 22, wherein the subject in need thereof has or is at risk of having a gastrointestinal disease.

Embodiment 33. The method of embodiment 32, wherein the gastrointestinal disease comprises of a disease in the liver or the colon.

Embodiment 34. The method of embodiment 32, wherein the gastrointestinal disease comprises of Crohn's Disease, ulcerative colitis, cirrhosis, hemochromatosis, Wilson's disease, autoimmune hepatitis, primary biliary cirrhosis, jaundice, diverticulitis, gastritis, chronic diarrhea, and necrotizing enterocolitis.

Embodiment 35. The method of any one of embodiments 22-34, wherein the subject in need thereof is human.

Embodiment 36. A kit for treating, preventing, or reducing the likelihood of having, reducing the severity of and/or slowing the progression of a condition in a subject, comprising: a GIT1 inhibitor of expression and/or activity; and instructions for using the GIT1 inhibitor to treat, prevent, reduce the likelihood of having, reduce the severity of and/or slow the progression of the condition in the subject.

Embodiment 37. The kit of embodiment 36, wherein the GIT1 inhibitor is any one of claims 1-11.

Embodiment 38. The kit of embodiment 36, further comprising a device to administer the GIT1 inhibitor to a subject in need thereof.

Embodiments include those listed below.

In various embodiments, Compound C3 is PubChem Compound ID #663383 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide), having the structure:

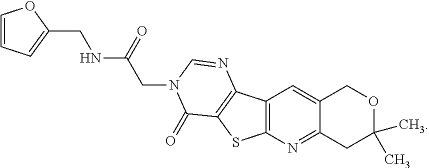 [SMILES: CC1(C)Cc2nc3sc4c(=O)n(CC(=O)NCc5ccco5)cnc4c3cc2CO1]

MST K_{D} 6.2 μM. RKO/HepG2 IC₅₀ ca. 20 μM. MW 424. PSA 93. c Log P 1.3.

Embodiments include those listed below.

Embodiment 1A. A compound of Formula (A):

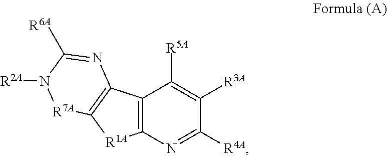

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{3A} is H, halo, optionally substituted alkyl, or OR^{9A}, where R^{9A} is H or optionally substituted alkyl;
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl; and
R^{A} is C═O or CH₂; or
R^{3A} and R^{4A} can be linked together to form a ring, wherein the ring is optionally substituted; or
R^{3A} and R^{5A} can be linked together to form a ring, wherein the ring is optionally substituted, with the proviso that the compound of Formula (A) is not

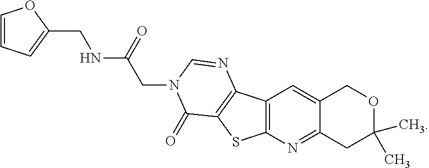 [SMILES: CC1(C)Cc2nc3sc4c(=O)n(CC(=O)NCc5ccco5)cnc4c3cc2CO1]

Embodiment 2A. The compound of embodiment 1A, wherein R^{2A} is:

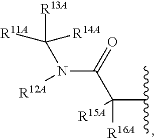

wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl.

Embodiment 3A. The compound of embodiment 2A, wherein R^{2A} is:

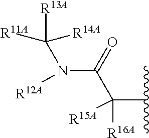

wherein:
R^{11A} is H, halo, optionally substituted alkyl, optionally substituted aryl, optionally substituted heteroaryl, optionally substituted cyclyl, optionally substituted heterocyclyl;
R^{12A} is H, CH₃, or CH(CH₃)₂;
R^{13A} is H, CH₃, or CH(CH₃)₂;
R^{14A} is H, CH₃, or CH(CH₃)₂;
R^{15A} is H, CH₃, or CH(CH₃); and
R^{16A} is H, CH₃, or CH(CH₃)₂.

Embodiment 4A. The compound of embodiment 2A, wherein R^{2A} is:

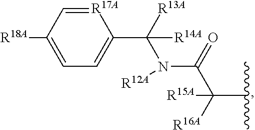

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{12A} is N or CH; and
R^{18A} is H or halo.

Embodiment 5A. The compound of embodiment 4A, wherein R^{2A} is:

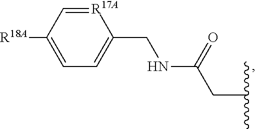

wherein:
R^{17A} is N or CH; and
R^{18A} is H or F.

Embodiment 6A. The compound of embodiment 2A, wherein R^{2A} is:

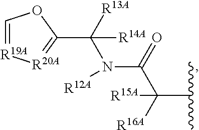

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl;
R^{19A} is N or CH; and
R^{20A} is N or CH.

Embodiment 7A. The compound of embodiment 6A, wherein R^{2A} is:

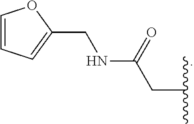 [SMILES: *CC(=O)NCc1ccco1]

Embodiment 8A. The compound of embodiment 2A, wherein R^{2A} is:

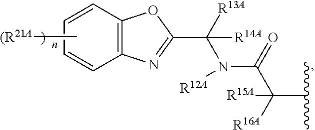

wherein:
R^{12A} is H or optionally substituted alkyl;
R^{13A} is H, halo, or optionally substituted alkyl;
R^{14A} is H, halo, or optionally substituted alkyl,
R^{15A} is H, halo, or optionally substituted alkyl;
R^{16A} is H, halo, or optionally substituted alkyl;
R^{21A} is independently H, halo, or optionally substituted alkyl; and
n is 1, 2 3, or 4.

Embodiment 9A. The compound of embodiment 8A, wherein R^{2A} is:

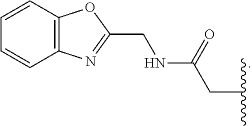 [SMILES: *CC(=O)NCc1nc2ccccc2o1]

Embodiment 10A. The compound of embodiment 1A, wherein R^{2A} is:

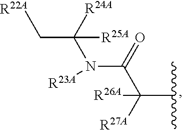

wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl

Embodiment 11A. The compound of embodiment 10A, wherein R^{2A} is:

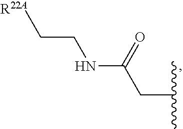

wherein:
R^{22A} is:

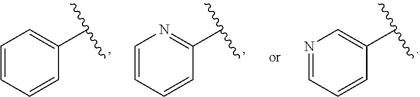 [SMILES: *c1ccccc1 ; *c1ccccn1 ; *c1cccnc1]

Embodiment 12A. The compound of embodiment 1A, wherein R^{2A} is:

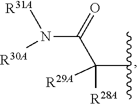

wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted.

Embodiment 13A. The compound of embodiment 12A, wherein R^{2A} is:

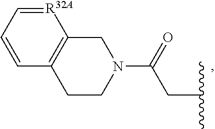

wherein: R^{32A} is CH or N.

Embodiment 14A. The compound of embodiment 12A, wherein R^{2A} is;

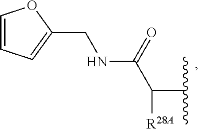

wherein: R^{28A} is H or CH₃; or

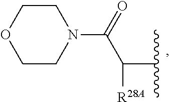

wherein: R^{28A} is H or CH₃;

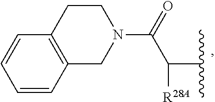

wherein: R^{28A} is H or CH₃; or

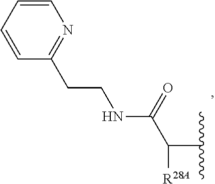

wherein: R^{28A} is H or CH₃.

Embodiment 15A. The compound of embodiment 1A, wherein R^{2A} is:

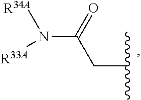

wherein,
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

Embodiment 16A. The compound of embodiment 15A, wherein R^{2A} is:

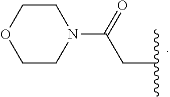 [SMILES: *CC(=O)N1CCOCC1]

Embodiment 17A. The compound of embodiment 15A, wherein R^{2A} is:

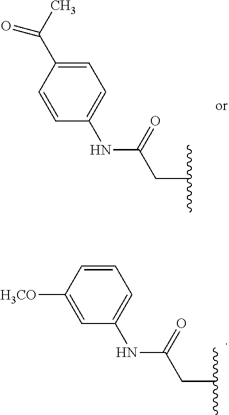 [SMILES: *CC(=O)Nc1ccc(C(C)=O)cc1 ; *CC(=O)Nc1cccc(OC)c1]

Embodiment 18A. The compound of embodiment 1A, having a structure of Formula (A-1):

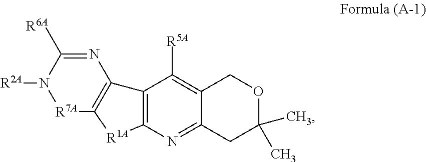

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted
R^{5A} is H, halo, or optionally substituted alkyl;
R^{6A} is H, halo, or optionally substituted alkyl; and
R^{7A} is C═O or CH₂.

Embodiment 19A. The compound of embodiment 18A, wherein R^{2A} is:

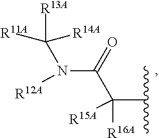

wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl.

Embodiment 20A. The compound of embodiment 19A, wherein R^{2A} is:

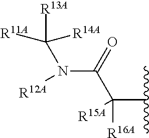

R^{11A} is H, halo, optionally substituted alkyl, optionally substituted aryl, optionally substituted heteroaryl, optionally substituted cyclyl, optionally substituted heterocyclyl;
R^{12A} is H, CH₃, or CH(CH₃)₂;
R^{13A} is H, CH₃, or CH(CH₃)₂;
R^{14A} is H, CH₃, or CH(CH₃)₂;
R^{15A} is H, CH₃, or CH(CH₃)₂; and
R^{16A} is H, CH₃, or CH(CH₃)₂.

Embodiment 21A. The compound of embodiment 19A, wherein R^{2A} is:

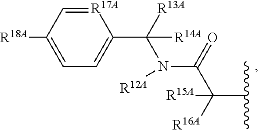

wherein:
R^{12A} is H or optionally substituted alkyl;
R^{1A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{12A} is N or CH; and
R^{15A} is H or halo.

Embodiment 22A. The compound of embodiment 21A, wherein R^{2A} is:

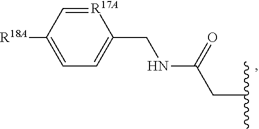

wherein:
R^{17A} is Nor CH; and
R^{18A} is H or F.

Embodiment 23A. The compound of embodiment 19A, wherein R^{2A} is;

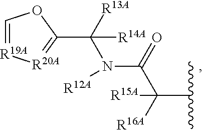

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl, and
R^{16A} is H, halo, or optionally substituted alkyl;
R^{19A} is N or CH; and
R^{20A} is N or CH.

Embodiment 24A. The compound of embodiment 23A, wherein R^{2A} is:

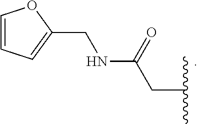 [SMILES: *CC(=O)NCc1ccco1]

Embodiment 25A. The compound of embodiment 19A, wherein R^{2A} is:

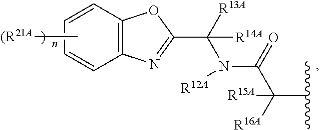

wherein:
R^{12A} is H or optionally substituted alkyl;
R^{1A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{21A} is independently H, halo, or optionally substituted alkyl; and
n is 1, 2 3, or 4.

Embodiment 26A. The compound of embodiment 25A, wherein R^{2A} is:

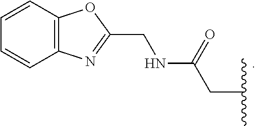 [SMILES: *CC(=O)NCc1nc2ccccc2o1]

Embodiment 27A. The compound of embodiment 18A, wherein R^{2A} is:

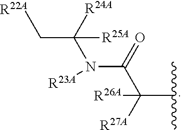

wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl,
R^{25A} is H, halo, or optionally substituted alkyl;
R^{26A} is H, halo, or optionally substituted alkyl; and
R^{27A} is H, halo, or optionally substituted alkyl.

Embodiment 28A. The compound of embodiment 27A, wherein R^{2A} is:

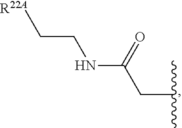

wherein:
R^{22A} is:

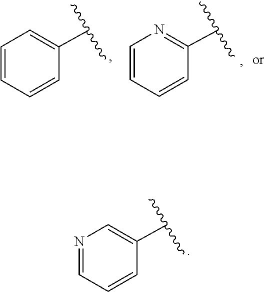

Embodiment 29A. The compound of embodiment 18A, wherein R^{2A} is:

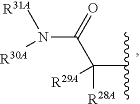

wherein:
R^{28A} is H, halo, or optionally substituted alkyl;
R^{29A} is H, halo, or optionally substituted alkyl;
R^{30A} is H or optionally substituted alkyl; and
R₃IA is H or optionally substituted alkyl; or
R^{30A} and R^{31A} can be linked together to form a ring, wherein the ring is optionally substituted.

Embodiment 30A. The compound of embodiment 29A, wherein R^{2A} is:

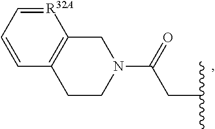

wherein: R^{32A} is CH or N.

Embodiment 31A. The compound of embodiment 29A, wherein R^{2A} is:

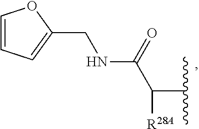

wherein: R^{28A} is H or CH₃; or

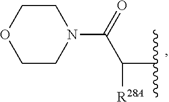

wherein: R^{28A} is H or CH₃;

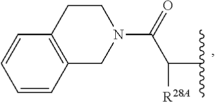

wherein: R^{28A} is H or CH₃; or

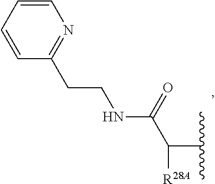

wherein: R^{28A} is H or CH₃.

Embodiment 32A. The compound of embodiment 18A, wherein R^{2A} is:

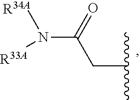

wherein,
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

Embodiment 33A. The compound of embodiment 32A, wherein R^{2A} is:

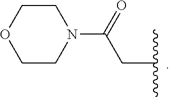 [SMILES: *CC(=O)N1CCOCC1]

Embodiment 34A. The compound of embodiment 33A, wherein R^{2A} is:

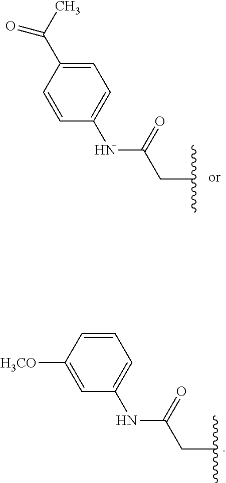 [SMILES: *CC(=O)Nc1ccc(C(C)=O)cc1 ; *CC(=O)Nc1cccc(OC)c1]

Embodiment 35A. The compound of embodiment 1A, having a structure of Formula (A-2):

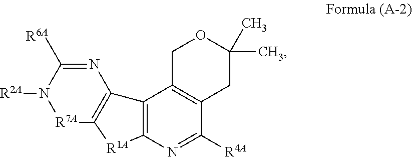

R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted
R^{4A} is H, halo, optionally substituted alkyl, or OR^{10A}, where R^{10A} is H or optionally substituted alkyl;
R^{6A} is H, halo, or optionally substituted alkyl; and
R^{7A} is C═O or CH₂.

Embodiment 36A. The compound of embodiment 35A, wherein R^{2A} is:

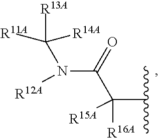

wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl.

Embodiment 37A. The compound of embodiment 36A, wherein R^{2A} is:

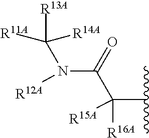

R^{11A} is H, halo, optionally substituted alkyl, optionally substituted aryl, optionally substituted heteroaryl, optionally substituted cyclyl, optionally substituted heterocyclyl;
R^{12A} is H, CH₃, or CH(CH₃)₂;
R^{13A} is H, CH₃, or CH(CH₃)₂;
R^{14A} is H, CH₃, or CH(CH₃)₂;
R^{15A} is H, CH₃, or CH(CH₃)₂; and
R^{16A} is H, CH₃, or CH(CH₃)₂.

Embodiment 38A. The compound of embodiment 36A, wherein R^{2A} is:

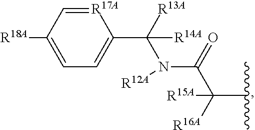

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{12A} is N or CH; and
R^{18A} is H or halo.

Embodiment 39A. The compound of embodiment 38A, wherein R^{2A} is:

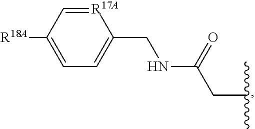

wherein:
R^{17A} is N or CH; and
R^{18A} is H or F.

Embodiment 40A. The compound of embodiment 36A, wherein R^{2A} is:

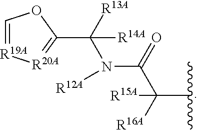

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl;
R^{19A} is N or CH; and
R^{20A} is N or CH.

Embodiment 41A. The compound of embodiment 40A, wherein R^{2A} is:

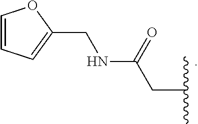 [SMILES: *CC(=O)NCc1ccco1]

Embodiment 42A. The compound of embodiment 36A, wherein R^{2A} is:

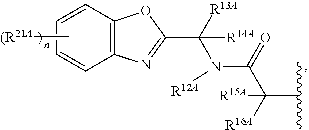

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{21A} is independently H, halo, or optionally substituted alkyl; and
n is 1, 2 3, or 4.

Embodiment 43A. The compound of embodiment 42A, wherein R^{2A} is:

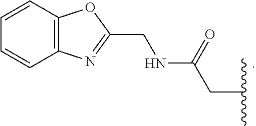 [SMILES: *CC(=O)NCc1nc2ccccc2o1]

Embodiment 44A. The compound of embodiment 35A, wherein R^{2A} is:

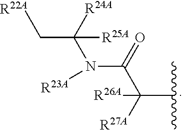

wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl.

Embodiment 45A. The compound of embodiment 44A, wherein R^{2A} is:

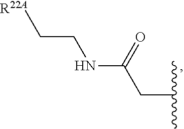

wherein:
R^{22A} is:

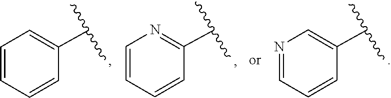

Embodiment 46A. The compound of embodiment 35A, wherein R^{2A} is:

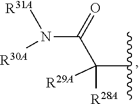

wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted.

Embodiment 47A. The compound of embodiment 46A, wherein R^{2A} is:

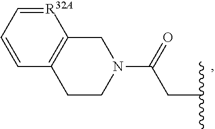

wherein: R^{32A} is CH or N.

Embodiment 48A. The compound of embodiment 46A, wherein R^{2A} is:

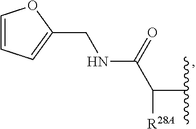

wherein: R^{28A} is H or CH₃; or

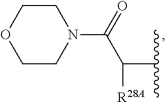

wherein: R^{28A} is H or CH₃;

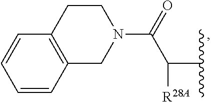

wherein: R^{28A} is H or CH₃; or

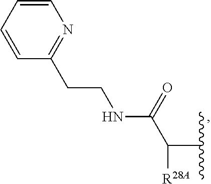

wherein: R^{28A} is H or CH₃.

Embodiment 49A. The compound of embodiment 35A, wherein R^{2A} is:

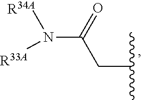

wherein:
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

Embodiment 50A. The compound of embodiment 49A, wherein R^{2A} is:

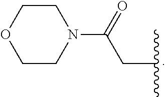 [SMILES: *CC(=O)N1CCOCC1]

Embodiment 51A. The compound of embodiment 49A, wherein R^{2A} is:

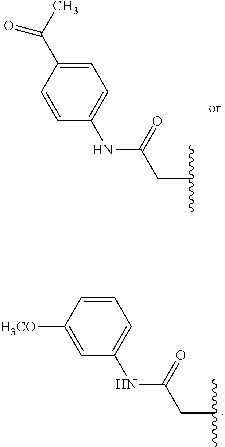 [SMILES: *CC(=O)Nc1ccc(C(C)=O)cc1 ; *CC(=O)Nc1cccc(OC)c1]

Embodiment 52A. The compound of embodiment 1A, having a structure of Formula (A-3):

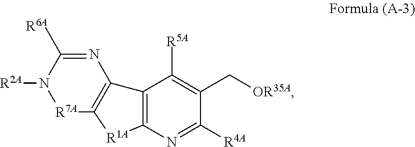

wherein:
R^{1A} is S, O, CH₂, C(CH₂)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl;
R^{7A} is C═O or CH₂; and
R^{35A} is H, CH₃, or CH CH₂OH.

Embodiment 53A. The compound of embodiment 52A, wherein R^{2A} is:

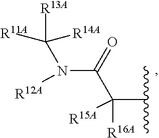

wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl, and
R^{16A} is H, halo, or optionally substituted alkyl.

Embodiment 54A. The compound of embodiment 53A, wherein R^{2A} is:

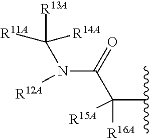

wherein:
R^{11A} is H, halo, optionally substituted alkyl, optionally substituted aryl, optionally substituted heteroaryl, optionally substituted cyclyl, optionally substituted heterocyclyl;
R^{12A} is H, CH₃, or CH(CH₃)₂;
R^{13A} is H, CH₃, or CH(CH₃)₂;
R^{14A} is H, CH₃, or CH(CH₃)₂;
R^{15A} is H, CH₃, or CH(CH₃)₂; and
R^{16A} is H, CH₃, or CH(CH₃)₂.

Embodiment 55A. The compound of embodiment 53A, wherein R^{2A} is:

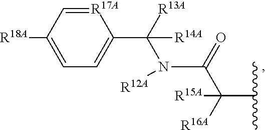

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{12A} is N or CH; and
R^{18A} is H or halo.

Embodiment 56A. The compound of embodiment 55A, wherein R^{2A} is:

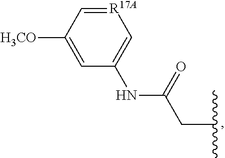

wherein:
R^{17A} is N or CH; and
R^{15A} is H or F.

Embodiment 57A. The compound of embodiment 53A, wherein R^{2A} is:

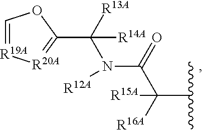

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl, and
R^{16A} is H, halo, or optionally substituted alkyl;
R^{19A} is N or CH; and
R^{20A} is N or CH.

Embodiment 58A. The compound of embodiment 57A, wherein R^{2A} is:

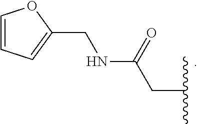 [SMILES: *CC(=O)NCc1ccco1]

Embodiment 59A. The compound of embodiment 53A, wherein R^{2A} is:

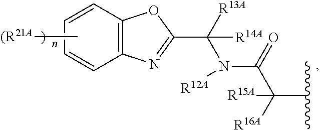

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{21A} is independently H, halo, or optionally substituted alkyl; and
n is 1, 2 3, or 4.

Embodiment 60A. The compound of embodiment 59A, wherein R^{2A} is:

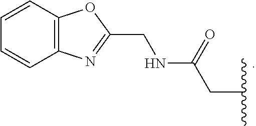 [SMILES: *CC(=O)NCc1nc2ccccc2o1]

Embodiment 61A. The compound of embodiment 52A, wherein R^{2A} is:

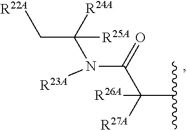

wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl.

Embodiment 62A. The compound of embodiment 61A, wherein R^{2A} is:

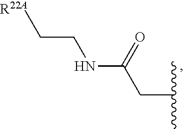

wherein:
R^{22A} is:

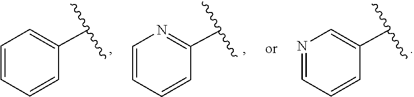 [SMILES: *c1ccccc1 ; *c1ccccn1 ; *c1cccnc1]

Embodiment 63A. The compound of embodiment 52A, wherein R^{2A} is:

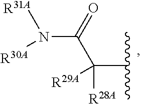

wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted.

Embodiment 64A. The compound of embodiment 63A, wherein R^{2A} is:

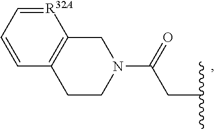

wherein: R^{32A} is CH or N.

Embodiment 65A. The compound of embodiment 63A, wherein R^{2A} is:

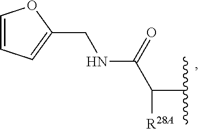

wherein: R^{28A} is H or CH₃; or

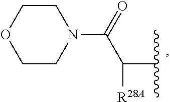

wherein: R^{28A} is H or CH₃;

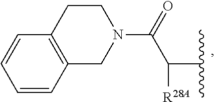

wherein: R^{28A} is H or CH₃; or

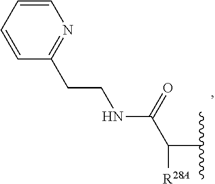

wherein: R^{28A} is H or CH₃.

Embodiment 66A. The compound of embodiment 52A, wherein R^{2A} is:

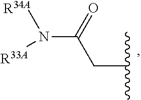

wherein,
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

Embodiment 67A. The compound of embodiment 66A, wherein R^{2A} is:

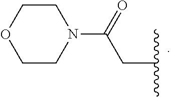 [SMILES: *CC(=O)N1CCOCC1]

Embodiment 68A. The compound of embodiment 66A, wherein R^{2A} is:

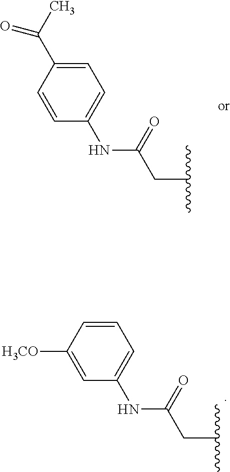 [SMILES: *CC(=O)Nc1ccc(C(C)=O)cc1 ; *CC(=O)Nc1cccc(OC)c1]

Embodiment 69A. The compound of embodiment 1A, having a structure of Formula (A-4):

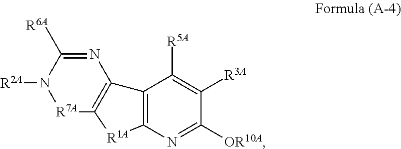

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted
R^{3A} is H, halo, optionally substituted alkyl, or OR^{9A}, where R^{9A} is H or optionally substituted alkyl;
R^{5A} is H, halo, or optionally substituted alkyl;
R^{6A} is H, halo, or optionally substituted alkyl;
R^{7A} is C═O or CH₂; and
R^{10A} is H, CH₃, or CH CH₂OH.

Embodiment 70A. The compound of embodiment 69A, wherein R^{2A} is:

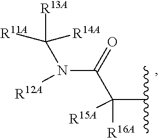

wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl, and
R^{16A} is H, halo, or optionally substituted alkyl.

Embodiment 71A. The compound of embodiment 70A, wherein R^{2A} is:

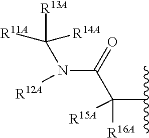

wherein:
R^{11A} is H, halo, optionally substituted alkyl, optionally substituted aryl, optionally substituted heteroaryl, optionally substituted cyclyl, optionally substituted heterocyclyl;
R^{12A} is H, CH₃, or CH(CH₃)₂,
R^{13A} is H, CH₃, or CH(CH₃)₂;
R^{14A} is H, CH₃, or CH(CH₃)₂;
R^{15A} is H, CH₃, or CH(CH₃)₂; and
R^{16A} is H, CH₃, or CH(CH₃)₂.

Embodiment 72A. The compound of embodiment 70A, wherein R^{2A} is:

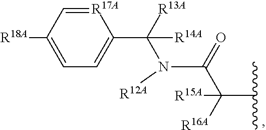

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{12A} is N or CH; and
R^{18A} is H or halo.

Embodiment 73A. The compound of embodiment 72A, wherein R^{2A} is:

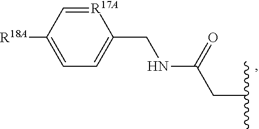

wherein:
R^{17A} is N or CH; and
R^{18A} is H or F.

Embodiment 74A. The compound of embodiment 70A, wherein R^{2A} is:

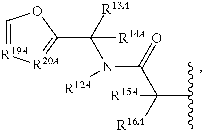

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl, and
R^{16A} is H, halo, or optionally substituted alkyl;
R^{19A} is N or CH; and
R^{20A} is N or CH.

Embodiment 75A. The compound of embodiment 74A, wherein R^{2A} is:

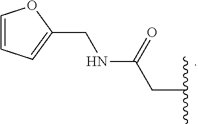 [SMILES: *CC(=O)NCc1ccco1]

Embodiment 76A. The compound of embodiment 70A, wherein R^{2A} is:

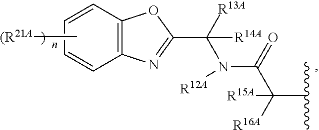

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{21A} is independently H, halo, or optionally substituted alkyl; and
n is 1, 2 3, or 4.

Embodiment 77A. The compound of embodiment 76A, wherein R^{2A} is:

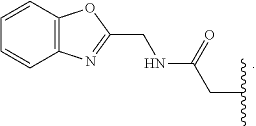 [SMILES: *CC(=O)NCc1nc2ccccc2o1]

Embodiment 78A. The compound of embodiment 69A, wherein R^{2A} is:

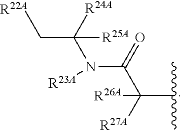

wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl.

Embodiment 79A. The compound of embodiment 78A, wherein R^{2A} is:

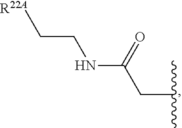

wherein:
R^{22A} is:

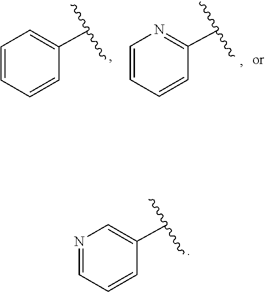

Embodiment 80A. The compound of embodiment 69A, wherein R^{2A} is:

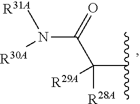

wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted.

Embodiment 81A. The compound of embodiment 78A, wherein R^{2A} is:

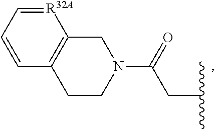

wherein: R^{32A} is CH or N.

Embodiment 82A. The compound of embodiment 80A, wherein R^{2A} is:

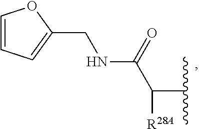

wherein: R^{28A} is H or CH₃; or

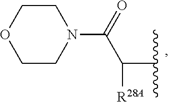

wherein: R^{28A} is H or CH₃;

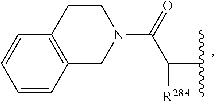

wherein: R^{28A} is H or CH₃; or

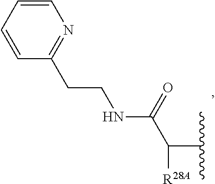

wherein: R^{28A} is H or CH₃.

Embodiment 83A. The compound of embodiment 69A, wherein R^{2A} is:

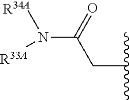

R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

Embodiment 84A. The compound of embodiment 83A, wherein R^{2A} is:

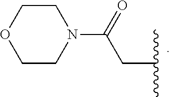 [SMILES: *CC(=O)N1CCOCC1]

Embodiment 85A. The compound of embodiment 83A, wherein R^{2A} is:

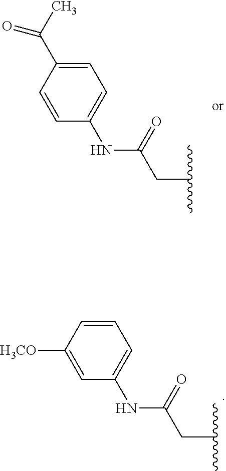 [SMILES: *CC(=O)Nc1ccc(C(C)=O)cc1 ; *CC(=O)Nc1cccc(OC)c1]

Embodiment 86A. The compound of embodiment 1A, selected from the group consisting of:

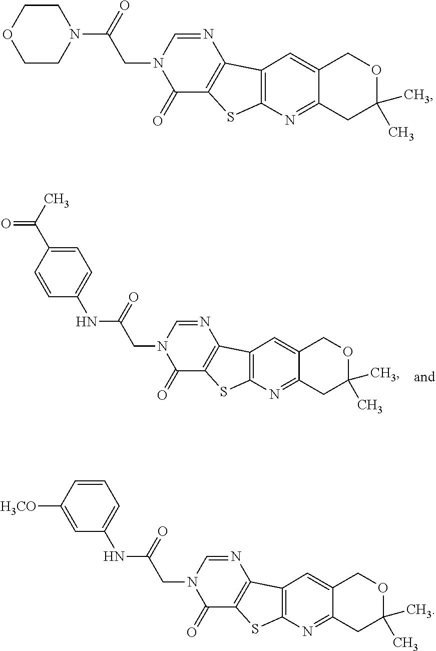 [SMILES: CC1(C)Cc2nc3sc4c(=O)n(CC(=O)N5CCOCC5)cnc4c3cc2CO1 ; CC(=O)c1ccc(NC(=O)Cn2cnc3c(sc4nc5c(cc43)COC(C)(C)C5)c2=O)cc1 ; COc1cccc(NC(=O)Cn2cnc3c(sc4nc5c(cc43)COC(C)(C)C5)c2=O)c1]

Embodiment 87A. The compound of embodiment 1A, wherein the compound is:

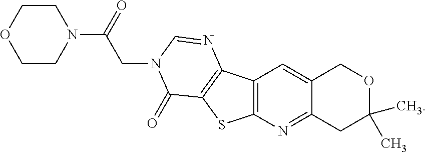 [SMILES: CC1(C)Cc2nc3sc4c(=O)n(CC(=O)N5CCOCC5)cnc4c3cc2CO1]

Embodiment 88A. The compound of embodiment 1A, wherein the compound is:

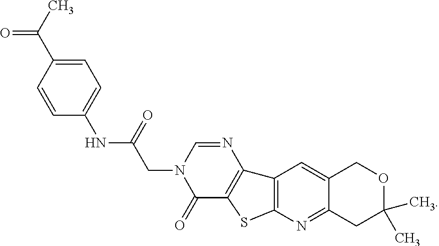 [SMILES: CC(=O)c1ccc(NC(=O)Cn2cnc3c(sc4nc5c(cc43)COC(C)(C)C5)c2=O)cc1]

Embodiment 89A. The compound of embodiment IA, wherein the compound is:

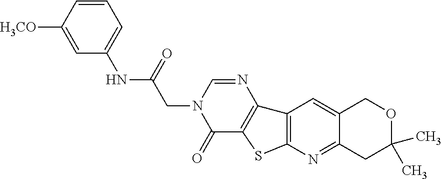 [SMILES: COc1cccc(NC(=O)Cn2cnc3c(sc4nc5c(cc43)COC(C)(C)C5)c2=O)c1]

Embodiment 90A. The compound of any one of embodiments 1A-89A, wherein the compound is a pharmaceutically acceptable salt, solvate, hydrate, polymorph, co-crystal, tautomer, stereoisomer, or isotopically labeled derivative thereof.

Embodiment 91A. A pharmaceutical composition comprising an inhibitor of GIT1's normal interactome.

Embodiment 92A. The pharmaceutical composition of embodiment 91A, wherein the inhibitor of GIT1 is a compound of any one of embodiments 1A-90A.

Embodiment 93A. The pharmaceutical composition of embodiment 91A, further comprising a pharmaceutically acceptable carrier or excipient.

Embodiment 94A. The pharmaceutical composition of embodiment 91A, wherein the inhibitor of GIT1 inhibits contact between GIT1 and its normal interacting proteins.

Embodiment 95A. The pharmaceutical composition of embodiment 94A, wherein one of GIT1's normal interacting proteins is MAT2B.

Embodiment 96A. The pharmaceutical composition of embodiment 91A, wherein the inhibitor of GIT1 inhibits interaction between GIT1 and MEK1, MEK2, and recruitment of cRAF, BRAF, and ERK to MEK1/2.

Embodiment 97A. The pharmaceutical composition of embodiment 91A, wherein the inhibitor of GIT1 promotes contact between GIT1 and cyclin B1.

Embodiment 98A. The pharmaceutical composition of embodiment 91A, wherein the inhibitor of GIT1 promotes contact between cyclin B1 and CDK1.

Embodiment 99A. The pharmaceutical composition of embodiment 91A, wherein the composition is formulated for topical, intravascular, intravenous, intraarterial, intratumoral, intramuscular, subcutaneous, intraperitoneal, intranasal, or oral administration.

Embodiment 100A. The pharmaceutical composition of embodiment 91A, wherein the inhibitor of GIT1 is:

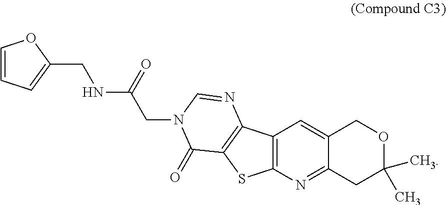 [SMILES: CC1(C)Cc2nc3sc4c(=O)n(CC(=O)NCc5ccco5)cnc4c3cc2CO1]

Embodiment 101A. A method of treating, preventing, reducing the likelihood of having, reducing the severity of and/or slowing the progression of a condition in a subject comprising:

administering a therapeutically effective amount of an inhibitor of GIT1's normal interactome to the subject, thereby treating, preventing, reducing the likelihood of having, reducing the severity of and/or slowing the progression of the condition in the subject, wherein the condition is cancer or a condition associated with an increased expression of GIT1 and MAT2B.

Embodiment 102A. The method of embodiment 101A, wherein the condition is hepatocellular carcinoma, colorectal cancer, or cholangiocarcinoma.

Embodiment 103A. The method of embodiment 101A, wherein the subject is a human.

Embodiment 104A. The method of embodiment 101A, wherein the inhibitor of GIT1 is administered topically, intravascularly, intravenously, intraarterially, intratumorally, intramuscularly, subcutaneously, intraperitoneally, intranasally, or orally.

Embodiment 10SA. The method of embodiment 101A, wherein the inhibitor of GIT1 is a compound of any one of embodiments 1A-90A.

Embodiment 106A. The method of embodiment 101A, further comprising administering an additional anti-cancer therapy.

Embodiment 107A. The method of embodiment 101A, wherein the subject in need thereof has or is at risk of having cancer.

Embodiment 108A. The method of embodiment 101A, wherein the subject in need thereof has or is undergoing chemotherapy and/or irradiation.

Embodiment 109A. The method of embodiment 101A, wherein the cancer comprises a hepatoblastoma, a cholangiocarcinoma, a colorectal cancer, carcinomas of the breast, lung cancer, oral squamous carcinoma, stomach cancer, or a cancer having an increased expression of GIT1 and MAT2B.

Embodiment 110A. The method of embodiment 110A, wherein the subject in need thereof has or is at risk of having a gastrointestinal disease.

Embodiment 111A. The method of embodiment 110A, wherein the gastrointestinal disease comprises of a disease in the liver or the colon.

Embodiment 112A. The method of embodiment 110A, wherein the gastrointestinal disease comprises of Crohn's Disease, ulcerative colitis, cirrhosis, hemochromatosis, Wilson's disease, autoimmune hepatitis, primary biliary cirrhosis, jaundice, diverticulitis, gastritis, chronic diarrhea, and necrotizing enterocolitis.

Embodiment 113A. The method of any one of embodiments 101A-112A, wherein the subject in need thereof is human.

Embodiment 114A. The method of embodiment 101A, wherein the inhibitor of GIT1 is:

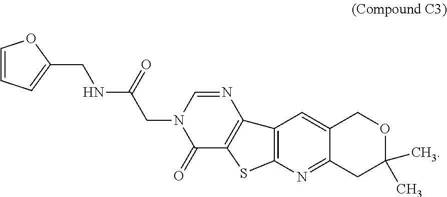 [SMILES: CC1(C)Cc2nc3sc4c(=O)n(CC(=O)NCc5ccco5)cnc4c3cc2CO1]

Embodiment 115A. A kit for treating, preventing, or reducing the likelihood of having, reducing the severity of and/or slowing the progression of a condition in a subject, comprising: an inhibitor of GIT1's normal interactome; and instructions for using the GIT1 inhibitor to treat, prevent, reduce the likelihood of having, reduce the severity of and/or slow the progression of the condition in the subject.

Embodiment 116A. The kit of embodiment 115A, wherein the GIT1 inhibitor is a compound of any one of embodiments 1A-90A.

Embodiment 117A. The kit of embodiment 115A, further comprising a device to administer the GIT1 inhibitor to a subject in need thereof.

Embodiment 118A. The kit of embodiment 115A, wherein the GIT1 inhibitor is:

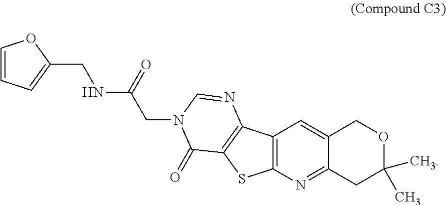 [SMILES: CC1(C)Cc2nc3sc4c(=O)n(CC(=O)NCc5ccco5)cnc4c3cc2CO1]

Embodiments include those listed below.

In various embodiments, the present invention provides a compound of Formula (A):

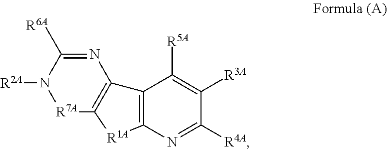

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{3A} is H, halo, optionally substituted alkyl, or OR^{9A}, where R^{9A} is H or optionally substituted alkyl;
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl; and
R^{7A} is C═O or CH₂; or
R^{3A} and R_{4A} can be linked together to form a ring, wherein the ring is optionally substituted; or
R^{3A} and R^{5A} can be linked together to form a ring, wherein the ring is optionally substituted.

In various embodiments, the present invention provides a compound of Formula (A):

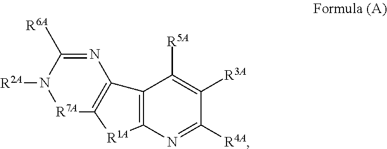

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{3A} is H, halo, optionally substituted alkyl, or OR^{9A}, where R^{9A} is H or optionally substituted alkyl;
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl; and
R^{7A} is C═O or CH₂; or
R^{3A} and R_{4A} can be linked together to form a ring, wherein the ring is optionally substituted; or
R^{3A} and R^{5A} can be linked together to form a ring, wherein the ring is optionally substituted, with the proviso that the compound of Formula (A) is not

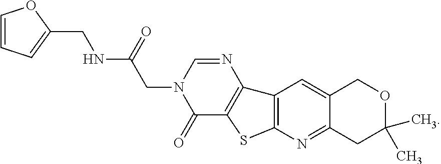 [SMILES: CC1(C)Cc2nc3sc4c(=O)n(CC(=O)NCc5ccco5)cnc4c3cc2CO1]

In various embodiments, the present invention provides a compound of Formula (A-1):

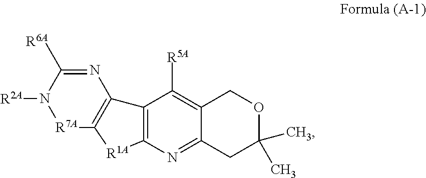

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{5A} is H, halo, or optionally substituted alkyl;
R^{6A} is H, halo, or optionally substituted alkyl; and
R^{7A} is C═O or CH₂.

In various embodiments, the present invention provides a compound of Formula (A-1):

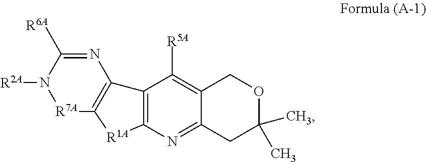

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{5A} is H, halo, or optionally substituted alkyl;
R^{6A} is H, halo, or optionally substituted alkyl; and
R^{1A} is C═O or CH₂, with the proviso that the compound of Formula (A-1) is not

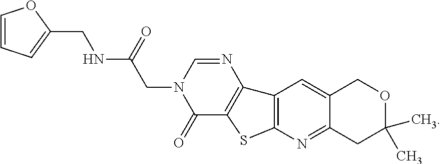 [SMILES: CC1(C)Cc2nc3sc4c(=O)n(CC(=O)NCc5ccco5)cnc4c3cc2CO1]

In various embodiments, the present invention provides a compound of Formula (A-2):

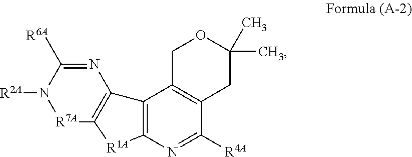

R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{4A} is H, halo, optionally substituted alkyl, or OR^{10A}, where R^{10A} is H or optionally substituted alkyl;
R^{6A} is H, halo, or optionally substituted alkyl; and
R^{7A} is C═O or CH₂.

In various embodiments, the present invention provides a compound of Formula (A-3):

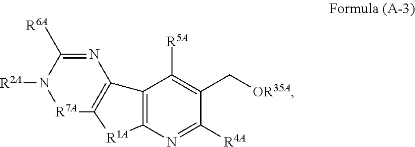

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl;
R^{7A} is C═O or CH₂; and
R^{35A} is H, CH₃, or CH₂CH₂OH.

In various embodiments, the present invention provides a compound of Formula (A-4):

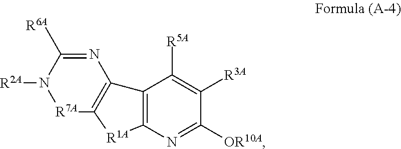

wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{3A} is H, halo, optionally substituted alkyl, or OR^{9A}, where R^{9A} is H or optionally substituted alkyl;
R^{6A} is H, halo, or optionally substituted alkyl;
R^{7A} is C═O or CH₂; and
R^{10A} is H, CH₃, or CH₂CH₂OH.

In some embodiments, R^{8A} is H, CH₃, or CH(CH₃)₂.

In some embodiments, R^{6A} is H, CH₃ or CH(CH₃)₂.

In some embodiments, R^{3A} is CH₂OR^{35A}, where R^{35A} is H, CH₃, or CH₂CH₂OH.

In some embodiments, R^{2A} is:

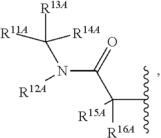

wherein:
R^{1A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl.

In some embodiments, R^{2A} is:

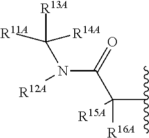

wherein:
R^{1A} is H, halo, optionally substituted alkyl, optionally substituted aryl, optionally substituted heteroaryl, optionally substituted cyclyl, optionally substituted heterocyclyl;
R^{12A} is H, CH₃, or CH(CH₃)₂;
R^{13A} is H, CH₃, or CH(CH₃)₂;
R^{14A} is H, CH₃, or CH(CH₃)₂;
R^{15A} is H, CH₃, or CH(CH₃)₂; and
R^{16A} is H, CH₃, or CH(CH₃)₂.

In some embodiments, R^{2A} is;

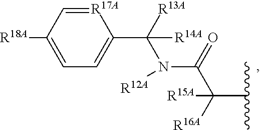

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{12A} is N or CH; and
R^{15A} is H or halo.

In some embodiments, R^{2A} is:

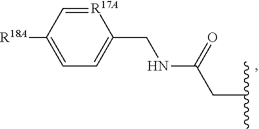

R^{12A} is N or CH; and
R^{18A} is H or F.

In some embodiments, R^{2A} is:

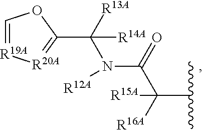

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl;
R^{19A} is N or CH; and
R^{20A} is N or CH.

In some embodiments, R^{2A} is:

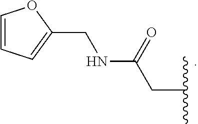 [SMILES: *CC(=O)NCc1ccco1]

In some embodiments, R^{2A} is:

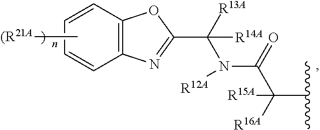

wherein:
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl;
R^{16A} is H, halo, or optionally substituted alkyl;
R^{21A} is independently H, halo, or optionally substituted alkyl; and
n is 1, 2 3, or 4.

In some embodiments, R^{2A} is:

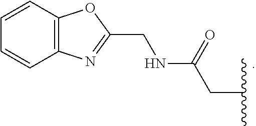 [SMILES: *CC(=O)NCc1nc2ccccc2o1]

In some embodiments, R^{2A} is:

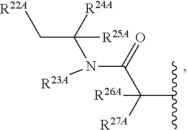

wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl.

In some embodiments, R^{2A} is;

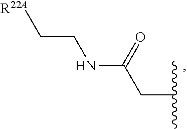

wherein:
R^{22A} is:

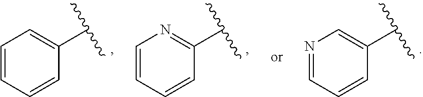 [SMILES: *c1ccccc1 ; *c1ccccn1 ; *c1cccnc1]

In some embodiments, R^{2A} is:

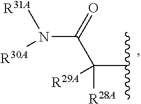

wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted.

In some embodiments, R^{2A} is:

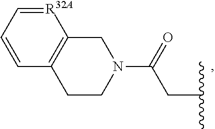

wherein: R^{32A} is CH or N.

In some embodiments, R^{2A} is:

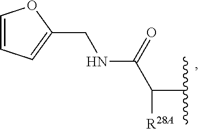

wherein: R^{28A} is H or CH₃; or

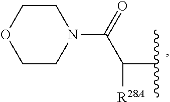

wherein: R^{28A} is H or CH₃;

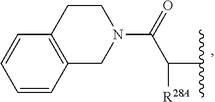

wherein: R^{28A} is H or CH₃; or

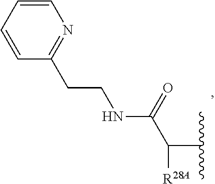

wherein: R^{28A} is H or CH₃.

In some embodiments, R^{2A} is:

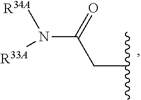

wherein,
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

In some embodiments, R^{2A} is:

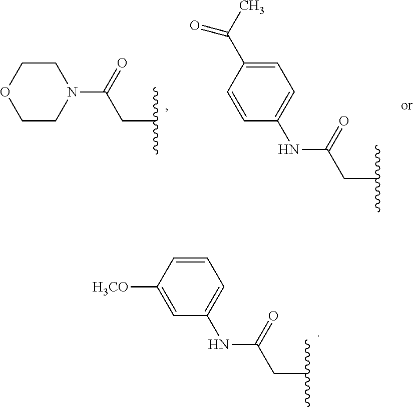 [SMILES: *CC(=O)N1CCOCC1 ; *CC(=O)Nc1ccc(C(C)=O)cc1 ; *CC(=O)Nc1cccc(OC)c1]

In some embodiments, R^{2A} is:

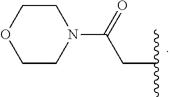 [SMILES: *CC(=O)N1CCOCC1]

In some embodiments, R^{2A} is:

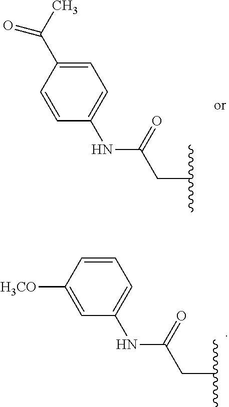 [SMILES: *CC(=O)Nc1ccc(C(C)=O)cc1 ; *CC(=O)Nc1cccc(OC)c1]

In some embodiments, R^{2A} is:

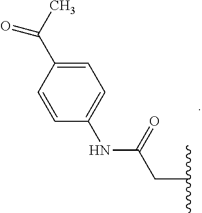 [SMILES: *CC(=O)Nc1ccc(C(C)=O)cc1]

In some embodiments, R^{2A} is:

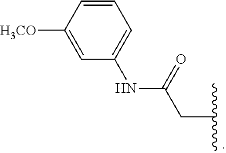 [SMILES: *CC(=O)Nc1cccc(OC)c1]

In some embodiments, the compound of Formula (A) is:

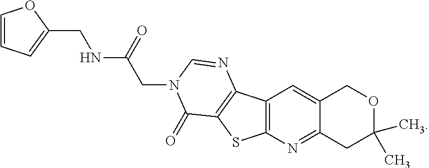 [SMILES: CC1(C)Cc2nc3sc4c(=O)n(CC(=O)NCc5ccco5)cnc4c3cc2CO1]

In some embodiments, the compound of Formula (A) is not:

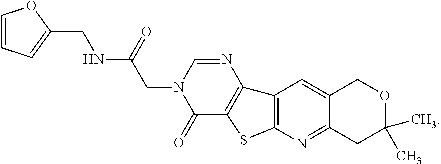 [SMILES: CC1(C)Cc2nc3sc4c(=O)n(CC(=O)NCc5ccco5)cnc4c3cc2CO1]

In some embodiments, the compound of Formula (A) is:

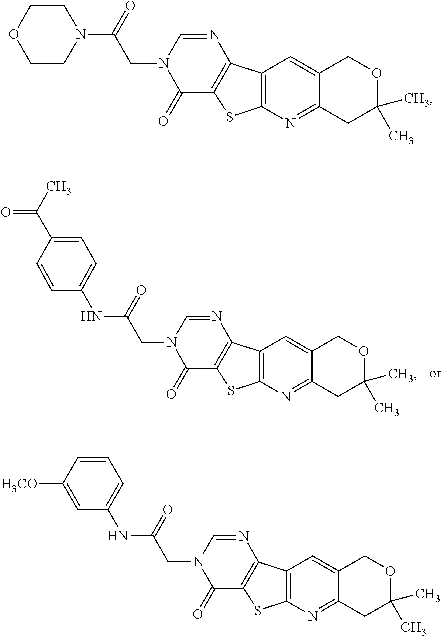 [SMILES: CC1(C)Cc2nc3sc4c(=O)n(CC(=O)N5CCOCC5)cnc4c3cc2CO1 ; CC(=O)c1ccc(NC(=O)Cn2cnc3c(sc4nc5c(cc43)COC(C)(C)C5)c2=O)cc1 ; COc1cccc(NC(=O)Cn2cnc3c(sc4nc5c(cc43)COC(C)(C)C5)c2=O)c1]

In some embodiments, the compound of Formula (A) is:

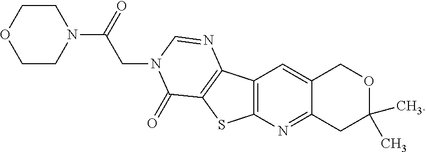 [SMILES: CC1(C)Cc2nc3sc4c(=O)n(CC(=O)N5CCOCC5)cnc4c3cc2CO1]

In some embodiments, the compound of Formula (A) is:

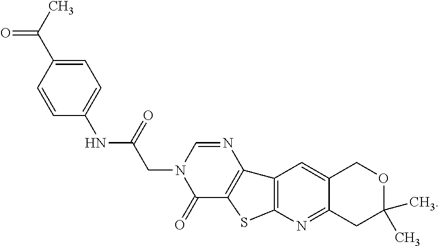 [SMILES: CC(=O)c1ccc(NC(=O)Cn2cnc3c(sc4nc5c(cc43)COC(C)(C)C5)c2=O)cc1]

In some embodiments, the compound of Formula (A) is:

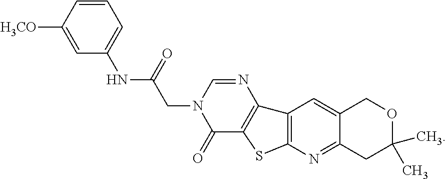 [SMILES: COc1cccc(NC(=O)Cn2cnc3c(sc4nc5c(cc43)COC(C)(C)C5)c2=O)c1]

In some embodiments, the compound of Formula (A-1) is:

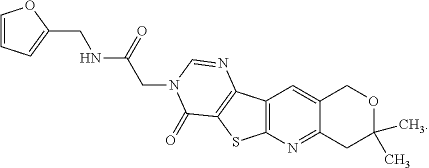 [SMILES: CC1(C)Cc2nc3sc4c(=O)n(CC(=O)NCc5ccco5)cnc4c3cc2CO1]

In some embodiments, the compound of Formula (A-1) is not:

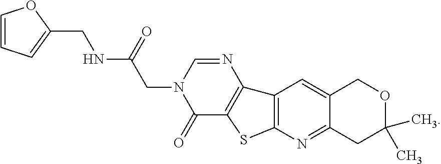 [SMILES: CC1(C)Cc2nc3sc4c(=O)n(CC(=O)NCc5ccco5)cnc4c3cc2CO1]

In some embodiments, the compound of Formula (A-1) is:

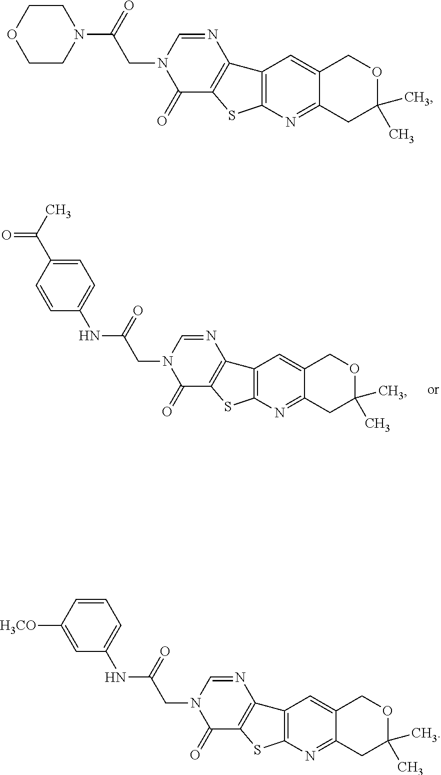 [SMILES: CC1(C)Cc2nc3sc4c(=O)n(CC(=O)N5CCOCC5)cnc4c3cc2CO1 ; CC(=O)c1ccc(NC(=O)Cn2cnc3c(sc4nc5c(cc43)COC(C)(C)C5)c2=O)cc1 ; COc1cccc(NC(=O)Cn2cnc3c(sc4nc5c(cc43)COC(C)(C)C5)c2=O)c1]

In some embodiments, the compound of Formula (A-1) is:

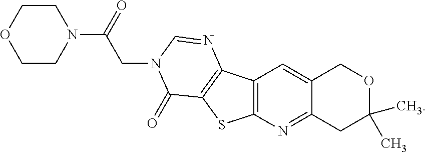 [SMILES: CC1(C)Cc2nc3sc4c(=O)n(CC(=O)N5CCOCC5)cnc4c3cc2CO1]

In some embodiments, the compound of Formula (A-1) is:

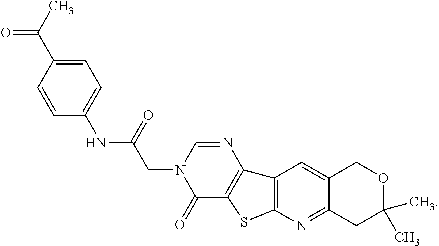 [SMILES: CC(=O)c1ccc(NC(=O)Cn2cnc3c(sc4nc5c(cc43)COC(C)(C)C5)c2=O)cc1]

In some embodiments, the compound of Formula (A-1) is:

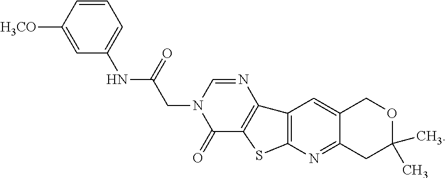 [SMILES: COc1cccc(NC(=O)Cn2cnc3c(sc4nc5c(cc43)COC(C)(C)C5)c2=O)c1]

In various embodiments, the present invention provides a compound of Formula (I), Formula (II), Formula (III), Formula (IV), Formula (V), Formula (VI), Formula (VII), Formula (VIII), Formula (IX), Formula (X), or Formula (XI). In some embodiments, the present invention provides a compound of Formula (I). In some embodiments, the present invention provides a compound of Formula (II). In some embodiments, the present invention provides a compound of Formula (III). In some embodiments, the present invention provides a compound of Formula (IV). In some embodiments, the present invention provides a compound of Formula (V). In some embodiments, the present invention provides a compound of Formula (VI). In some embodiments, the present invention provides a compound of Formula (VII). In some embodiments, the present invention provides a compound of Formula (VIII). In some embodiments, the present invention provides a compound of Formula (IX). In some embodiments, the present invention provides a compound of Formula (X). In some embodiments, the present invention provides a compound of Formula (XI).

In various embodiments, the present invention provides a compound of Formula (A), Formula (A-1), Formula (A-2), Formula (A-3), or Formula (A-4). In some embodiments, the compound of Formula (A-1) is a compound of Formula (A). In some embodiments, the compound of Formula (A-2) is a compound of Formula (A). In some embodiments, the compound of Formula (A-3) is a compound of Formula (A). In some embodiments, the compound of Formula (A-4) is a compound of Formula (A). In some embodiments, the present invention provides a compound of Formula (A). In some embodiments, the present invention provides a compound of Formula (A-1). In some embodiments, the present invention provides a compound of Formula (A-2). In some embodiments, the present invention provides a compound of Formula (A-3). In some embodiments, the present invention provides a compound of Formula (A-4).

In various embodiments, the compounds of the present invention inhibit GIT1. In various embodiments, the compounds of the present invention interrupt or inhibit GI1T-MAT2B interaction. In various embodiments, the compounds of the present invention interrupt or inhibit GIT1 from interacting with MAT2B and RAF/MEK/ERK. In various embodiments, the compounds of the present invention interrupt or inhibit GIT1 from interacting with MAT2B and RAF/MEK/ERK to halt cancer cell growth.

Embodiments include those listed below.

In various embodiments, the present invention provides a composition comprising a compound of the present invention. In some embodiments, the composition is a pharmaceutical composition.

In various embodiments, the present invention provides a composition, wherein the composition comprises a compound of Formula (I), Formula (II), Formula (III), Formula (IV), Formula (V), Formula (VI), Formula (VII), Formula (VIII), Formula (IX), Formula (X), or Formula (XI), or any combination thereof. In some embodiments, the composition is a pharmaceutical composition.

In various embodiments, the present invention provides a composition, wherein the composition comprises a compound of Formula (A), Formula (A-1), Formula (A-2), Formula (A-3), or Formula (A-4), or any combination thereof. In some embodiments, the composition is a pharmaceutical composition.

Embodiments include those listed below.

In various embodiments, the present invention provides a GIT1 inhibitor. In various embodiments, the GIT1 inhibitor is a compound of the present invention. In some embodiments the GIT1 inhibitor is a compound of Formula (I), Formula (II), Formula (III), Formula (IV), Formula (V), Formula (VI), Formula (VII), Formula (VIII), Formula (IX), Formula (X), or Formula (XI), or a combination thereof. In some embodiments, the GIT1 inhibitor is a compound of Formula (I). In some embodiments, the GIT1 inhibitor is a compound of Formula (II). In some embodiments, the GIT1 inhibitor is a compound of Formula (III). In some embodiments, the GIT1 inhibitor is a compound of Formula (IV). In some embodiments, the GIT1 inhibitor is a compound of Formula (V). In some embodiments, the GIT1 inhibitor is a compound of Formula (VI). In some embodiments, the GIT1 inhibitor is a compound of Formula (VII). In some embodiments, the GIT1 inhibitor is a compound of Formula (VIII). In some embodiments, the GIT1 inhibitor is a compound of Formula (IX). In some embodiments, the GIT1 inhibitor is a compound of Formula (X). In some embodiments, the GIT1 inhibitor is a compound of Formula (XI). In some embodiments, the GIT1 inhibitor is a compound of Formula (A), Formula (A-1), Formula (A-2), Formula (A-3), or Formula (A-4), or any combination thereof. In some embodiments, the GIT1 inhibitor is a compound of Formula (A). In some embodiments, the GIT1 inhibitor is a compound of Formula (A-1). In some embodiments, the GIT1 inhibitor is a compound of Formula (A-2). In some embodiments, the GIT1 inhibitor is a compound of Formula (A-3). In some embodiments, the GIT1 inhibitor is a compound of Formula (A-4).

Embodiments include those listed below.

In various embodiments, the present invention provides a pharmaceutical composition comprising a GIT1 inhibitor. In some embodiments the GIT1 inhibitor is a compound of Formula (I), Formula (II), Formula (III), Formula (IV), Formula (V), Formula (VI), Formula (VII), Formula (VIII), Formula (IX), Formula (X), or Formula (XI), or a combination thereof. In some embodiments, the GIT1 inhibitor is a compound of Formula (I). In some embodiments, the GIT1 inhibitor is a compound of Formula (II). In some embodiments, the GIT1 inhibitor is a compound of Formula (III). In some embodiments, the GIT1 inhibitor is a compound of Formula (IV). In some embodiments, the GIT1 inhibitor is a compound of Formula (V). In some embodiments, the GIT1 inhibitor is a compound of Formula (VI). In some embodiments, the GIT1 inhibitor is a compound of Formula (VII). In some embodiments, the GIT1 inhibitor is a compound of Formula (VIII). In some embodiments, the GIT1 inhibitor is a compound of Formula (IX). In some embodiments, the GIT1 inhibitor is a compound of Formula (X). In some embodiments, the GIT1 inhibitor is a compound of Formula (XI).

In some embodiments, the GIT1 inhibitor is a compound of Formula (A), Formula (A-1), Formula (A-2), Formula (A-3), or Formula (A-4), or any combination thereof. In some embodiments, the GIT1 inhibitor is a compound of Formula (A). In some embodiments, the GIT1 inhibitor is a compound of Formula (A-1). In some embodiments, the GIT1 inhibitor is a compound of Formula (A-2). In some embodiments, the GIT1 inhibitor is a compound of Formula (A-3). In some embodiments, the GIT1 inhibitor is a compound of Formula (A-4).

Embodiments include those listed below.

In various embodiments, the present invention provides a method of treating, preventing, reducing the likelihood of having, reducing the severity of and/or slowing the progression of a condition in a subject comprising: administering a therapeutically effective amount of an inhibitor of GIT1's normal interactome to the subject, thereby treating, preventing, reducing the likelihood of having, reducing the severity of and/or slowing the progression of the condition in the subject, wherein the condition is cancer or a condition associated with an increased expression of GIT1 and MAT2B.

In various embodiments, the present invention provides a method of treating a condition in a subject comprising: administering a therapeutically effective amount of an inhibitor of GIT1's normal interactome to the subject, thereby treating the condition in the subject, wherein the condition is cancer or a condition associated with an increased expression of GIT1 and MAT2B.

In various embodiments, the present invention provides a method of preventing a condition in a subject comprising: administering a therapeutically effective amount of an inhibitor of GIT1's normal interactome to the subject, thereby preventing the condition in the subject, wherein the condition is cancer or a condition associated with an increased expression of GIT1 and MAT2B.

In various embodiments, the present invention provides a method of reducing the likelihood of having a condition in a subject comprising: administering a therapeutically effective amount of an inhibitor of GIT1's normal interactome to the subject, thereby reducing the likelihood of having the condition in the subject, wherein the condition is cancer or a condition associated with an increased expression of GIT1 and MAT2B.

In some embodiments, the condition is cancer. In some embodiments, the condition is hepatocellular carcinoma, colorectal cancer, or cholangiocarcinoma. In some embodiments, the condition is hepatocellular carcinoma. In some embodiments, the condition is colorectal cancer. In some embodiments, the condition is cholangiocarcinoma. In some embodiments, the cancer comprises a hepatoblastoma, a cholangiocarcinoma, a colorectal cancer, carcinomas of the breast, lung cancer, oral squamous carcinoma, stomach cancer, or a cancer having an increased expression of GIT1 and MAT2B. In some embodiments, the condition is gastrointestinal disease.

Embodiments include those listed below.

In various embodiments, the present invention provides a method of treating, preventing, reducing the likelihood of having, reducing the severity of and/or slowing the progression of a condition in a subject comprising: administering a therapeutically effective amount of an inhibitor of GIT1 inhibitor to the subject, thereby treating, preventing, reducing the likelihood of having, reducing the severity of and/or slowing the progression of the condition in the subject.

In various embodiments, the present invention provides a method of treating a condition in a subject comprising: administering a therapeutically effective amount of a GIT1 inhibitor to the subject, thereby treating the condition in the subject.

In various embodiments, the present invention provides a method of preventing a condition in a subject comprising: administering a therapeutically effective amount of a GIT1 inhibitor to the subject, thereby preventing the condition in the subject.

In various embodiments, the present invention provides a method of reducing the likelihood of having a condition in a subject comprising: administering a therapeutically effective amount of a GIT1 inhibitor to the subject, thereby reducing the likelihood of having the condition in the subject.

In various embodiments, the present invention provides a method of reducing the severity of a condition in a subject comprising: administering a therapeutically effective amount of a GIT1 inhibitor to the subject, thereby reducing the severity of the condition in the subject.

In various embodiments, the present invention provides a method of slowing the progression of a condition in a subject comprising: administering a therapeutically effective amount of a GIT1 inhibitor to the subject, thereby slowing the progression of the condition in the subject.

In some embodiments, the condition is cancer. In some embodiments, the condition is hepatocellular carcinoma, colorectal cancer, or cholangiocarcinoma. In some embodiments, the condition is hepatocellular carcinoma. In some embodiments, the condition is colorectal cancer. In some embodiments, the condition is cholangiocarcinoma. In some embodiments, the cancer comprises a hepatoblastoma, a cholangiocarcinoma, a colorectal cancer, carcinomas of the breast, lung cancer, oral squamous carcinoma, stomach cancer, or a cancer having an increased expression of GIT1 and MAT2B. In some embodiments, the condition is gastrointestinal disease.

Embodiments include those listed below.

In various embodiments, the present invention provides a method of treating, preventing, reducing the likelihood of having, reducing the severity of and/or slowing the progression of a condition in a subject comprising: administering a therapeutically effective amount of a compound of the present invention to the subject, thereby treating, preventing, reducing the likelihood of having, reducing the severity of and/or slowing the progression of the condition in the subject.

In various embodiments, the present invention provides a method of treating a condition in a subject comprising: administering a therapeutically effective amount of a compound to the subject, thereby treating the condition in the subject, wherein the compound is a compound of Formula (A), Formula (A-1), Formula (A-2), Formula (A-3), Formula (A-4), or combination thereof.

In various embodiments, the present invention provides a method of preventing a condition in a subject comprising: administering a therapeutically effective amount of a compound to the subject, thereby preventing the condition in the subject, wherein the compound is a compound of Formula (A), Formula (A-1), Formula (A-2), Formula (A-3), Formula (A-4), or combination thereof.

In various embodiments, the present invention provides a method of reducing the likelihood of having a condition in a subject comprising: administering a therapeutically effective amount of compound to the subject, thereby reducing the likelihood of having the condition in the subject, wherein the compound is a compound of Formula (A), Formula (A-1), Formula (A-2), Formula (A-3), Formula (A-4), or combination thereof.

In various embodiments, the present invention provides a method of reducing the severity of a condition in a subject comprising: administering a therapeutically effective amount of compound to the subject, thereby reducing the severity of the condition in the subject, wherein the compound is a compound of Formula (A), Formula (A-1), Formula (A-2), Formula (A-3), Formula (A-4), or combination thereof.

In various embodiments, the present invention provides a method of slowing the progression of a condition in a subject comprising: administering a therapeutically effective amount of compound to the subject, thereby slowing the progression of the condition in the subject, wherein the compound is a compound of Formula (A), Formula (A-1), Formula (A-2), Formula (A-3), Formula (A-4), or combination thereof.

In some embodiments, the condition is cancer. In some embodiments, the condition is hepatocellular carcinoma, colorectal cancer, or cholangiocarcinoma. In some embodiments, the condition is hepatocellular carcinoma. In some embodiments, the condition is colorectal cancer.

In some embodiments, the condition is cholangiocarcinoma. In some embodiments, the cancer comprises a hepatoblastoma, a cholangiocarcinoma, a colorectal cancer, carcinomas of the breast, lung cancer, oral squamous carcinoma, stomach cancer, or a cancer having an increased expression of GIT1 and MAT2B. In some embodiments, the condition is gastrointestinal disease.

Embodiments include those listed below.

In various embodiments, the present invention provides a method of treating, preventing, reducing the likelihood of having, reducing the severity of and/or slowing the progression of a condition in a subject comprising: administering a therapeutically effective amount of a composition to the subject, thereby treating, preventing, reducing the likelihood of having, reducing the severity of and/or slowing the progression of the condition in the subject, wherein the composition comprises a compound of the present invention.

In various embodiments, the present invention provides a method of treating a condition in a subject comprising: administering a therapeutically effective amount of a composition to the subject, thereby treating the condition in the subject, wherein the composition comprises a compound of Formula (A), Formula (A-1), Formula (A-2), Formula (A-3), Formula (A-4), or combination thereof.

In various embodiments, the present invention provides a method of preventing a condition in a subject comprising: administering a therapeutically effective amount of a composition to the subject, thereby preventing the condition in the subject, wherein the composition comprises a compound of Formula (A), Formula (A-1), Formula (A-2), Formula (A-3), Formula (A-4), or combination thereof.

In various embodiments, the present invention provides a method of reducing the likelihood of having a condition in a subject comprising: administering a therapeutically effective amount of a composition to the subject, thereby reducing the likelihood of having the condition in the subject, wherein the composition comprises a compound of Formula (A), Formula (A-1), Formula (A-2), Formula (A-3), Formula (A-4), or combination thereof.

In various embodiments, the present invention provides a method of reducing the severity of a condition in a subject comprising: administering a therapeutically effective amount of a composition to the subject, thereby reducing the severity of the condition in the subject, wherein the composition comprises a compound of Formula (A), Formula (A-1), Formula (A-2), Formula (A-3), Formula (A-4), or combination thereof.

In various embodiments, the present invention provides a method of slowing the progression of a condition in a subject comprising: administering a therapeutically effective amount of a composition to the subject, thereby slowing the progression of the condition in the subject, wherein the composition comprises a compound of Formula (A), Formula (A-1), Formula (A-2), Formula (A-3), Formula (A-4), or combination thereof.

In some embodiments, the condition is cancer. In some embodiments, the condition is hepatocellular carcinoma, colorectal cancer, or cholangiocarcinoma. In some embodiments, the condition is hepatocellular carcinoma. In some embodiments, the condition is colorectal cancer. In some embodiments, the condition is cholangiocarcinoma. In some embodiments, the cancer comprises a hepatoblastoma, a cholangiocarcinoma, a colorectal cancer, carcinomas of the breast, lung cancer, oral squamous carcinoma, stomach cancer, or a cancer having an increased expression of GIT1 and MAT2B. In some embodiments, the condition is gastrointestinal disease.

Embodiments include those listed below.

In various embodiments, the present invention provides a kit for treating, preventing, or reducing the likelihood of having, reducing the severity of and/or slowing the progression of a condition in a subject, comprising: a GIT1 inhibitor of its normal interactome; and instructions for using the GIT1 inhibitor to treat, prevent, reduce the likelihood of having, reduce the severity of and/or slow the progression of the condition in the subject.

In various embodiments, the present invention provides a kit for treating, preventing, or reducing the likelihood of having, reducing the severity of and/or slowing the progression of a condition in a subject, comprising: a GIT1 inhibitor; and instructions for using the GIT1 inhibitor to treat, prevent, reduce the likelihood of having, reduce the severity of and/or slow the progression of the condition in the subject.

In various embodiments, the present invention provides a kit for treating, preventing, or reducing the likelihood of having, reducing the severity of and/or slowing the progression of a condition in a subject, comprising: a composition to treat, prevent, reduce the likelihood of having, reduce the severity of and/or slow the progression of the condition in the subject; and instructions for using the composition to treat, prevent, reduce the likelihood of having, reduce the severity of and/or slow the progression of the condition in the subject.

In various embodiments, the present invention provides a kit for treating, preventing, or reducing the likelihood of having, reducing the severity of and/or slowing the progression of a condition in a subject, comprising: a compound to treat, prevent, reduce the likelihood of having, reduce the severity of and/or slow the progression of the condition in the subject; and instructions for using the compound to treat, prevent, reduce the likelihood of having, reduce the severity of and/or slow the progression of the condition in the subject.

Embodiments include those listed below.

In various embodiments, the present invention provides a use of a compound of the present invention. In various embodiments, the present invention provides a use of a compound of the present invention as a medicament.

In various embodiments, the present invention provides a use of a composition of the present invention. In various embodiments, the present invention provides a use of a composition of the present invention as a medicament.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Compound C3 was PubChem Compound ID #663383 (2-(8,8-Dimethyl-4-oxo-7,10-dihydro-8H-pyrano[3″,4″: 5′,6′]pyrido[3′,2′: 4,5]thieno[3,2-d]pyrimidin-3 (4H)-yl)-N-(2-furylmethyl) acetamide).

### Example 1

Determining Domain at which MAT2B Binds to GIT1.

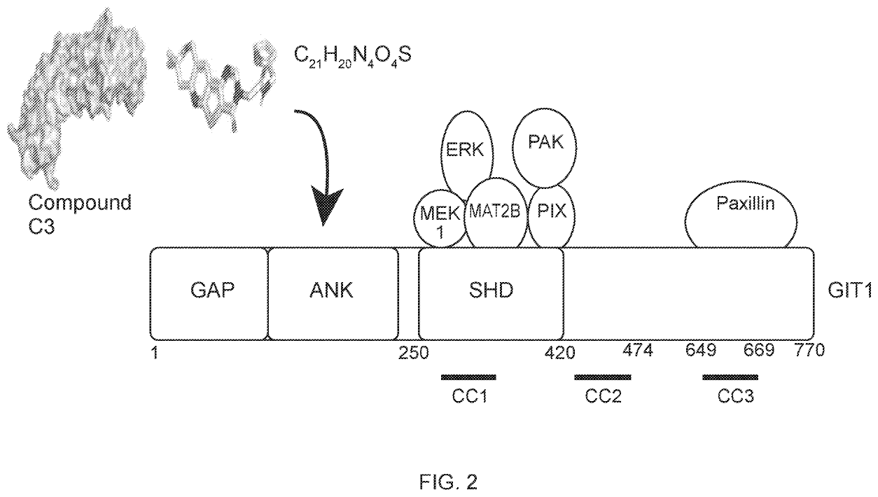

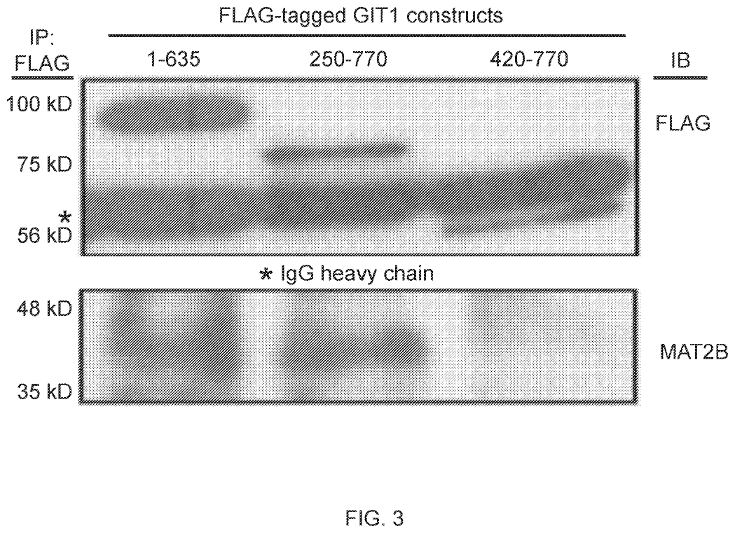

In various embodiments, the present invention provides cancer treatments by using a compound to inhibit GIT1 interaction with MAT2B. A general scheme is shown in FIG. 2. FIG. 3 reveals that FLAG-GIT1 (1-635aa) and FLAG-GIT1 (250-770aa) pulled down endogenous MAT2B successfully, but FLAG-GIT1 (420-770aa) did not. This suggests that 250-420aa of GIT1, which corresponds to the SHD domain, is where MAT2B binds. The inventors discovered the SHD domain is inaccessible, so the ANK domain was chosen as the target for small inhibitory molecules instead.

### Example 2

Treatment of colorectal cancers (CRC) and hepatocellular carcinoma (HCC) by novel compounds and methods that inhibit GIT1 interaction with MAT2B to reduce activation of RAS-RAF-MEK-ERK signaling that enhances tumor growth and invasion.

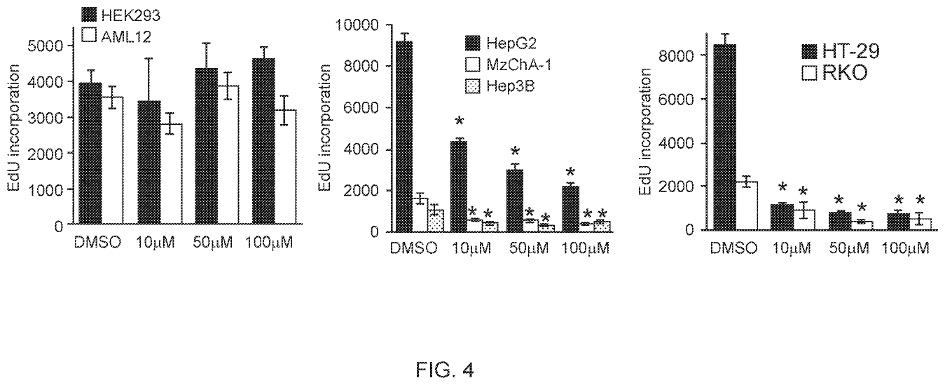

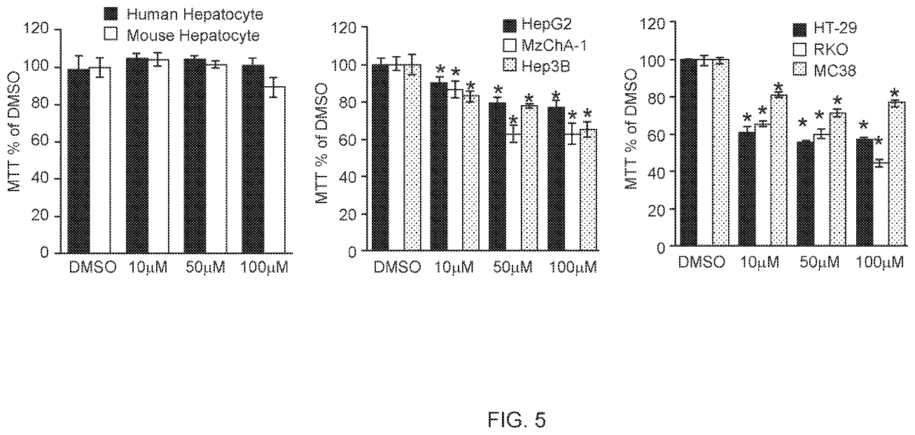

The inventors identified nine inhibitors and perfomed an EdU assay after treating non-malignant (HEK293, AML12) and malignant (HepG2, MzChA-1, HT-29, RKO) cells with varying concentrations of GIT1 inhibitor for 24 hours (results are mean±SE from 3 independent experiments done in duplicates, *p<0.05 vs. DMSO). An MTT assay was also performed after treating primary human and mouse hepatocytes and liver and colon cancer cells with varying concentrations of GIT1 inhibitor for 24 hours (results are mean±SE from 3 independent experiments done in duplicates, *p<0.05 vs. DMSO). The results are shown in FIGS. 4 and 5. Compound C3 exhibited the most potent growth inhibitory effect and was cytotoxic in cancer cells at 10 μM but not in non-malignant cells. Percoll may be used to remove dead human hepatocytes.

### Example 3

### In Vitro and In Vivo Studies on Exemplary GIT1 Inhibitors

Compound C3 was dissolved in pure DMSO at 100 mM as stock, then dissolved in DMSO to 10 mM when used. This solution was added to culture dishes at a final concentration of 1-10 μM as needed for experiments.

### Effect of Compound C3 on Cell Cycle Progression

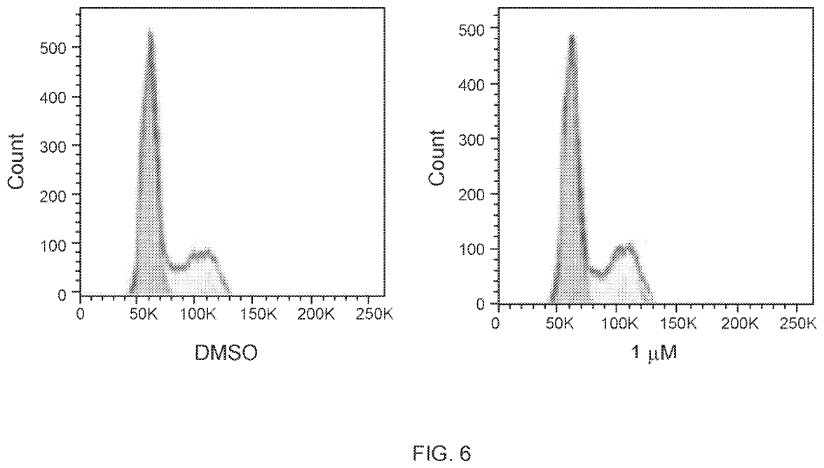

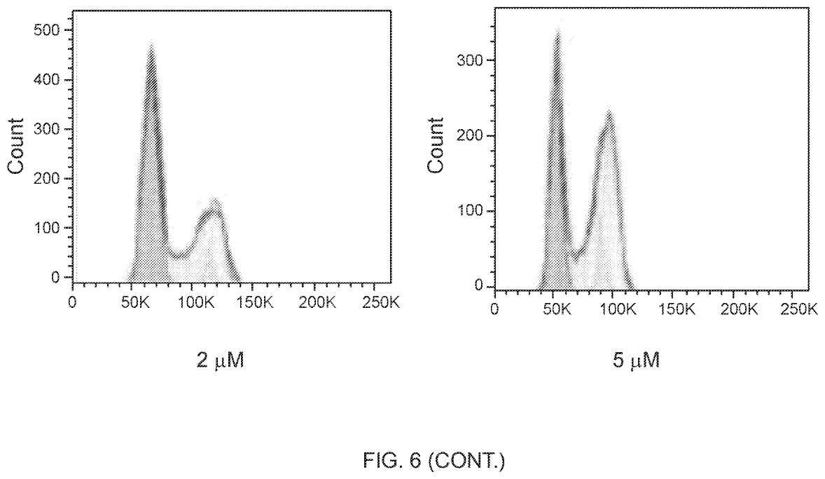

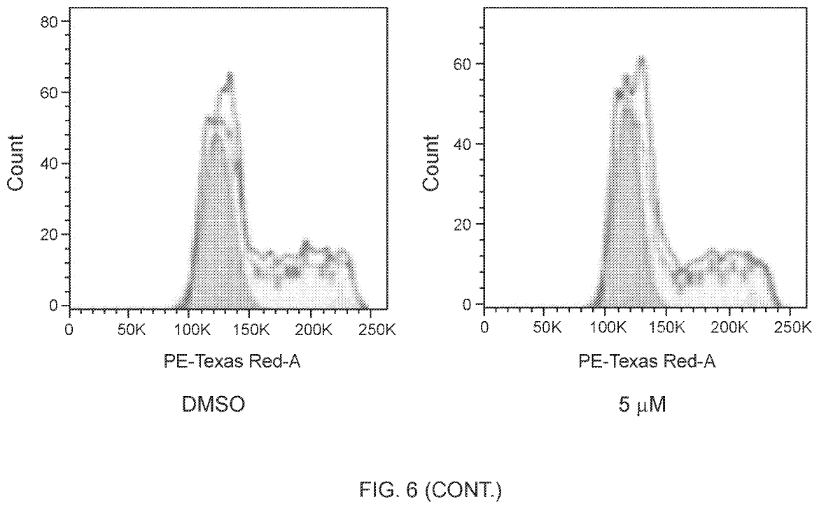

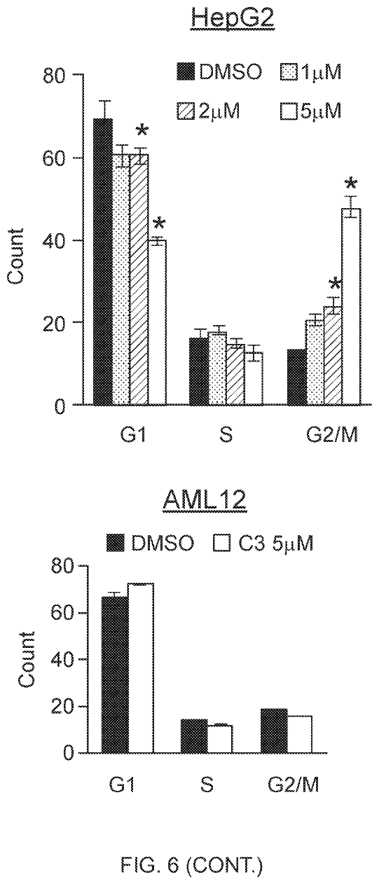

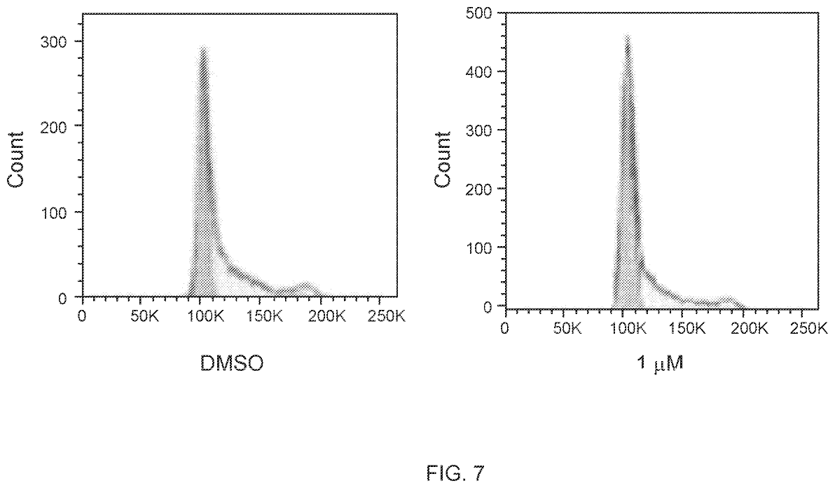

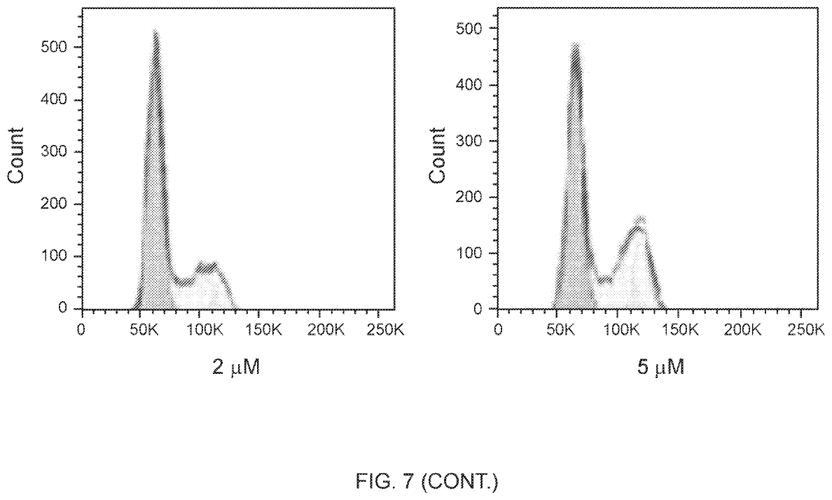

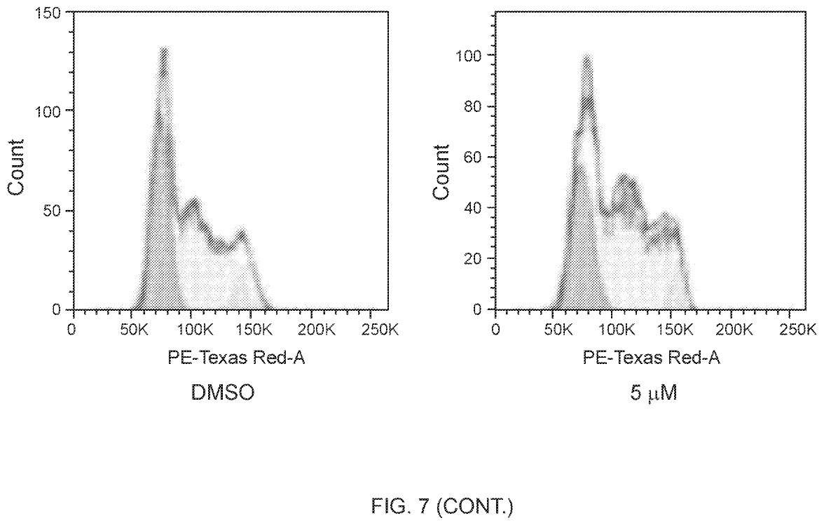

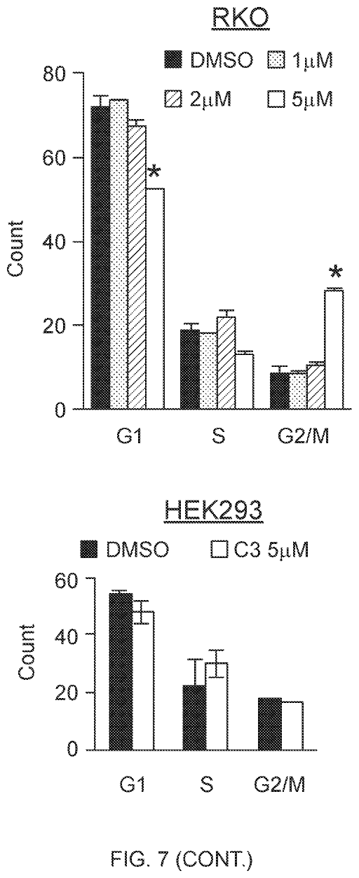

FIG. 6 shows results of a binding assay with flow cytometry for HEPG2 and AML12 cells synchronized by serum starvation for 48 hours and released into serum containing media and treated with 1-5 μM of a GIT1 inhibitor (compound C3) or DMSO control for 24 hours prior to FACS (results are from representative experiments, done independently at least three times; *p<0.05 vs. DMSO). FIG. 7 shows results of a binding assay with flow cytometry for RKO and HEK293 cells synchronized by serum starvation for 48 hours and released into serum containing media and treated with 1-5 μM of a GIT1 inhibitor or DMSO for 24 hours prior to FACS (results are from representative experiments, done independently at least three times; *p<0.05 vs. DMSO). In HEPG2 and RKO cells, treatment with compound C3 lowered the percentage of cells in G1 phase, while G2/M phase was significantly increased, suggesting G2/M phase arrest in cancer cells. This did not occur with AML12 or HEK293 cells.

### Effect of Compound C3 on Cell Apoptosis

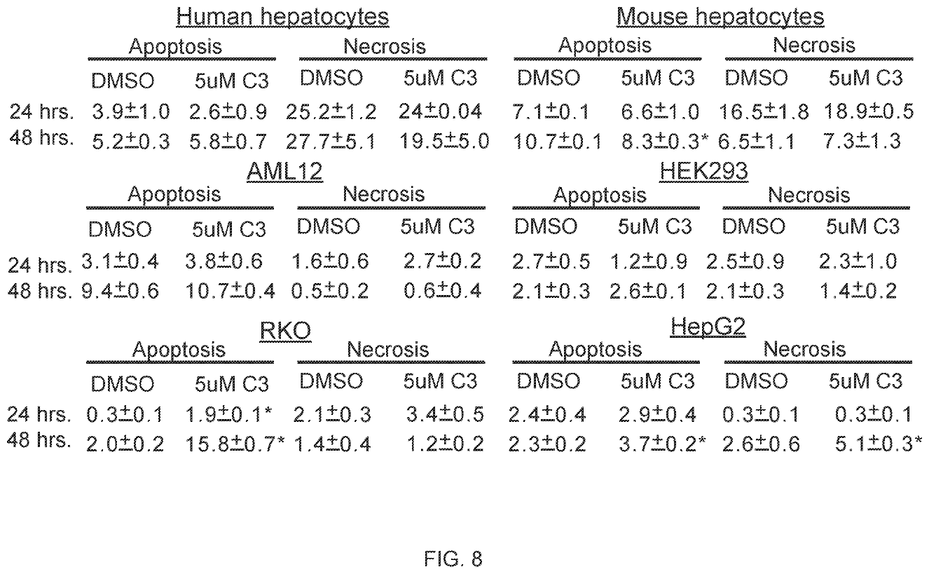

An annexin V-FITC/propidium iodide (PI) binding assay was performed with flow cytometry. The results are shown in FIG. 8. As seen from the data in FIG. 8, 5 μM compound C3 induces significant apoptosis in RKO and HepG2 cells, and significant necrosis in HepG2 cells. Compound C3 at 5 μM does not induce cell death in human or mouse hepatocytes, AML12, or HEK293 cells. Both G2/M arrest and cell death are mechanisms by which compound C3 inhibits cancer cells' growth.

### Effect of Compound C3 on Clonogenic Potential and Migration of Cancer Cells.

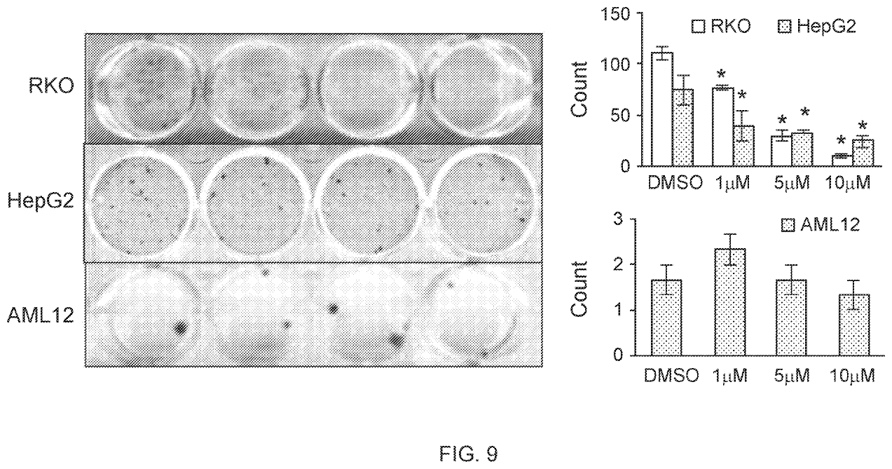

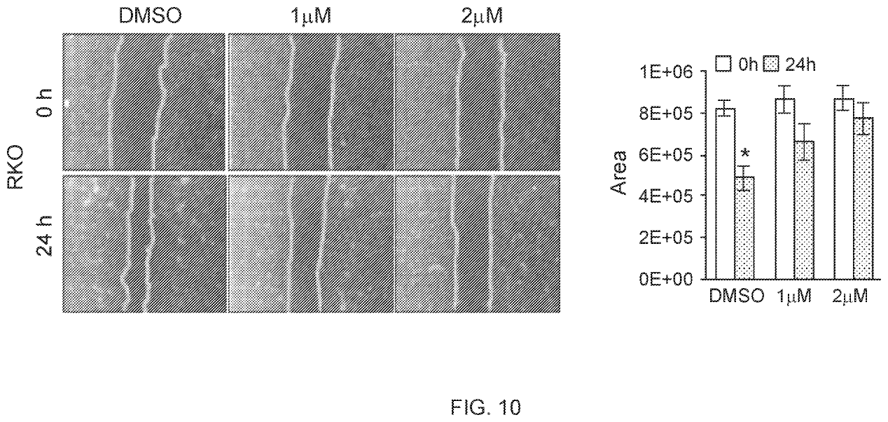

RKO, HepG2, and AML12 cells were treated with 1-10 μM compound C3 for 24 hours and maintained in drug free medium for 10-20 days. Results of a migration assay for these cells is shown in FIG. 9 (results are from representative experiments done at least three times. *p<0.05 vs. DMSO), Compared to DMSO, compound C3 inhibited the clonogenic potential of both cancer cell types at 1 μM and the effect was dose dependent but had no effect on clonogenic potential of AML12 cells. FIG. 10 shows results of a migration assay of RKO cells treated with 1-2 μM compound C3 for 24 hours (results are from representative experiments done at least three times. *p<0.05 vs. DMSO). As can be seen by the results, compound C3 exerted a significant inhibition in migration of RKO cells at 1 μM.

### Effect of Compound C3 on MAT2B/GIT1/RAF/MEK/ERK Interactions and MEK and ERK Activity

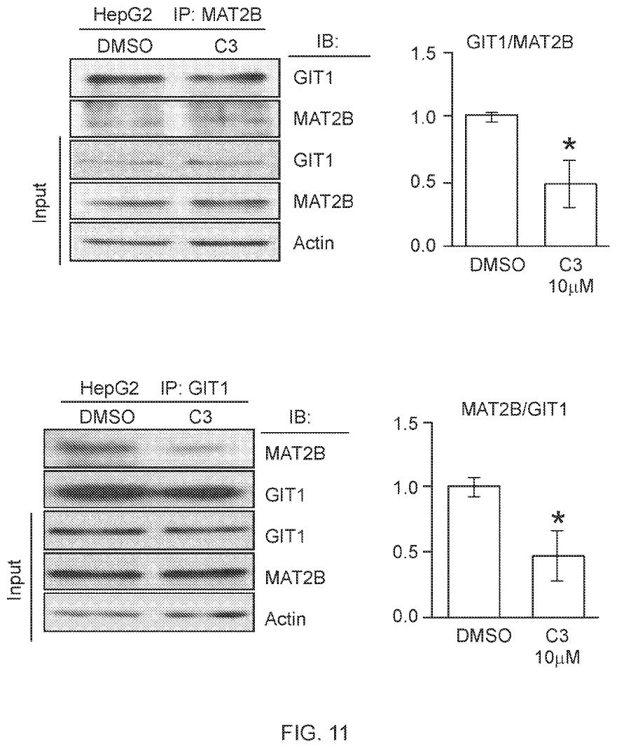

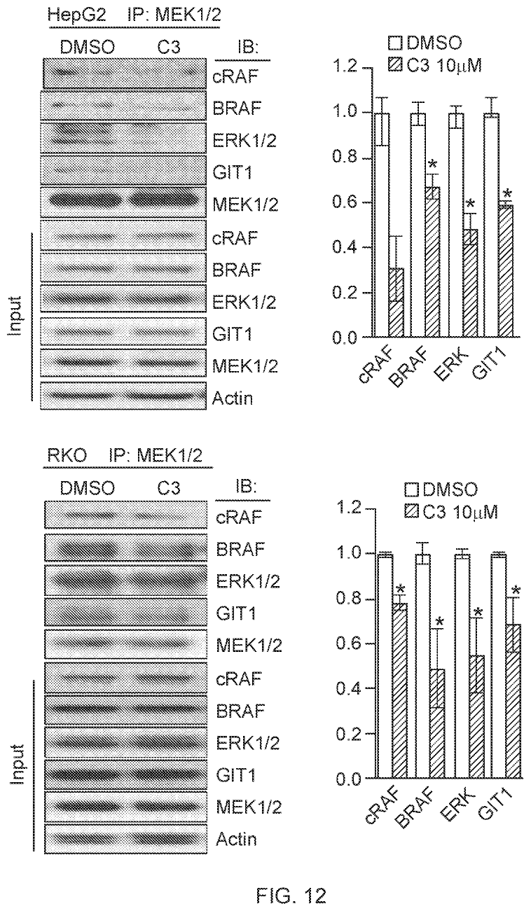

GIT1-MAT2B scaffold complex sequesters and activates RAS/RAF/MEK/ERK signaling. FIG. 11 shows the blots and densitometric changes from treating HepG2 cells with 10 μM of a GIT1 inhibitor for 24 hours and co-IP with anti-MAT2B (left) or anti-GIT1 (right) antibodies followed by immunoblotting (results are from at least 3 independent experiments in mean #SE. *p<0.05 vs. DMSO), FIG. 12 shows blots and densitometric changes from treating HepG2 (left) and RKO (right) cells with 10 μM of a GIT1 inhibitor for 24 hours followed by co-IP with anti-MEK1/2 antibody followed by immunoblotting for cRAF, BRAF, ERK1/2 and GIT1 (results are from at least 3 independent experiments in mean #SE. *p<0.05 vs. DMSO). The data shows that compound C3 disrupted GIT1-MAT2B scaffold formation and reduced RAF/MEK/ERK recruitment.

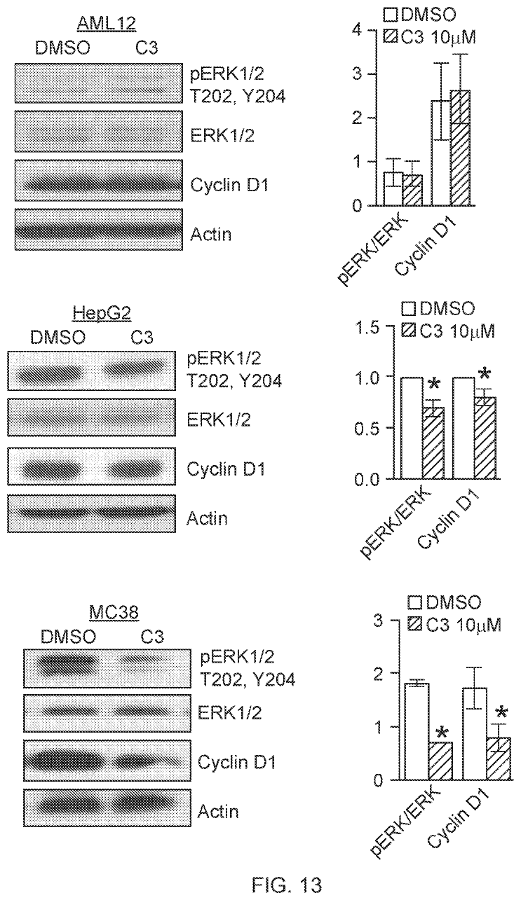

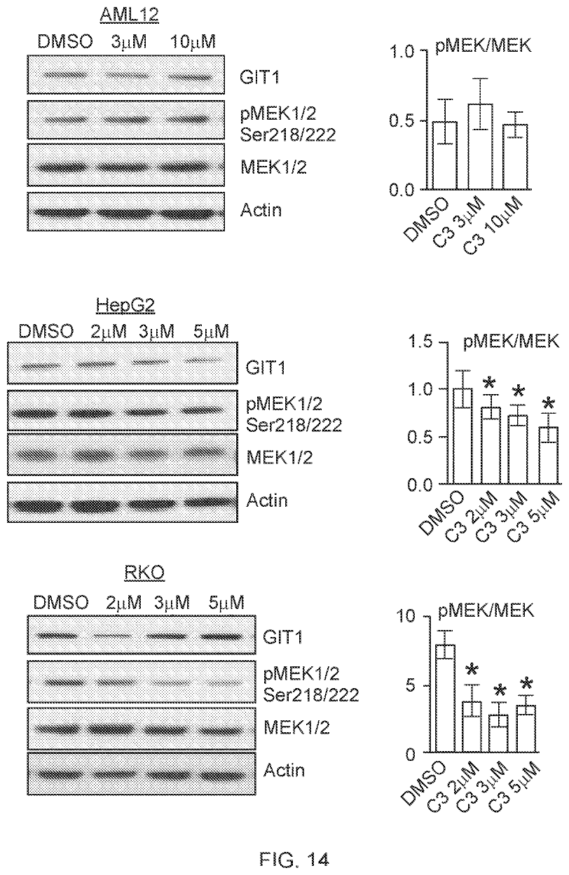

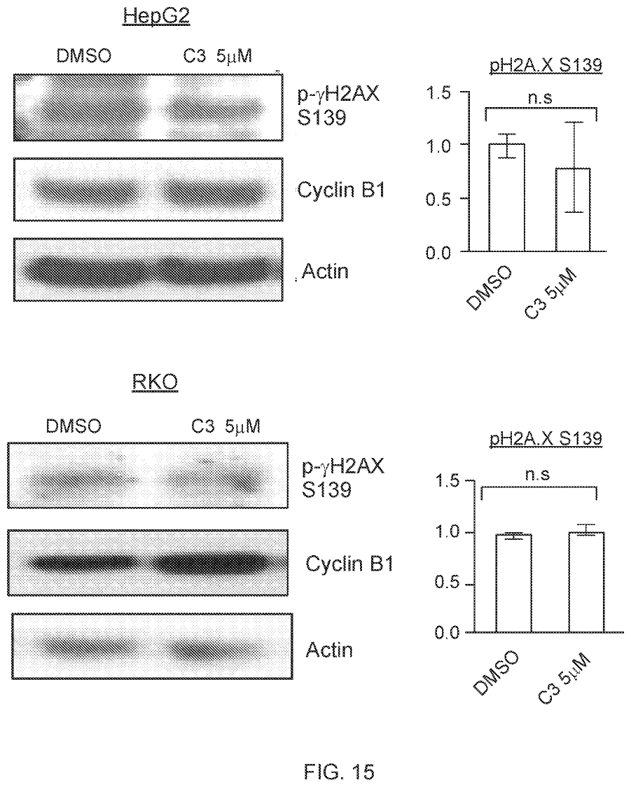

FIG. 13 shows the blots and densitometrie changes from treating AML12, HepG2, and MC38 cells with 10 μM of a GIT1 inhibitor for 24 hours followed by western blotting (results are from at least 3 independent experiments in mean±SE. *p<0.05 vs. DMSO). FIG. 14 shows the blots and densitometric changes from treating AML12, HepG2, and RKO cells with 2-10 μM of a GIT1 inhibitor for 24 hours followed by western blotting (results are from at least 3 independent experiments in mean±SE. *p<0.05 vs. DMSO). The data reveals ERK and MEK activities were lower in cancer cells HepG2, MC38, and RKO, but not in AML12 cells. Cyclin D1 is a downstream target of this signaling and consistently its protein level was lower in cancer cells but not in AML12 cells. Treatment of human hepatocytes with 10 μM compound C3 had no effect on MEK or ERK activity.

### Effect of Compound C3 on Cyclin B1 Expression

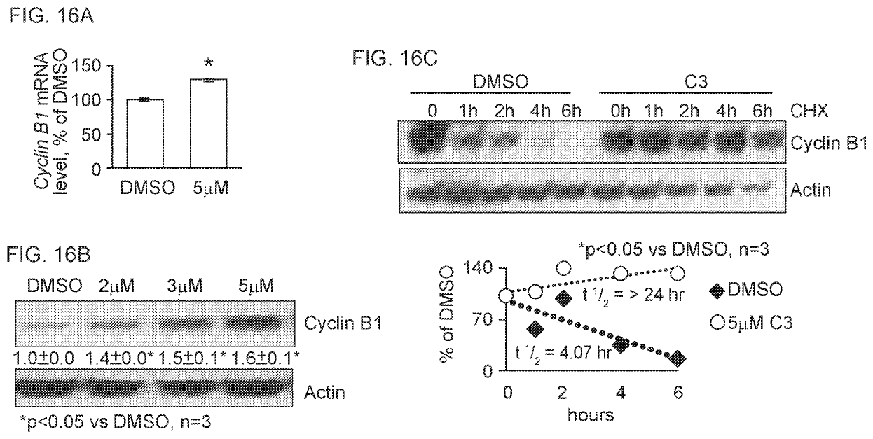

HepG2 cells were treated with DMSO or compound C3 (2-5 μM) for 24 hours. FIG. 16A shows cyclin BI expression by RT-PCR and (FIG. 16B) Western Blotting. Numbers shown below are mean±SE densitometric values. FIG. 16C shows results for cells that were pretreated with cycloheximide (CHX) and then with DMSO or a GIT1 inhibitor (5 μM) in time course (results are from at least 3 independent experiments in mean±SE. *p<0.05 vs. DMSO).

### Effect of Compound C3 on Cyclin B1/CDK1 Interaction and CDK1 Activation

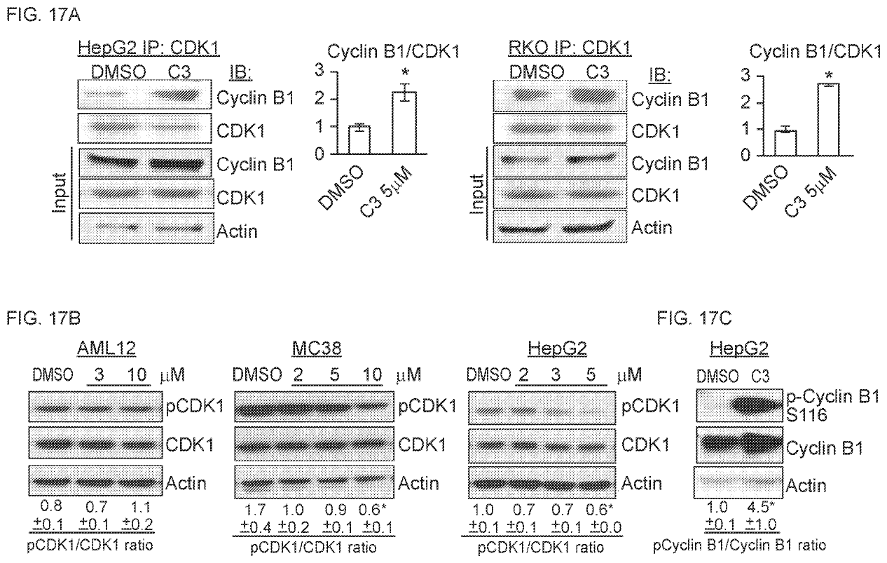

HepG2 and RKO cells were treated with 5 μM of compound C3 for 24 hours, followed by co-IP with anti-CDK1 antibody and western blots. Results can be seen in (FIG. 17A) and (FIG. 17B) shows results of treating AML12, MC38, and HepG2 cells with 2-10 μM of a GIT1 inhibitor for 24 hours and western blotting for pCDK1 (Thr14/Tyr15), total CDK1 and Actin. FIG. 17C shows results of treating HepG2 cells with a GIT1 inhibitor for 24 hours and western blotting for p-cyclin B1 (S116). The densitometric changes are from at least 3 independent experiments in mean±SE; *p<0.05 vs. DMSO. Binding of cyclin BI to CDK1 initiates conformational changes allowing CDK1 to alter its phosphorylation status and to become an active kinase.

### Effect of Compound C3 on GIT1-Cyclin B1 Interaction and Cyclin B1 Expression

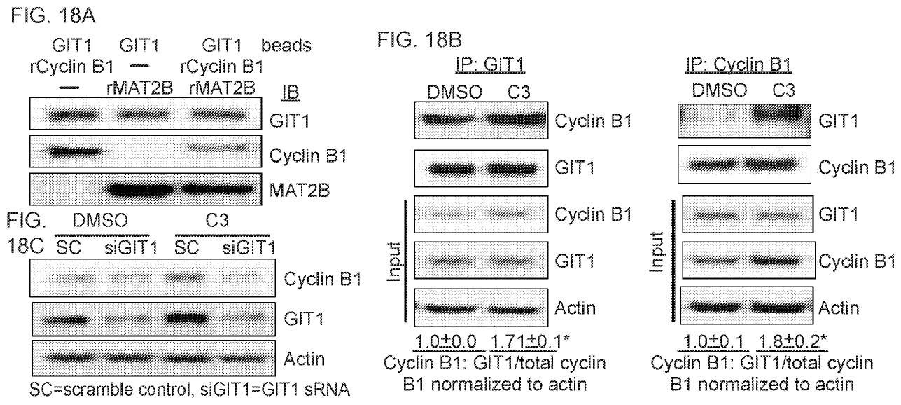

In vitro translated GIT1, recombinant MAT2B, and cyclin B1 (both 0.5 μg) were used to evaluate their direct binding, as shown in the blots of FIG. 18A. Also shown, is the results of (FIG. 18B) treating HepG2 cells with a GIT1 inhibitor at 10 μM for 24 hours and subjected to co-IP with anti-GIT1 or cyclin B1 antibody and immunoblotted for cyclin BI and GIT1. In vitro translated GIT1, recombinant MAT2B, and cyclin B1 (both 0.5 μg) were used to evaluate their direct binding. The ratio of cyclin BI bound to GIT1 was compared to DMSO control. FIG. 18C shows that compound C3-mediated increase in cyclin BI level requires GIT1 (*p<0.05 vs. DMSO from n=3). Taken together, treatment with compound C3 activated cyclin B1/CDK1, led to cyclin B1 accumulation by a mechanism that requires GIT1.

### Effect of Compound C3 on CRC Growth in a Syngeneic Subcutaneous Mouse Model

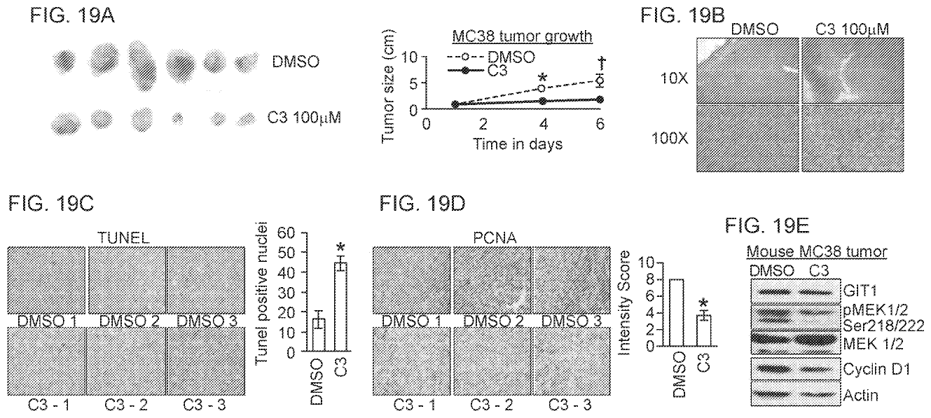

MC38 cells were injected subcutaneously in both flanks. Compound C3 and DMSO were injected intratumorally every other day at 100 μM (5×IC50) starting at day 7 when the tumors averaged 0.5 cm in diameter. Tumor size was measured by caliper, with the tumor volume calculated according to the formula: (π/6)*(length*width*height). Each mouse received compound C3 injection in tumors on one flank and DMSO in the contralateral tumor. Animals were sacrificed on day 13. FIG. 19A shows gross tumor appearance at day 13 and growth curve; (FIG. 19B) representative H&E staining of tumors; (FIG. 19C) TUNEL staining and score; (FIG. 19D) PCNA staining and score; and (FIG. 19E) western blotting of representative tumors (*p<0.05, †p<0.01 vs. DMSO). Body weights were not significantly different and H&E staining of major organs showed no tissue damage or toxicity at the dose given intratumorally. On H&E, the spindle MC38 tumor cells turned into small round cells upon compound C3 treatment and TUNEL assay showed more apoptopic cells. PCNA staning showed compound C3 had a dramatic effect in reducing its staining in the tumors. Western blots showed decreased MEK1/2 activity nad less cyclin D1 level in compound C3 treated tumors, indicating effectiveness of compound C3 in inhibiting MEK signaling.

### Effect of Compound C3 on Human CRC Growth in Liver

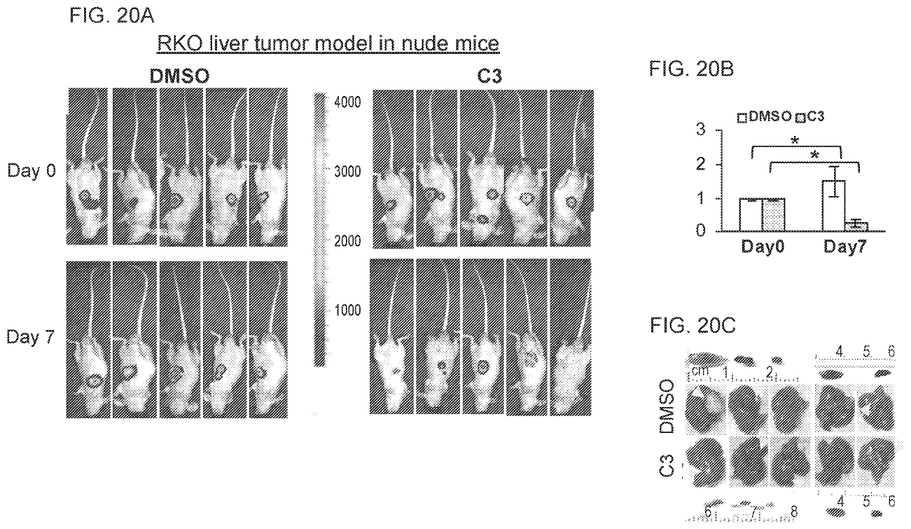

Compound C3 powder was dissolved in pure DMSO at 50 mg/ml, as a stock store in-80 °C. Stock solution was dissolved in 200 μl 0.9% saline to reach 25 mg/kg. For control group, DMSO was dissolved in 200 μl saline to achieve a concentration of DMSO in saline of 7.5%. RKO cells (2×106) stably expressing luciferase were injected directly into the liver of nude mice. Twenty days later DMSO and Compound C3 were injected intraperitoneally (ip) at a dose of 25 mg/kg daily for 5 days and imaged on day 7. FIG. 20A shows small animal imaging using IVIS Spectrum photon-counting device optical imaging system (Xenogen, Alameda, CA) at day 0 and 7 after start of treatment to track tumor progression; (FIG. 20B) a graph summarizing luciferase intensity; and (FIG. 20C) gross pictures of the livers at day 7, with arrows pointing to tumors (*p<0.05). Compound C3 was effective in reducing tumor growth in this model without causing overt toxicity.

### Effect of Compound C3 on CRC Liver Metastasis in Immune Competent Mice

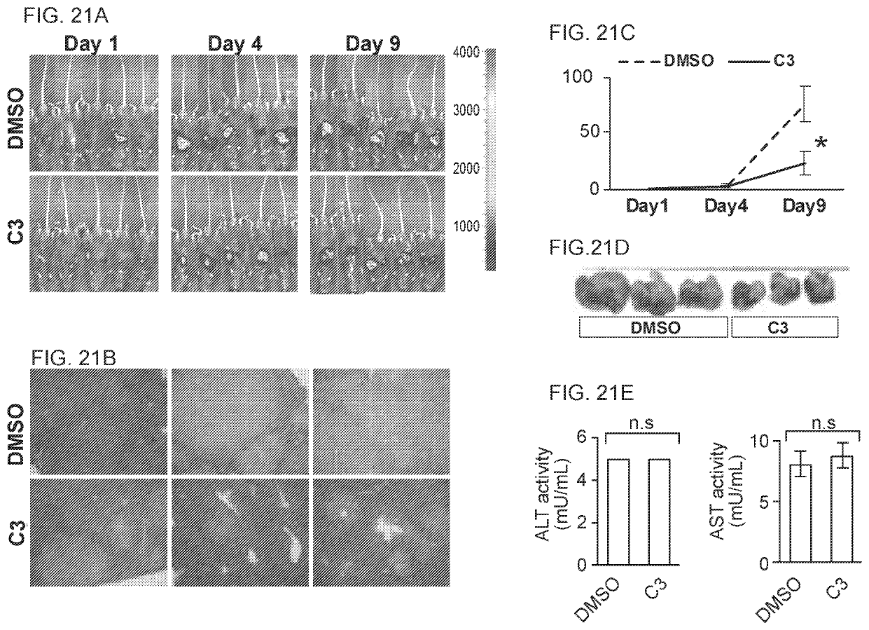

Compound C3 powder was dissolved in pure DMSO at 50 mg/ml, as a stock store in-80 °C. Stock solution was dissolved in 200 μl 0.9% saline to reach 15 mg/kg. For control group, DMSO was dissolved in 200 μl saline to achieve a concentration of DMSO in saline of 4.5%. MC38 cells (2.5×105) stably expressing luciferase were injected intrasplenically followed by splenectomy (CRLM model). Seven days later, Compound C3 (20 mg/kg on day 1, then 15 mg/kg daily ×3 days, rest for 2 days, then 15 mg/kg×2 days) or DMSO were administered ip. FIG. 21A shows small animal imaging after treatment started; (FIG. 21B) H&E of tumors; (FIG. 21C) luciferase intensity at various time points, (FIG. 21D) gross pictures of the livers at day 9; and (FIG. 21E) ALT and AST levels (*p<0.05 vs. DMSO). Tumor growth was inhibited without toxicity.

### Effect of Compound C3 on M2 Macrophages in the Tumor Microenvironment

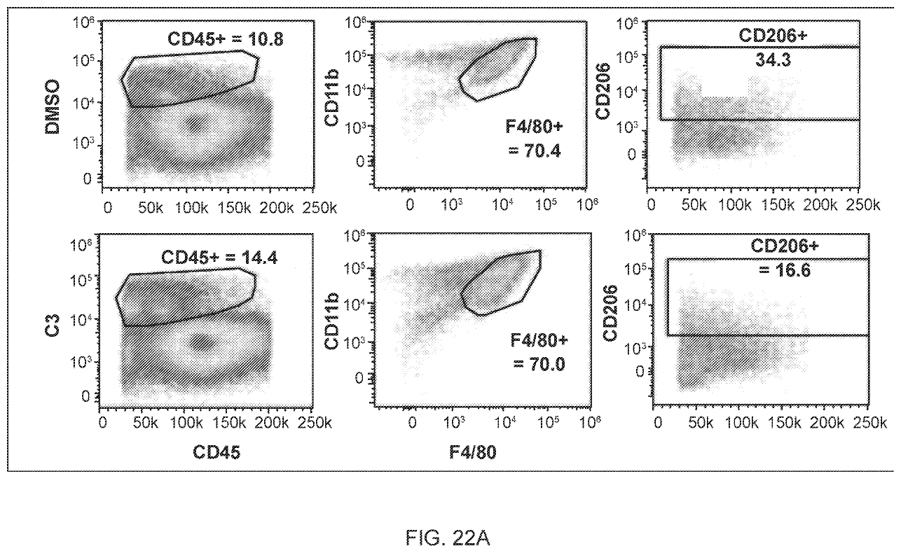

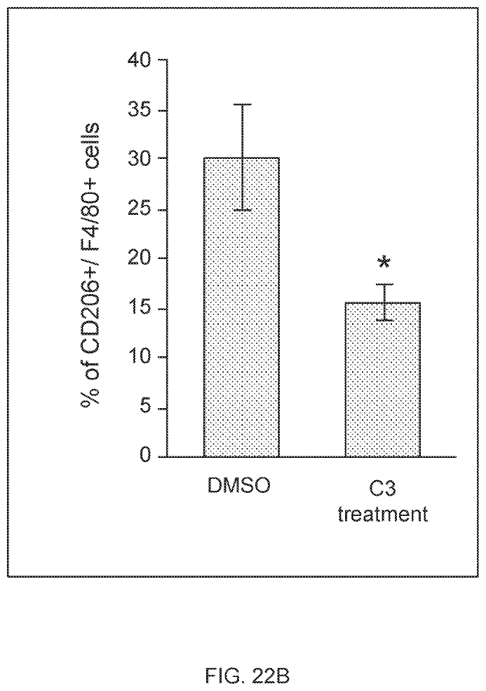

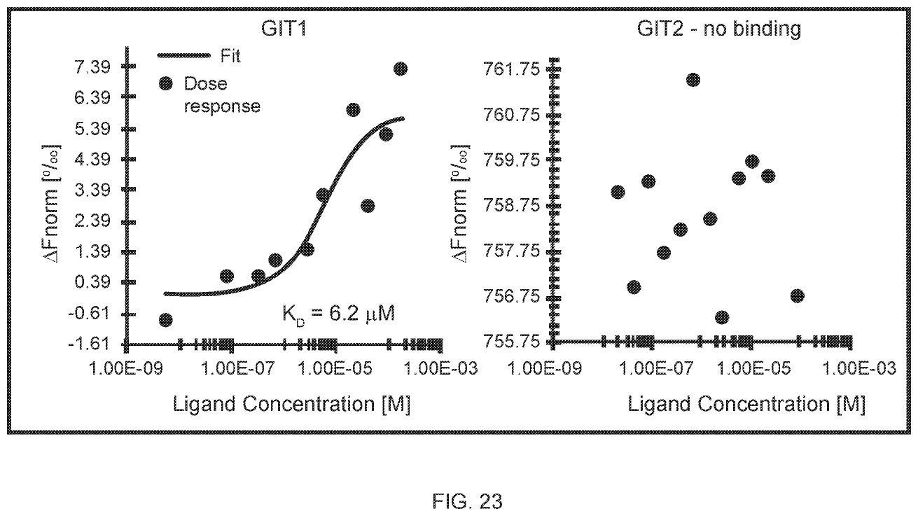

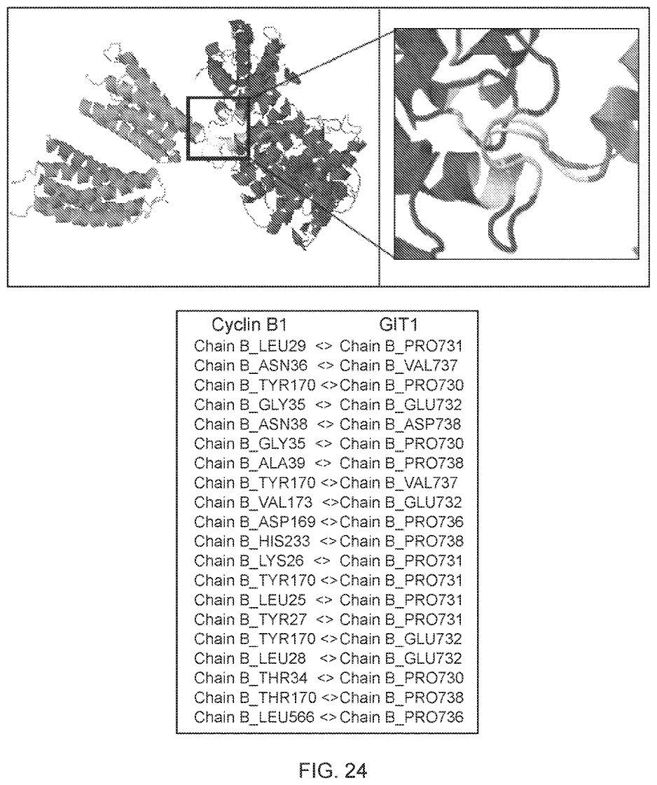

Liver tumors from CRLM model in syngeneic mice were processed for flow cytometry. FIG. 22A shows results of gating CD45+ for leucocytes; and then gating F4/80+CD11b+ for macrophages. In this subgroup, CD206+ was used to identify M2 macrophages. FIG. 22B shows CD206+ macrophages population in CRLM tumor samples treated with DMSO or Compound C3 as in the bar graph (3-month-old male mice, n=6 each group) (*p<0.05 vs. DMSO). Compound C3 dramatically reduced M2 macrophages in the tumors. There was also strong indication that compound C3 raised MI and CD8+ T cells.

### Effect of Compound C3 on Growth of CRC Organoid

An EdU assay of CRC organoids from AKPS mice (APCF/Fp53F/FKRAS+SMAD4KOCDX2ERT+) was treated with 10M of compound C3 for 24 hours. Low and high magnifications, as seen in FIG. 26, show compound C3 does not affect appearance. Compound C3 inhibits CRC organoids growth.

### Example 4

Effect of GIT1 Mutation on Interaction with Cyclin B1

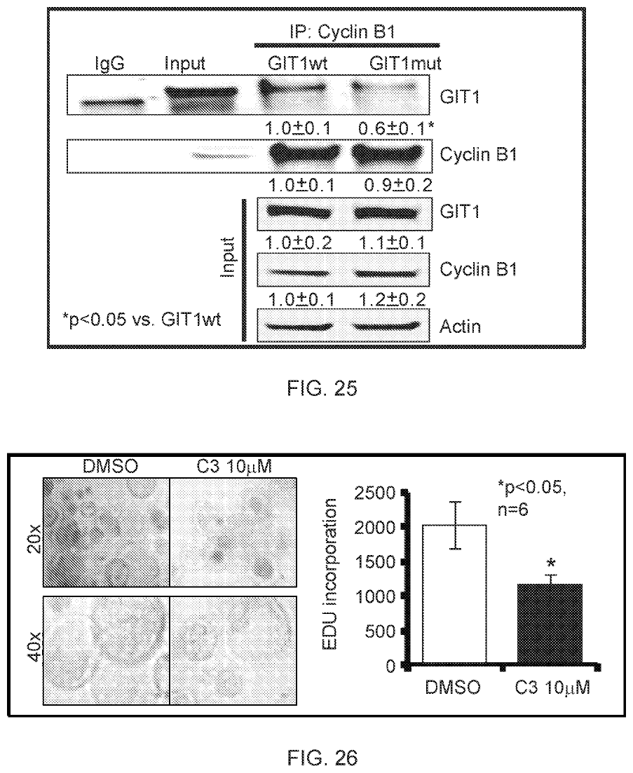

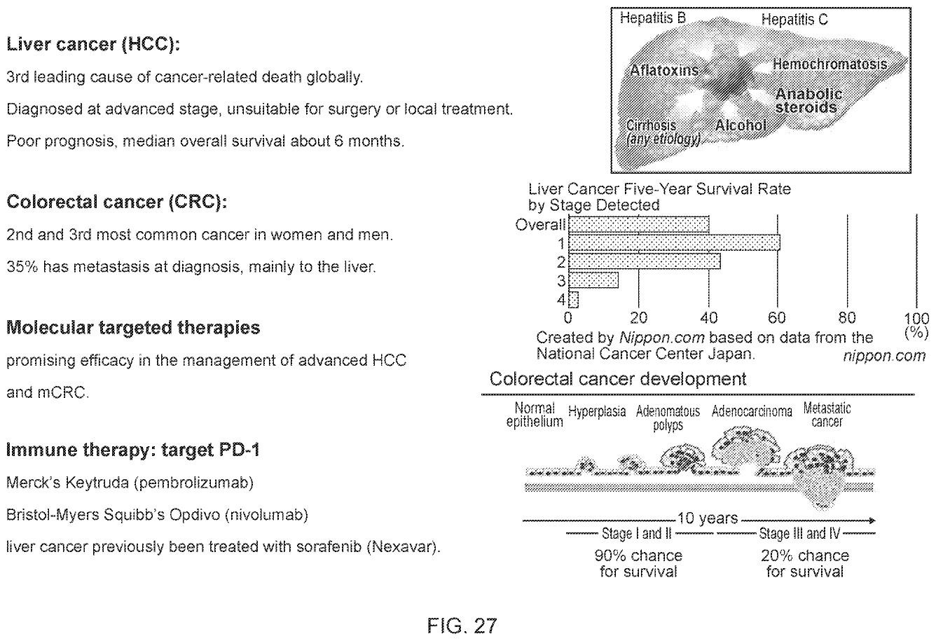

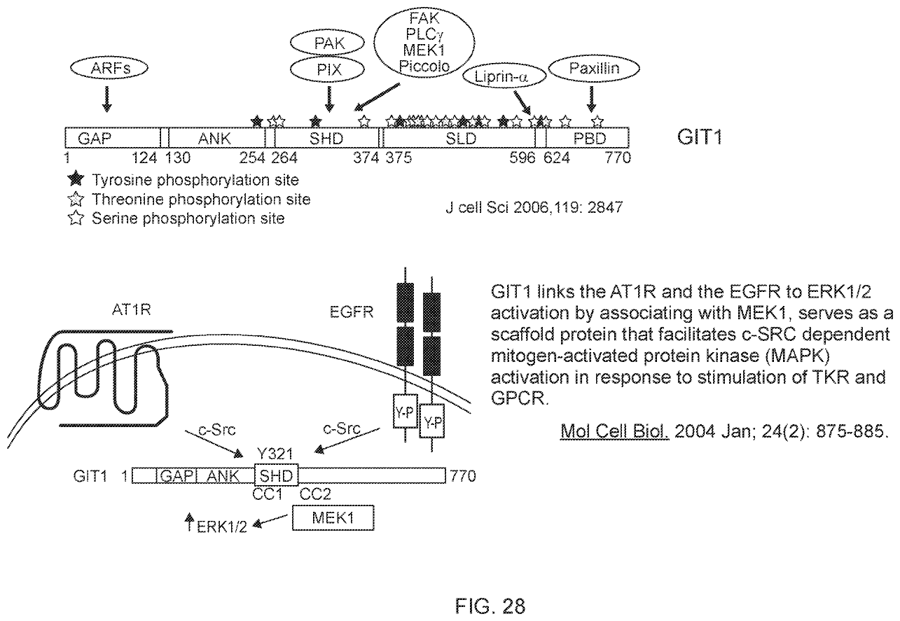

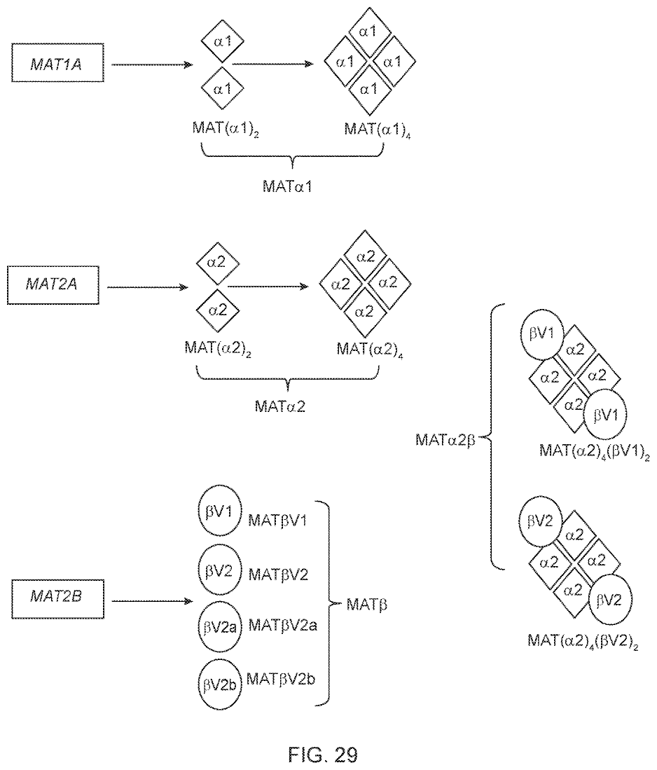

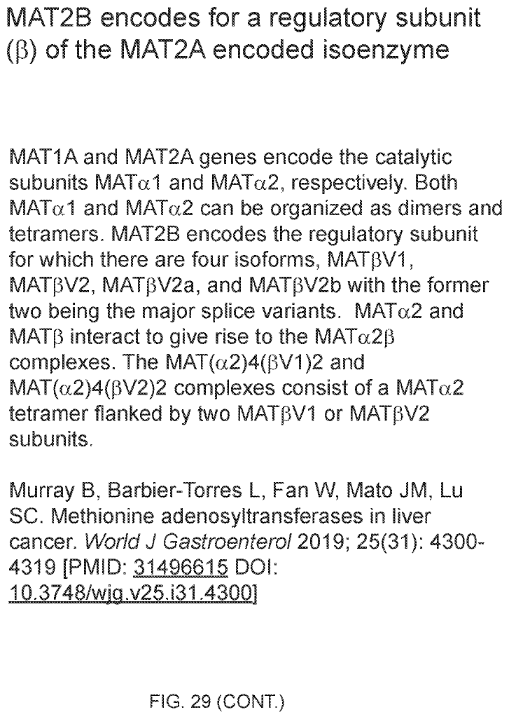

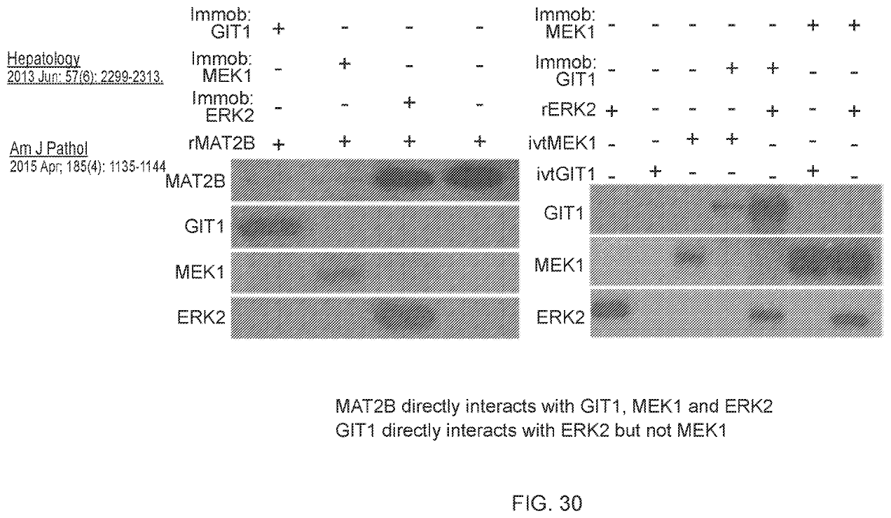

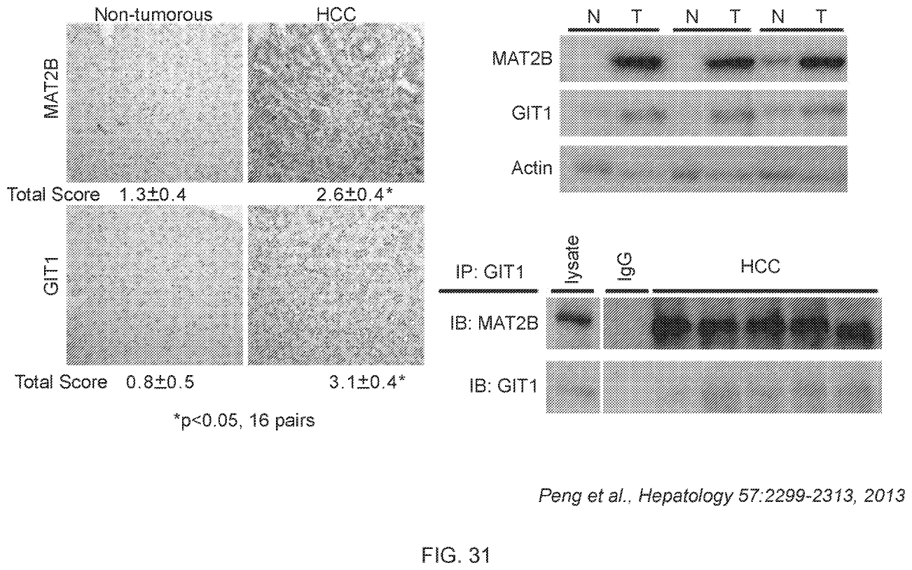

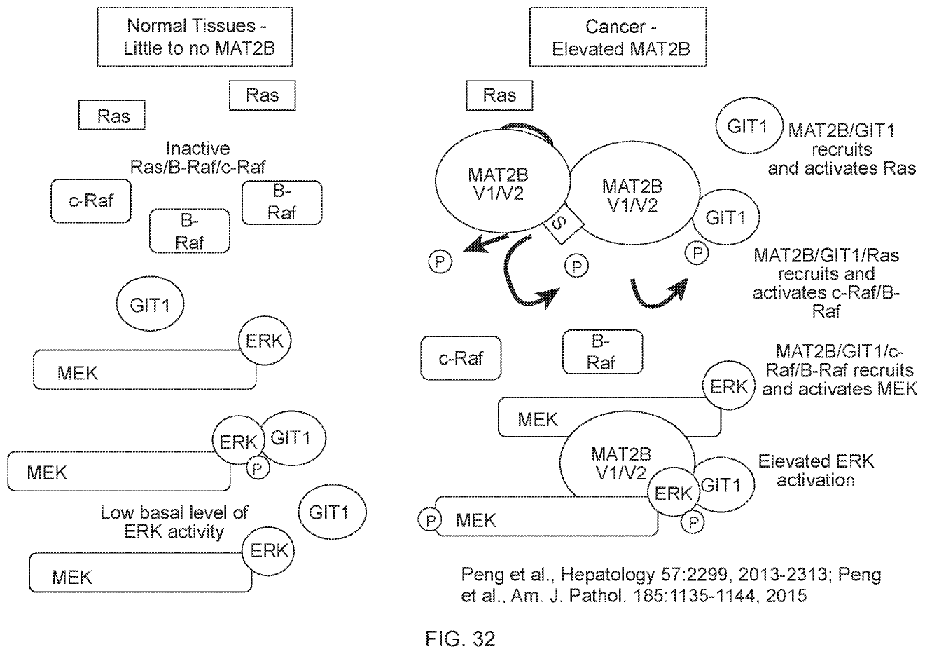

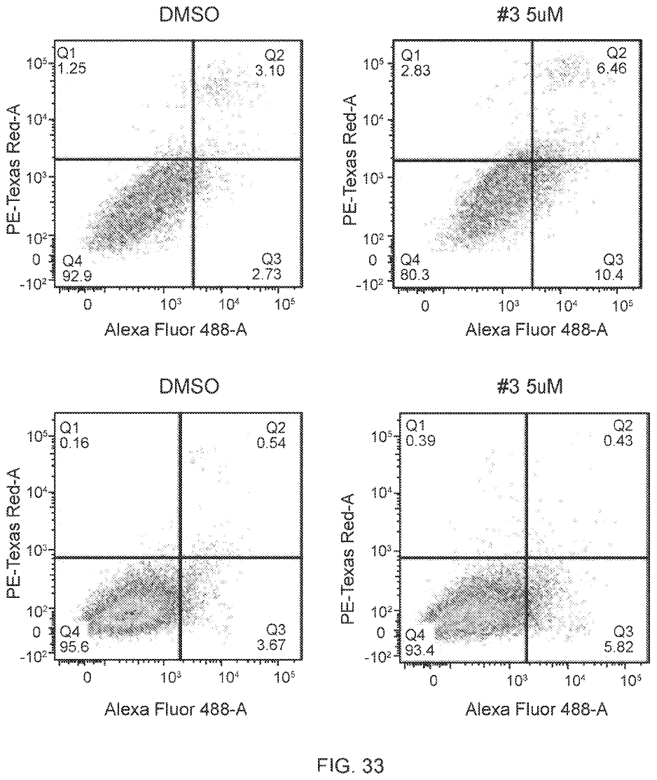

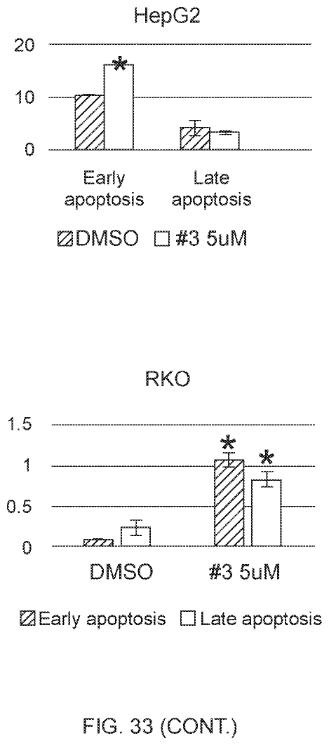

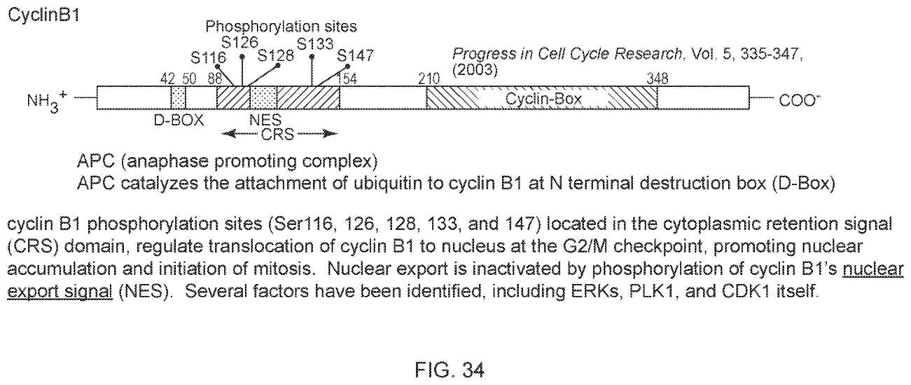

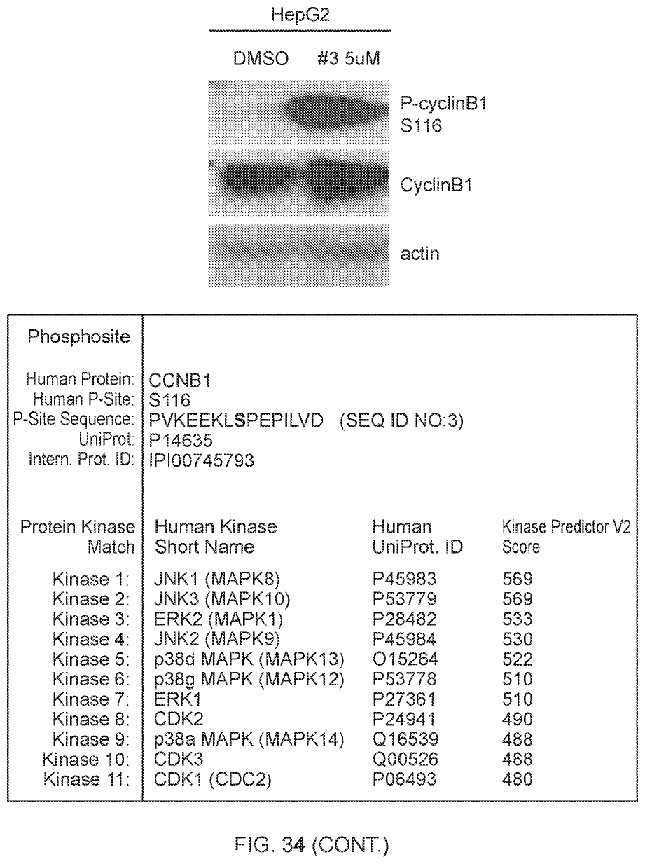

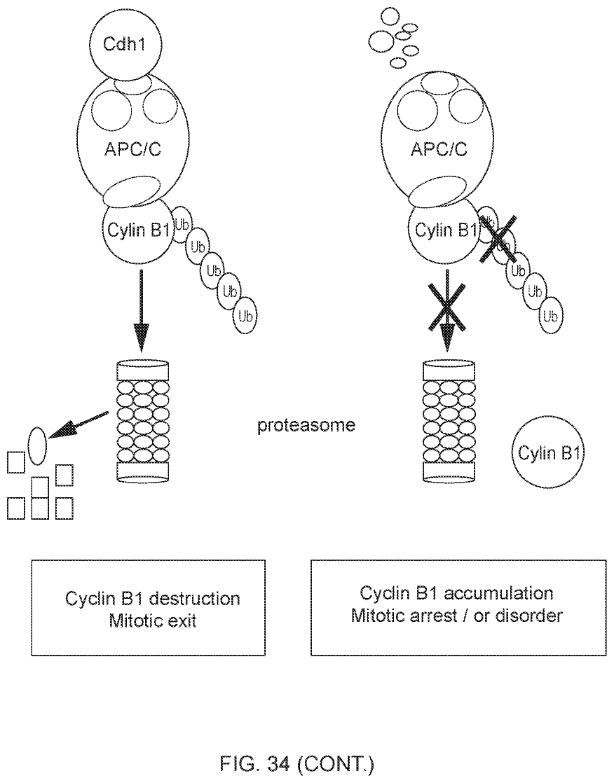

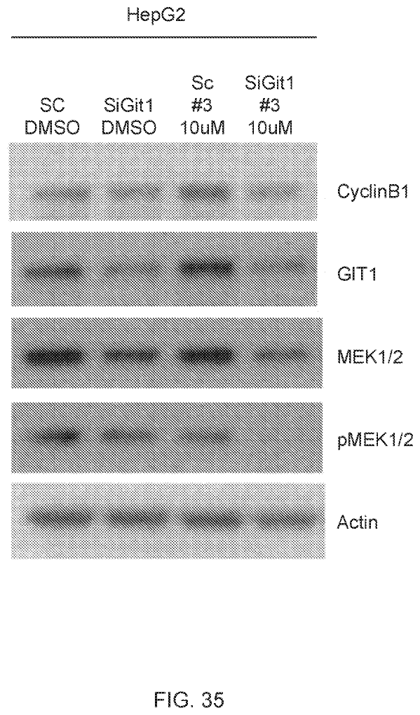

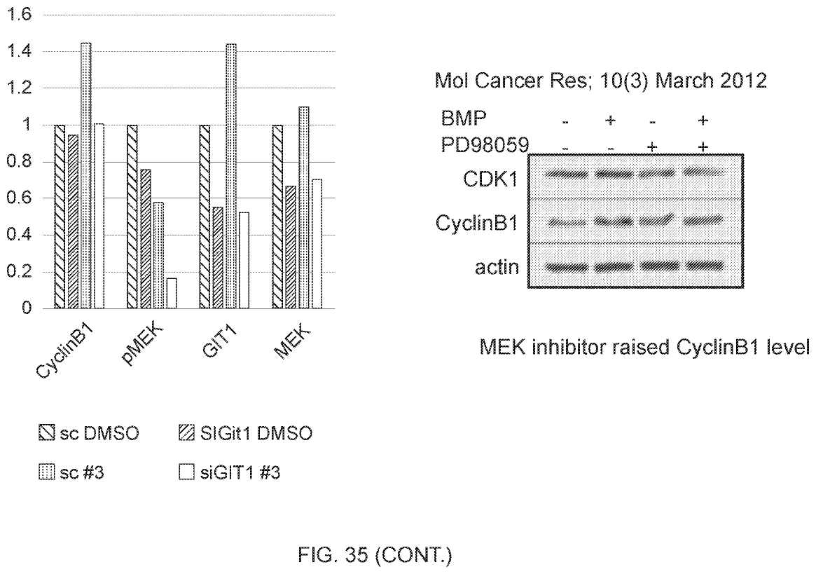

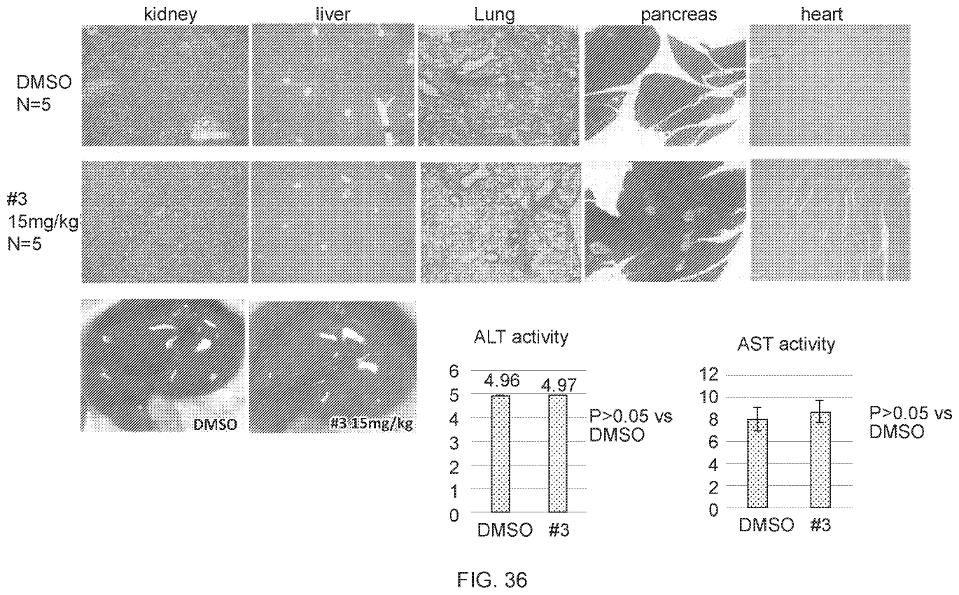

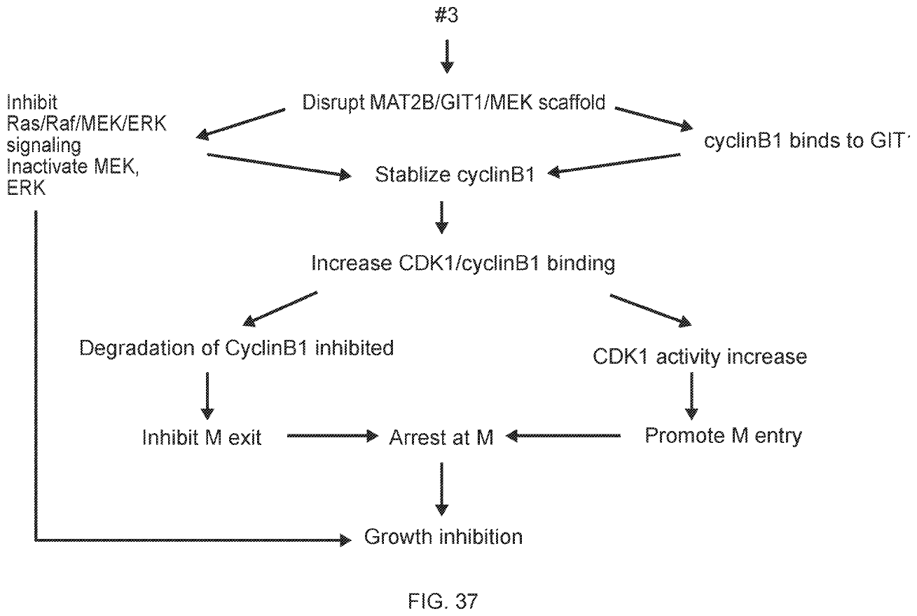

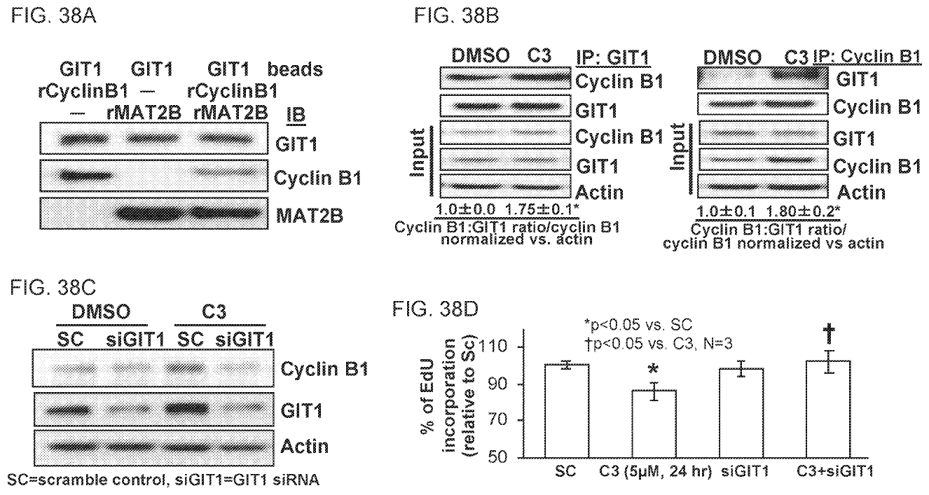

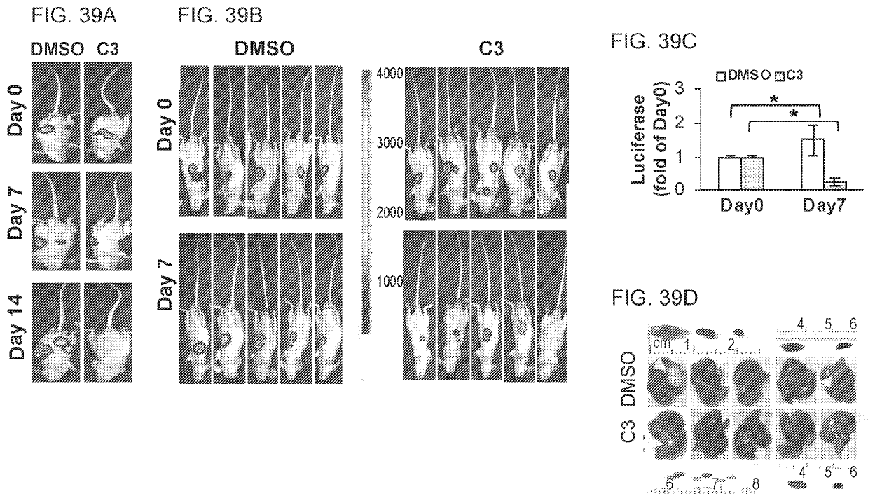

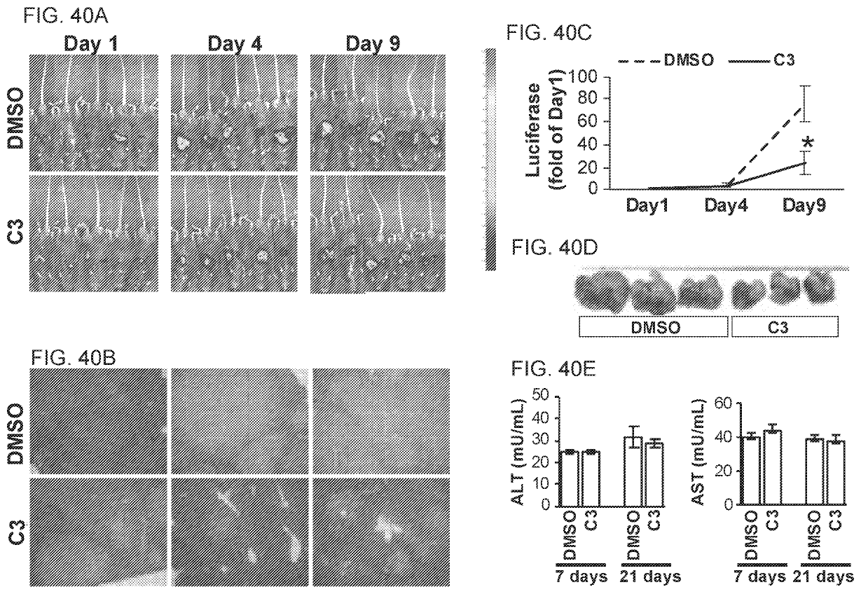

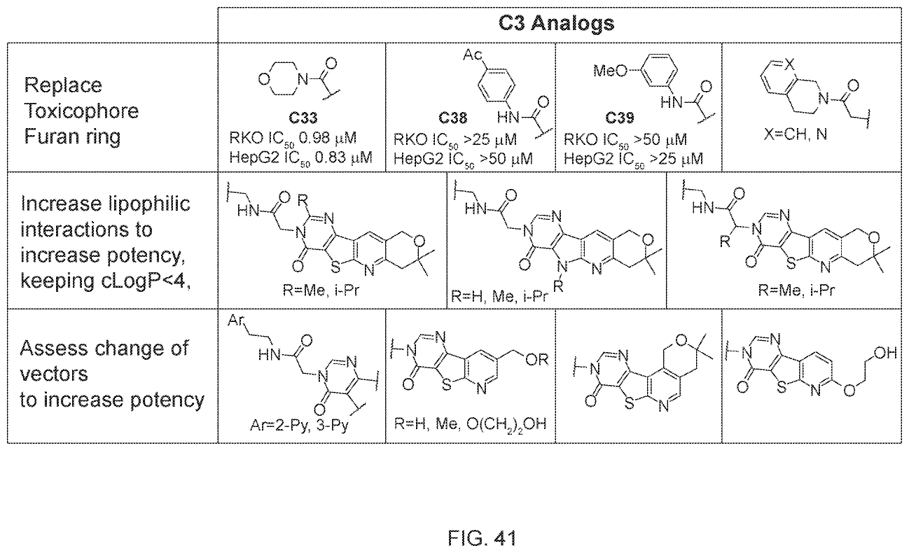

GIT1 WT was mutated at proline 730, 731, 733, and 736 residues to alanine (PPEPGAPVD (SEQ ID NO:1)→AABAGAAVD (SEQ ID NO:2)) (GIT1mut) and overexpressed in RKO cells for 48 hours, followed by co-IP with anti-cyclin BI antibody and blotting for cyclin B1 and GIT1. FIG. 25 shows results of co-immunoprecipitation with cyclin B1, with densitometric values from n=3 (*p<0.05 vs. GIT1 wt). Less interaction between GIT1 mutant and cyclin Bl supports that GIT1 aa730-738 is the region that interacts with cyclin B1.

### Example 5

### Synthesis of Compound C33

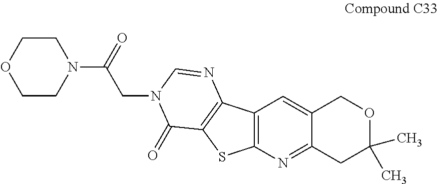 [SMILES: CC1(C)Cc2nc3sc4c(=O)n(CC(=O)N5CCOCC5)cnc4c3cc2CO1]

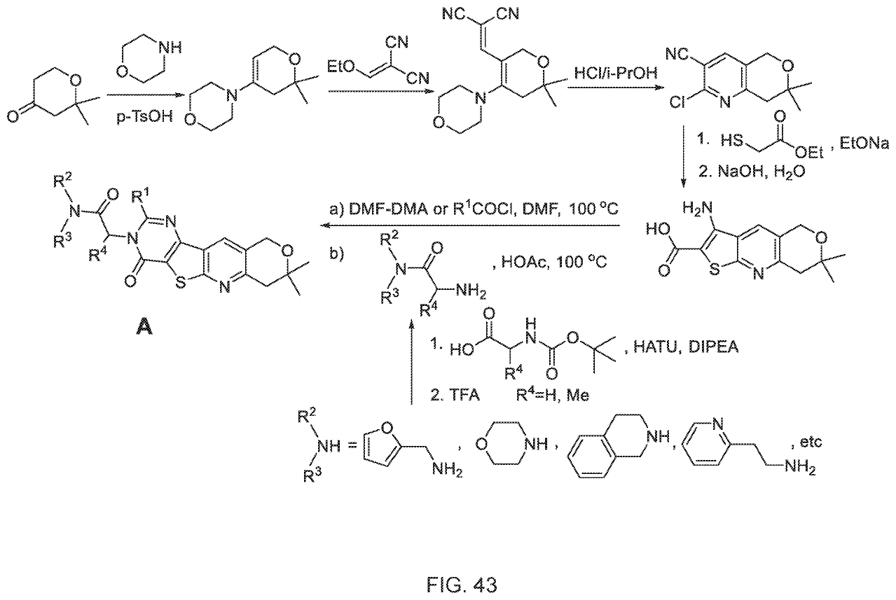

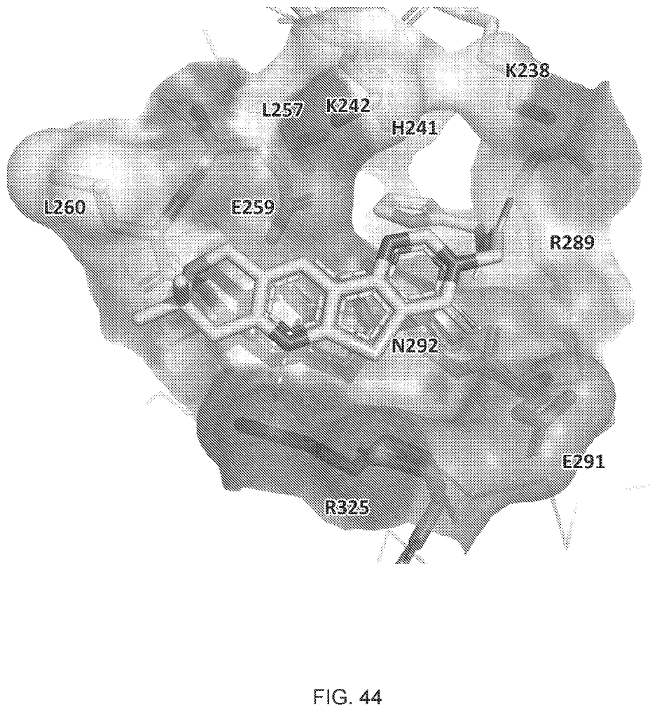

Compound C33 was prepared according to the general synthetic approach shown in FIG. 43. RKO IC₅₀ 0.98 μM. HepG2 IC₅₀ 0.83 μM.

### Example 6

### Synthesis of Compound C38

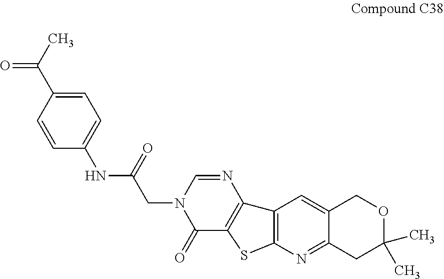 [SMILES: CC(=O)c1ccc(NC(=O)Cn2cnc3c(sc4nc5c(cc43)COC(C)(C)C5)c2=O)cc1]

Compound C38 was prepared according to the general synthetic approach shown in FIG. 43. RKO IC₅₀>25 uM. HepG2 IC₅₀>50 uM.

### Example 7

### Synthesis of Compound C39

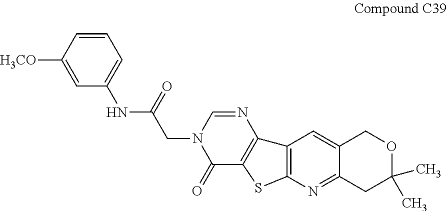 [SMILES: COc1cccc(NC(=O)Cn2cnc3c(sc4nc5c(cc43)COC(C)(C)C5)c2=O)c1]

Compound C39 was prepared according to the general synthetic approach shown in FIG. 43. RKO IC₅₀>50 uM. HepG2 IC₅₀>25 uM.

### Example 8

### Effects of Compound C33 on MTT in RKO and HepG2 Cells

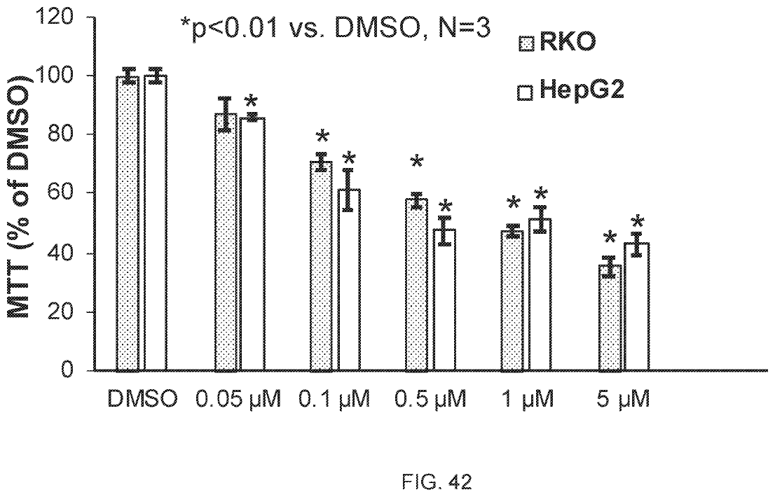

MTT assay showed Compound C33 has (sub-micromolar) potency against RKO and HepG2 (IC₅₀ 0.98 and 0.83 μM, respectively (FIG. 42). Compound C33 has improved (sub-macromolar) potency against RKO and HepG2 over that of Compound C3 (RKO/HepG2 IC₅₀ ca. 20 uM) with no toxicity in human or mouse hepatocytes (data not shown).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

1. A compound of Formula (A): 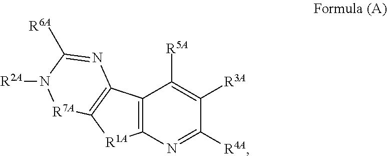
wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{3A} is H, halo, optionally substituted alkyl, or OR^{9A}, where R^{9A} is H or optionally substituted alkyl;
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl; and
R^{7A} is C═O or CH₂; or
R^{3A} and R^{4A} can be linked together to form a ring, wherein the ring is optionally substituted; or
R^{3A} and R^{5A} can be linked together to form a ring, wherein the ring is optionally substituted, with the proviso that the compound of Formula (A) is not

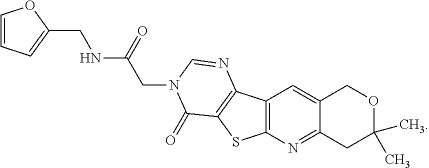 [SMILES: CC1(C)Cc2nc3sc4c(=O)n(CC(=O)NCc5ccco5)cnc4c3cc2CO1]

2. The compound of claim 1, wherein R^{2A} is: 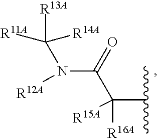
wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl; or
wherein R^{2A} is: 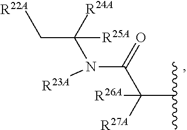
wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl; or
wherein R^{2A} is: 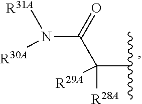
wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted; or
wherein R^{2A} is: 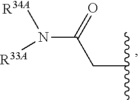
wherein,
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

3.-17. (canceled)

18. The compound of claim 1, having a structure of Formula (A-1): 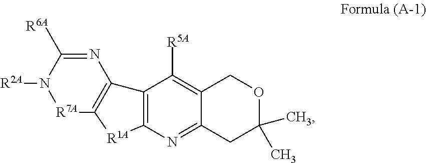
wherein:
R^{1A} is S, O, CH₂, C(CH₃)₂, or NR^{8A} where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{5A} is H, halo, or optionally substituted alkyl;
R^{6A} is H, halo, or optionally substituted alkyl; and
R^{7A} is C═O or CH₂.

19. The compound of claim 18, wherein R^{2A} is: 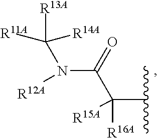
wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl; or
wherein R^{2A} is: 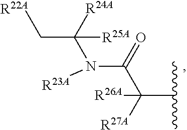
wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl:
R^{25A} is H, halo, or optionally substituted alkyl:
R^{26A} is H, halo, or optionally substituted alkyl; and
R^{27A} is H, halo, or optionally substituted alkyl; or
wherein R^{2A} is: 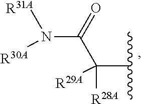
wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted; or
wherein R^{2A} is: 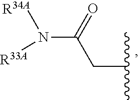
wherein,
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

20.-34. (canceled)

35. The compound of claim 1, having a structure of Formula (A-2): 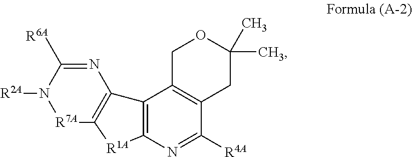
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{4A} is H, halo, optionally substituted alkyl, or OR^{10A}, where R^{10A} is H or optionally substituted alkyl;
R^{6A} is H, halo, or optionally substituted alkyl; and
R^{7A} is C═O or CH₂.

36. The compound of claim 35, wherein R^{2A} is: 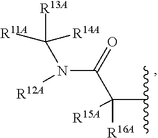
wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{6A} is H, halo, or optionally substituted alkyl; or 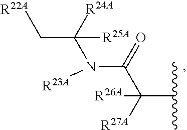
wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl; or
wherein R^{2A} is: 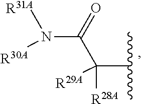
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted; or 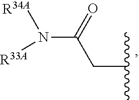
wherein:
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

37.-51. (canceled)

52. The compound of claim 1, having a structure of Formula (A-3): 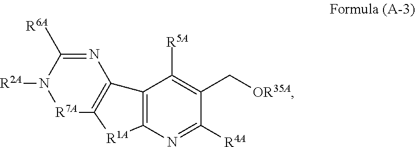
wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl;
R^{7A} is C═O or CH₂; and
R^{35A} is H, CH₃, or CH₂CH₂OH.

53. The compound of claim 52, wherein R^{2A} is: 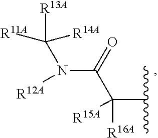
wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl; or
wherein R^{2A} is: 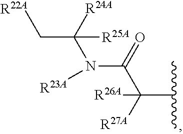
wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl; or
wherein R^{2A} is: 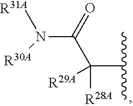
wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted; or
wherein R^{2A} is: 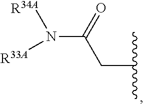
wherein,
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

54.-68. (canceled)

69. The compound of claim 1, having a structure of Formula (A-4): 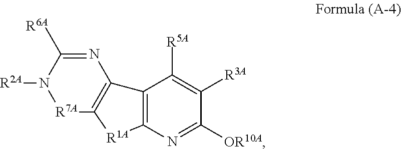
wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{3A} is H, halo, optionally substituted alkyl, or OR^{9A}, where R^{9A} is H or optionally substituted alkyl;
R^{5A} is H, halo, or optionally substituted alkyl;
R^{6A} is H, halo, or optionally substituted alkyl;
R^{7A} is C═O or CH₂; and
R^{10A} is H, CH₃, or CH₂CH₂OH.

70. The compound of claim 69, wherein R^{2A} is: 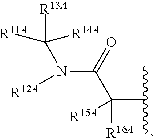
wherein:
R^{11A} is H, halo, optionally substituted alkyl, optionally substituted aryl, optionally substituted heteroaryl, optionally substituted cyclyl, optionally substituted heterocyclyl;
R^{12A} is H or optionally substituted alkyl;
R^{13A} is H, halo, or optionally substituted alkyl;
R^{14A} is H, halo, or optionally substituted alkyl;
R^{15A} is H, halo, or optionally substituted alkyl; and
R^{16A} is H, halo, or optionally substituted alkyl; or
wherein R^{2A} is: 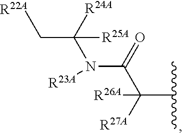
wherein:
R^{22A} is halo, optionally substituted alkyl, optionally substituted aryl, optionally substituted heteroaryl, optionally substituted cyclyl, or optionally substituted heterocyclyl;
R^{23A} is H or optionally substituted alkyl;
R^{24A} is H, halo, or optionally substituted alkyl;
R^{25A} is H, halo, or optionally substituted alkyl;
R^{26A} is H, halo, or optionally substituted alkyl; and
R^{27A} is H, halo, or optionally substituted alkyl; or
wherein R^{2A} is: 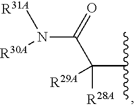
wherein:
R^{28A} is H, halo, or optionally substituted alkyl;
R^{29A} is H, halo, or optionally substituted alkyl;
R^{30A} is H or optionally substituted alkyl; and
R^{31A} is H or optionally substituted alkyl; or
R^{30A} and R^{31A} can be linked together to form a ring, wherein the ring is optionally substituted; or
wherein R^{2A} is: 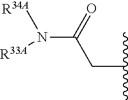
R^{33A} is H, optionally substituted alkyl; and
R^{34A} is H, optionally substituted alkyl, optionally substituted aryl, optionally substituted heteroaryl, optionally substituted cyclyl, or optionally substituted heterocyclyl; or
R^{33A} and R^{34A} can be linked together to form a ring, wherein the ring is optionally substituted.

71.-85. (canceled)

86. The compound of claim 1, selected from the group consisting of:

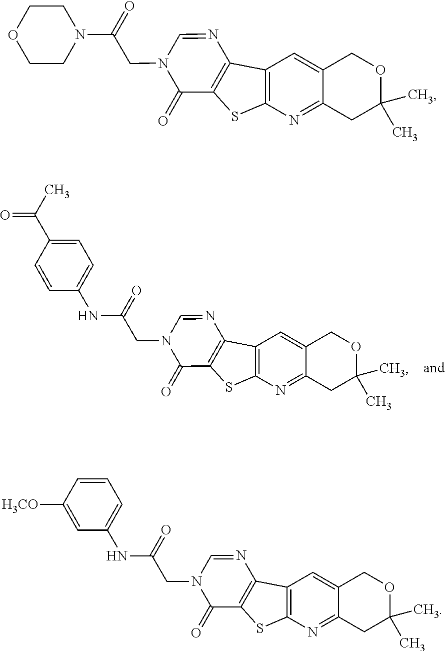 [SMILES: CC1(C)Cc2nc3sc4c(=O)n(CC(=O)N5CCOCC5)cnc4c3cc2CO1 ; CC(=O)c1ccc(NC(=O)Cn2cnc3c(sc4nc5c(cc43)COC(C)(C)C5)c2=O)cc1 ; COc1cccc(NC(=O)Cn2cnc3c(sc4nc5c(cc43)COC(C)(C)C5)c2=O)c1]

87. The compound of claim 1, wherein the compound is:

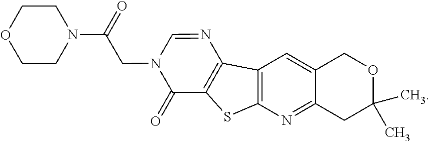 [SMILES: CC1(C)Cc2nc3sc4c(=O)n(CC(=O)N5CCOCC5)cnc4c3cc2CO1]

88. The compound of claim 1, wherein the compound is:

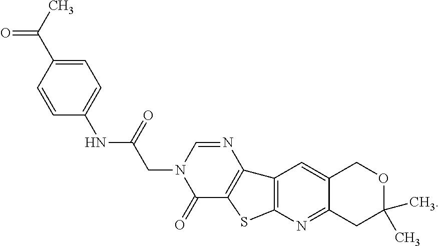 [SMILES: CC(=O)c1ccc(NC(=O)Cn2cnc3c(sc4nc5c(cc43)COC(C)(C)C5)c2=O)cc1]

89. The compound of claim 1, wherein the compound is:

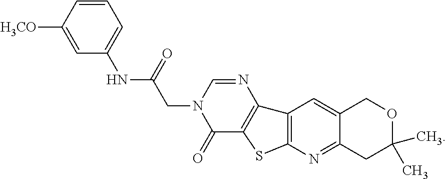 [SMILES: COc1cccc(NC(=O)Cn2cnc3c(sc4nc5c(cc43)COC(C)(C)C5)c2=O)c1]

90. The compound of claim 1, wherein the compound is a pharmaceutically acceptable salt, solvate, hydrate, polymorph, co-crystal, tautomer, stereoisomer, or isotopically labeled derivative thereof.

91. A pharmaceutical composition comprising an inhibitor of GIT1's normal interactome.

92. The pharmaceutical composition of claim 91, wherein the inhibitor of GIT1 is a compound of Formula (A): 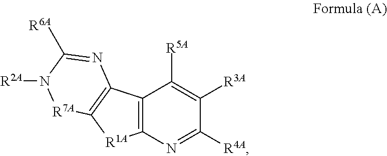
wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{3A} is H, halo, optionally substituted alkyl, or OR^{9A}, where R^{9A} is H or optionally substituted alkyl;
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl; and
R^{7A} is C═O or CH₂; or
R^{3A} and R^{4A} can be linked together to form a ring, wherein the ring is optionally substituted; or
R^{3A} and R^{5A} can be linked together to form a ring, wherein the ring is optionally substituted, with the proviso that the compound of Formula (A) is not

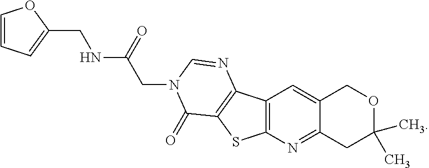 [SMILES: CC1(C)Cc2nc3sc4c(=O)n(CC(=O)NCc5ccco5)cnc4c3cc2CO1]

93.-99. (canceled)

100. The pharmaceutical composition of claim 91, wherein the inhibitor of GIT1 is:

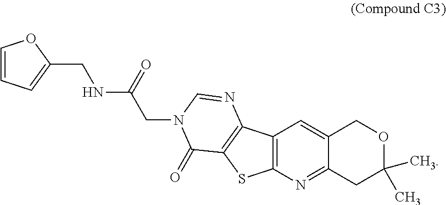 [SMILES: CC1(C)Cc2nc3sc4c(=O)n(CC(=O)NCc5ccco5)cnc4c3cc2CO1]

101. A method of treating, preventing, reducing the likelihood of having, reducing the severity of and/or slowing the progression of a condition in a subject comprising:
administering a therapeutically effective amount of an inhibitor of GIT1's normal interactome to the subject, thereby treating, preventing, reducing the likelihood of having, reducing the severity of and/or slowing the progression of the condition in the subject,
wherein the condition is cancer or a condition associated with an increased expression of GIT1 and MAT2B.

102.-104. (canceled)

105. The method of claim 101, wherein the inhibitor of GIT1 is a compound of Formula (A): 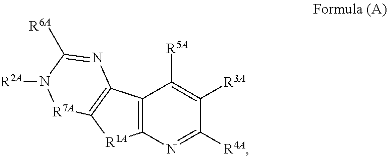
wherein:
R^{1A} is S, O, CH₂, C(CH₃)₂, or NR^{8A}, where R^{8A} is H or optionally substituted alkyl;
R^{2A} is H, optionally substituted alkyl, optionally substituted carbonyl, optionally substituted aryl, optionally substituted heteroaryl, optionally substituted cyclyl, or optionally substituted heterocyclyl;
R^{3A} is H, halo, optionally substituted alkyl, or OR^{9A}, where R^{9A} is H or optionally substituted alkyl;
R^{4A} is H, halo, optionally substituted alkyl, or OR^{10A}, where R^{10A} is H or optionally substituted alkyl;
R^{5A} is H, halo, or optionally substituted alkyl;
R^{6A} is H, halo, or optionally substituted alkyl; and
R^{7A} is C═O or CH₂; or
R^{3A} and R^{4A} can be linked together to form a ring, wherein the ring is optionally substituted; or
R^{3A} and R^{5A} can be linked together to form a ring, wherein the ring is optionally substituted, with the proviso that the compound of Formula (A) is not

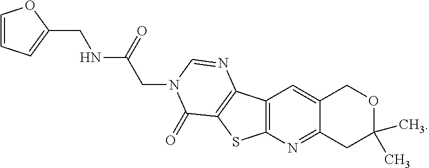 [SMILES: CC1(C)Cc2nc3sc4c(=O)n(CC(=O)NCc5ccco5)cnc4c3cc2CO1]

106.-113. (canceled)

114. The method of claim 101, wherein the inhibitor of GIT1 is:

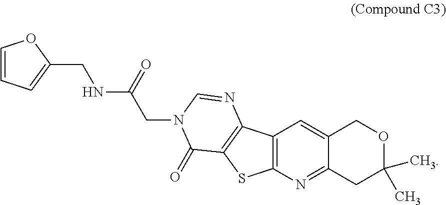 [SMILES: CC1(C)Cc2nc3sc4c(=O)n(CC(=O)NCc5ccco5)cnc4c3cc2CO1]

115.-118. (canceled)


SEQUENCE LISTING (3 sequences)

SEQ ID NO: 1 - protein, 9 aa, Homo sapiens
PPEPGAPVD

SEQ ID NO: 2 - protein, 9 aa, Homo sapiens
AAEAGAAVD

SEQ ID NO: 3 - protein, 15 aa, Homo sapiens
PVKEEKLSPEPILVD